(19) United States
(12) Patent Application Publication
(10) Pub. No.: US 2024/0003059 A1
(43) Pub. Date: Jan. 4, 2024

(54) SOLUTION ELECTROWRITING

(71) Applicant: CORNELL UNIVERSITY, Ithaca, NY (US)

(72) Inventors: Anthony D'Amato, Ithaca, NY (US); Yadong Wang, Ithaca, NY (US); Yen-Lin Wu, Ithaca, NY (US); Yu-An Chien, Ithaca, NY (US)

(21) Appl. No.: 18/249,938

(22) PCT Filed: Oct. 22, 2021
(86) PCT No.: PCT/US2021/056351

§ 371 Date: Apr. 20, 2023

Related U.S. Application Data
(60) Provisional application No. 63/104,493, filed on Oct. 22, 2020.

(51) Int. Cl. D01D 5/00; D01D 1/02; D04H 1/728; D04H 3/04; D04H 3/011; D04H 3/147; A61L 27/54; A61L 27/50; A61L 27/26

(52) U.S. Cl. CPC D01D 5/0069; D01D 5/0038; D01D 1/02; D04H 1/728; D04H 3/04; D04H 3/011; D04H 3/147; D01D 5/0076; A61L 27/54; A61L 27/507; A61L 27/26; D10B 2509/06; D10B 2331/041; D10B 2331/04; D10B 2401/12

(57) ABSTRACT

Solution electro writing systems, solution electrowriting methods, products made by the solution electrowriting systems or methods, and uses thereof. A solution electro written product can include one or more layer(s) of fibers in a predetermined pattern with various degrees of fiber fusion, fiber stacking, fiber porosity, or any combination thereof. A solution electro written product can be tubular or flat. A solution electro written product can be a conduit, a web, a patch, a cuff, or a shape of at least a portion of an organ, or the like. A solution electro written product can comprise polymer(s), such as, for example, biocompatible and/or biodegradable polymer(s). A solution electro written product can be used for tissue grafts, including arterial grafts, such as, for example, arteriovenous grafts.

### CROSS-REFERENCE TO RELATED APPLICATIONS

This application claims the benefit of U.S. Provisional Patent Application No. 63/104,493, filed Oct. 22, 2020, the contents of the above-identified application are hereby fully incorporated herein by reference in their entirety.

### BACKGROUND OF THE DISCLOSURE

Electrospinning is widely used to make fibrous constructs in biomedical research because it creates fibrous materials with fibers on the nano- to micro-scale, the scales many cells find themselves in. Industrial usage of electrospinning is mostly limited to filtration because the conventional processing method doesn't allow fine control of the structural features of the final product. Therefore, the control of current industrial product properties is largely limited to fiber diameter and statistical average pore size.

Electrospinning is one of the most common methods to produce fibrous material constructs with fibers on the nano- to micro-scale. A number of technologies have been used to improve control during electrospinning. Near-field electrospinning combined with a rotating mandrel has been performed using a technique known as melt electrospinning (referred to as near-field melt electrospinning writing). 3D printing is another technology that uses a similar approach to address this problem, albeit with its own set of limitations.

Electrospinning has gained increasing popularity in recent decades as an additive manufacturing technique used to produce fibrous scaffolds with nano- to micrometer fiber diameters. While electrospinning has the benefits of low cost and ease of use, the process itself lacks control and precision. This is due to the whipping instability necessary for fiber jets emanating from the spinneret to stretch to small diameters. As a result, other material fabrication methods have become more favorable options when precision control of scaffold patterning, resolution, or morphology is required. Recent developments with the technique “near-field electrospinning” focus on negating the inherent instability of conventional electrospinning. This is achieved by significantly decreasing the gap distance between the spinneret and fiber collector. This enables electrospinning at drastically lower applied voltages, and solution flow rates (V) to achieve similar fiber diameters without causing whipping instability.

Melt electrowriting has been used to fabricate tubular scaffolds. Tubular scaffolds have seen wide usage in many applications including endotracheal tubes, vascular grafts, and nerve growth conduits, to name a few. Melt electrowriting is theoretically easier to control than solution electrowriting due to the non-conductive nature of polymer melts. Arguably the largest disadvantages of melt vs. solution are the limited selection of polymers with melting points that are reasonably low, to achieve acceptable viscosity for extrusion, and the limited selection of additives which are stable at the increased temperature needed to melt the polymers. Therefore, the equipment is still specialized. Mitigation of this limitation by melt electrowriting a chemically cross-linked hydrogel have been attempted.

A desirable application for vascular grafts is hemodialysis. Dialysis is the only treatment option for over 97% of the end-stage renal disease (ESRD) patients who cannot get a transplant. Approximately 90% of these patients are on hemodialysis. Hemodialysis requires a high flow filtration system. Therefore, a hemocompatible material is desirable. When an implanted self-contained artificial kidney becomes a reality, then a relatively low flow system will be feasible. However, it will place even higher requirement on hemocompatibility and overall biocompatibility. Until such a device is available, addressing critical deficiencies of hemodialysis will impact the care of ESRD patients.

Vascular access for hemodialysis, using either an arteriovenous (AV) fistula or AV graft, is often regarded as the Achilles's heel because both access strategies have inherent deficiencies. The AV fistula is the gold standard dialysis access and national guidelines on “fistula first” maximizes the number of patients who can dialyze through AV fistulae. However, approximately 39% of AV fistula fails to mature. For matured AV fistula, −40% need interventions to maintain sufficient intra-access blood flow and 29% are abandoned within a year. A fistula is an “abnormal connection between vessels or organs”. In the context of dialysis, connecting an artery directly to a vein induces a huge spike in pressure and turbulent flow, deforming the outflow vein and inflaming its cells. This mechanical overload drives the vein to thicken and adopt arterial like properties to satisfy the newly founded over-pressurized environment. This maturation process is very successful in the setting of a healthy vein. The greatest limitation in AV fistula creation is the availability of suitable veins. Because patients with ESRD often have numerous comorbidities and are elderly, the arm veins of these patients are often scarred from numerous prior punctures for blood draws and intravenous catheters. While AV fistulae are superior dialysis accesses, a large fraction of the ESRD population suffer from poor venous anatomy. The placement of AV fistula in the setting of poor veins results in multiple reinterventions and abandoned accesses and this creates a huge financial and emotional burden on patients and increases costs for the healthcare system.

In the setting of poor veins, AV grafts are placed. The primary patency rate of grafts are 66% at one year, dropping to 40%, 27% and 18% after two, three and five years respectively. Current grafts are susceptible to stenosis, neointimal hyperplasia, rejection, and infection. AV grafts have two source materials: synthetic polymer and blood vessels derived from human or bovine sources. They face even more challenges than the fistula. Most synthetic grafts used for AV access creation are made with expanded polytetrafluoroethylene (ePTFE). The elastic modulus of PTFE is approximately 500 MPa depending on preparation. The moduli of veins and arteries vary depending on pressure, but the general range is approximately 15 kPa to 3 MPa, 33,000 to 160 times softer than PTFE. The difference in stiffness between graft and vein is much larger than that between artery and vein. Moreover, synthetic AV grafts are thick and tough to handle repeated cannulation. On the other hand, vein is thin and soft. The difference in thickness compounds the already stiff graft and further increases the compliance mismatch. Endothelial coverage on PTFE is very limited. The three-fold challenge of huge compliance mismatch, thrombogenicity and turbulent flow leads to graft failure. In grafts derived from preserved human vessels or fixed decellularized animal vessels, the mismatch of mechanical properties of artery and vein persists: the graft can approximate one compliance or the other, but not both. Further limitations of allografts include poor long-term patency and allosensitization that can impact the patient's ability to receive a renal transplant. This same immune response can also result in graft inflammation and scarring over time. The xenografts are decellularized and do not have the same immune sensitization but have been shown to have poor primary patency (30% at 1 year), worse than that seen with PTFE (43% at 1 year), and similar infection rates. It only outperforms PTFE in secondary patency.

When AV fistulae mature, their long-term patency is desirable. However, up to 25-40% never mature due to poor venous anatomy or poor arterial flows and a significant number of patients are not candidates due to inadequate vein conduit. AV grafts have a lower immediate failure rate but long-term patency is inferior and require multiple interventions to maintain patency due predominantly to the thrombogenicity of the graft material and the compliance mismatch at the venous anastomoses.

The human body has inherent healing capabilities. Harvesting this capability may lead to the transformation of degradable grafts into autologous vascular conduits, as shown herein in preclinical study in animals. Vascular grafts started with Dacron® in the late 1950s. Studies on classic vascular grafts (Dacron® and PTFE) suggest that patients would remodel synthetic grafts with their own cells. The key is what types of host cells are recruited and how they remodel the grafts. Most of the host cells in remodeled Dacron and PTFE grafts are fibroblasts. Host remodeling is largely fibrotic with small amount of host tissue in the interstices of the Dacron graft and occasional partial endothelialization of the lumen. Therefore, existing synthetic vascular grafts are challenged by limited cell infiltration and fibrosis. This pathological remodeling does not improve its mechanical properties and contribute to thrombogenicity. The human body is in a dynamic steady state with constant production and degradation of molecules. Even the remarkably stable extracellular protein, elastin, can be degraded by matrix metalloproteases. The only non-degradable products that the human body makes are outgrowing structures like hairs, fingernails, and toenails. When growing inwards, these non-degradable structure causes inflammation. Thus, philosophically, it is believed these non-degradable grafts result in a dysfunctional healing process rather than harnessing it.

The premise of tissue engineering and regenerative medicine is to harness the innate healing potential of the body. Vascular grafts have evolved from cell-laden constructs to decellularized tissue engineered grafts. These decellularized grafts undergo significant host remodeling after implantation leading to a useful conduit. The host remodeling of these tissue-engineered conduits leads to endothelialization and host cell infiltration in the interstices of the graft. This disclosure reveals three facts: 1. The innate power of the human body to heal itself is significant even in patients with underlying systemic diseases; 2. Host cells do migrate into the interstices of a graft; and 3. Cell seeding benefits the grafts by depositing extracellular matrix, mostly collagen, in the production stage. However, the presence of cells in the graft is unnecessary for positive host remodeling. Cell sourcing, seeding, and culturing prolong and complicate graft fabrication, render it difficult to store and transport, and drastically increase the cost. Furthermore, the dense matrix of the decellularized graft hinders host cell infiltration, which takes 18 weeks to become significant.

### SUMMARY OF THE DISCLOSURE

In an aspect, the present disclosure provides methods of making fibrous products. The methods can be carried out using solution electrowriting systems. In various examples, a method comprises providing a solution electrowriting system comprising: one or more nozzle(s); a material supply system comprising one or more reservoir(s) fluidically coupled to the nozzle(s) and configured to supply one or more fluid stock(s) to the nozzle(s) thereby ejecting one or more jet stream(s) of the fluid stock(s) from the nozzle(s); a collector system configured to collect one or more fiber(s) formed by the jet stream(s) ejected from the nozzle(s); and one or more power source(s) configured to provide one or more electric potential(s) to each of the nozzle(s) and, optionally, to the collector system, thereby providing one or more electric potential difference(s) between the collector system and each of the nozzle(s). In various examples, the method further comprises ejecting the fluid stream(s) of the fluid stock(s) from the nozzle(s) to form the fiber(s). In various examples, the method further comprises collecting the fiber(s) with the collector system to form a fibrous product comprising one or more fiber(s) arranged in a predetermined pattern. In various examples, the method further comprises releasing the fibrous product from collector system, where a desired fiber fusion and/or a desired fiber stacking is observed in the fibrous product. The method can further comprise, after the collecting and/or the releasing, heating and/or drying the fibrous product.

A method can use a system comprising various fluid stock(s). In various examples, each fluid stock comprises a solution comprising at least one first solvent and, optionally, at least one second solvent, and one or more material(s) configured to form at least a portion of a fiber upon ejection of the jet stream(s) of the fluid stock(s) from the nozzle(s). In various examples, the material(s) is/are dissolvable in at least one of the solvent(s) to form a solution.

A method can achieve a desired level of fiber fusion using various fluid stock(s) comprising at least one first solvent and at least one second solvent having various boiling point(s). In various examples, the fluid stock(s) comprise at least one first solvent having a boiling point of less than about 80° C., and at least one second solvent having a boiling point of at least about 80° C. or greater. In various examples, the fluid stock(s) comprise(s) at least one first solvent and at least one second solvent, where the boiling point of the at least one second solvent is from about 10° C. to about 200° C., including all 0.1° C. values and ranges therebetween, higher than the boiling point of the at least one first solvent. In various examples, the at least one first solvent is chosen from diethyl ether, dichloromethane (DCM), acetone, 1,1,1,3,3,3-hexafluoroisopropanol (HFIP), chloroform, methanol, tetrahydrofuran (THF), trifluoroethanol (TFE), ethanol, acetonitrile, cyclohexane, benzene, ethyl acetate, hexane, trifluoroacetic acid, isopropanol, and the like, and any combination thereof. In various examples, the at least one second solvent is chosen from water, dioxane, toluene, pyridine, N,N-dimethylformamide (DMF), anisole, dimethyl sulfoxide (DMSO), 1,2-dichloroethane, triethylamine, heptane, butanol, acetic acid, xylene, diglyme (diethylene glycol diethyl ether), and the like, and any combination thereof. In various examples, the volume ratio of the at least one first solvent to the at least one second solvent is from about 1:99 to about 99:1, including all integer volume ratio values and ranges therebetween. In various examples, the fibrous product comprises a plurality of fusion points between respective portions of at least two adjacent intersected fibers, such that for each fusion point, a bottom surface of a first fiber is bonded to a top surface of a second fiber. In various examples, the plurality of fusion points between adjacent intersected fibers is observed at an average frequency of from about 5% to about 99%, including all 0.1% values and ranges therebetween. In various examples, the fibers comprise a plurality of fusion points between two intersected fibers, such that for each fusion point, a bottom surface of a first fiber is bonded to a top surface of a second fiber. In various examples, the plurality of fusion points between two intersected fibers is observed at an average frequency of from about 5% to about 99%, including all 0.1% values and ranges therebetween.

A method can achieve a desired level of fiber fusion using various fluid stock(s) comprising at least one first solvent having various boiling point(s). In various examples, the fluid stock(s) comprise(s) at least one first solvent having a boiling point of from about 70° C. to about 120° C., including all 0.1° C. values and ranges therebetween. In various examples, the at least one first solvent is chosen from trifluoroethanol (TFE), ethanol, acetonitrile, cyclohexane, benzene, ethyl acetate, hexane, trifluoroacetic acid, isopropanol, water, dioxane, toluene, pyridine, and the like, and any combination thereof. In various examples, the fibrous product comprises a plurality of fusion points between respective portions of at least two adjacent intersected fibers, such that for each fusion point, a bottom surface of a first fiber is bonded to a top surface of a second fiber. In various examples, the plurality of fusion points between adjacent intersected fibers is observed at an average frequency of from about 5% to about 99%, including all 0.1% values and ranges therebetween. In various examples, the fibers comprise a plurality of fusion points between two intersected fibers, such that for each fusion point, a bottom surface of a first fiber is bonded to a top surface of a second fiber. In various examples, the plurality of fusion points between two intersected fibers is observed at an average frequency of from about 5% to about 99%, including all 0.1% values and ranges therebetween.

A method can achieve a desired level of fiber stacking using various fluid stock(s) comprising at least one first solvent and at least one second solvent having various dipole moment(s). In various examples, the fluid stock(s) comprise(s) at least one first solvent and at least one second solvent, where the at least one first solvent has a dipole moment of from about 1.5 D to about 4.2 D, including all 0.1 D values and ranges therebetween, and the at least one second solvent has a dipole moment of from about 0 D to less than about 1.5 D, including all 0.1 D values and ranges therebetween. In various examples, the fluid stock(s) comprise(s) at least one first solvent and at least one second solvent, and where the dipole moment of the at least one first solvent is about 20% or more greater than the dipole moment of the at least one second solvent. In various examples, the at least one first solvent is chosen from dichloromethane, tetrahydrofuran (THF), pyridine, trifluoroethanol, acetone, ethanol, methanol, N,N-Dimethylformamide, dimethyl sulfoxide (DMSO), isopropanol, water, ethyl acetate, trifluoroacetic acid, 1,1,1,3,3,3-hexafluoroisopropanol, 1-butanol, 1,2-dichloroethane, acetic acid, diglyme, acetonitrile, and the like, and any combination thereof. In various examples, the at least one second solvent is chosen from cyclohexane, hexane, benzene, toluene, dioxane, diethyl ether, chloroform, anisole, triethylamine, heptane, xylene, and the like, and any combination thereof. In various examples, the volume ratio of the at least one first solvent to the at least one second solvent is from about 1:99 to about 99:1, including all integer volume ratio values and ranges therebetween. In various examples, for each axial direction of the fibrous product, adjacent fibers of different layers are aligned (e.g., aligned one over the other) and are vertically stacked.

A method can achieve a desired level of fiber stacking using various fluid stock(s) comprising at least one first solvent having various dipole moment(s). In various examples, the fluid stock(s) comprise(s) at least one first solvent having a dipole moment of from about 1.5 D to about 4.2 D, including all 0.1 D values and ranges therebetween. In various examples, the at least one first solvent is chosen from dichloromethane, tetrahydrofuran (THF), pyridine, trifluoroethanol, acetone, ethanol, methanol, N,N-Dimethylformamide, dimethyl sulfoxide (DMSO), isopropanol, water, ethyl acetate, trifluoroacetic acid, 1,1,1,3,3,3-hexafluoroisopropanol, 1-butanol, 1,2-dichloroethane, acetic acid, diglyme, acetonitrile, and the like, and any combination thereof. In various examples, for each axial direction of the fibrous product, adjacent fibers of different layers are aligned (e.g., aligned one over the other) and are vertically stacked.

A method can achieve a desired level of fiber stacking using various fluid stock(s) having various conductivit(ies). In various examples, the fluid stock(s) further comprise(s) a conductive agent. In various examples, the conductive agent is chosen from a salt, a conductive polymer, and the like, and any combination thereof. In various examples, the salt is present in the fluid stock(s) at from about 0.01 weight % to about 10 weight %, including all 0.1 weight % values and ranges therebetween, based on the total weight of the material(s), or where the conductive polymer is present in the fluid stock(s) at from about 0.1 weight % to about 100 weight %, including all 0.1 weight % values and ranges therebetween, based on the total weight of the material(s). In various examples, for each axial direction of the fibrous product, adjacent fibers of different layers are aligned (e.g., aligned one over the other) and are vertically stacked.

A method can achieve a desired level of fiber fusion by applying various electric potentials to the nozzle(s). In various examples, the electric potential applied to the nozzle(s) is from about 50V to about 8 kV, including all 0.1 kV values and ranges therebetween. In various examples, for each axial direction of the fibrous product, adjacent fibers of different layers are aligned (e.g., one over the other) and are vertically stacked.

A method can form a fibrous product comprising one or more fiber(s) arranged in various predetermined patterns. In various examples, the fibrous product comprises one or more layer(s) each comprising one or more group(s) of fibers optionally aligned in one or more axial direction(s) of the fibrous product within and/or between the layer(s). In various examples, the group(s) of fibers is/are uniaxially, biaxially, or multi-axially oriented within and/or between the layer(s). In various examples, each group of fibers has a substantially constant winding angle, relative to the longitudinal axis of the fibrous product. In various examples, the substantially constant winding angle comprises an angle between from about 1° to about 89°, including all 0.1° values and ranges therebetween, relative to the longitudinal axis of the fibrous product. In various examples, the substantially constant winding angle is from about 1° to about 89°, including all 0.1° values and ranges therebetween, relative to the longitudinal axis of the fibrous product.

A method can use a power source providing various electrical potential(s) to the nozzle(s). In various examples, the electric potential is from about 50V to about 8 kV, including all 0.1 kV values and ranges therebetween.

A method can use various fluid stock(s). In various examples, the volume ratio of the at least one first solvent to the at least one second solvent is from about 1:99 to about 100:0, including all integer volume ratio values and ranges therebetween.

A fluid stock(s) can comprise various material(s). In various examples, the one or more material(s) comprise at least one polymer. In various examples, the at least one polymer comprises at least one biocompatible polymer, at least one biodegradable polymer, or the like, or any combination thereof. In various examples, the at least one polymer is thermo-reactive at a temperature of at least about 60° C. In various examples, the at least one polymer is chosen from a polyester, polyurethane, polyether, polyketal, polyamide, polyimide, polycarbonate, polyacrylate, polysaccharide, and the like, and any combination thereof. In various examples, the at least one polymer is chosen from polyglycolide or a polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxy valerate) (PHBV), polyethylene terephthalate (PET), polybutylene terephthalate (PBT), polyethylene glycol (PEG), polytrimethylene terephthalate (PTT), polyethylene naphthalate (PEN), poly(glycerol-sebacate) (PGS), palmitate functionalized poly(glycerol sebacate (PGSP), poly(epsilon caprolactone) (PCL), polymethyl methacrylate (PMMA), chitosan, gelatin, cellulose, polyvinyl alcohol (PVA), polyvinyl pyrrolidone (PVP), polydioxanone, derivatives thereof, and the like, and any combination thereof. In various examples, the at least one polymer has a concentration in the fluid stock(s) of from about 5% to about 90% w/V, including all 0.1% w/V values and ranges therebetween.

A fluid stock or fluid stocks can be used to form fiber(s) comprising various material(s). In various examples, the fluid stock(s) comprising the at least one polymer is/are ejected from the nozzle(s) to form one or more fiber(s) comprising the at least one polymer. In various examples, at least one fluid stock comprises at least one first polymer and at least one second polymer, and/or where at least a first fluid stock comprises at least one first polymer and at least one second fluid stock comprises at least a second polymer. In various examples, the fluid stock(s) comprising the at least one first polymer and the at least one second polymer are ejected from the same or different nozzle(s) to form one or more fiber(s) comprising the at least one first polymer and/or the at least one second polymer.

A fluid stock or fluid stocks can comprise various additive(s). In various examples, the fluid stock(s) further comprise(s) at least one additive. In various examples, the at least one additive is chosen from a therapeutic agent, a dye, an indicator agent, a drug, and the like, and any combination thereof. In various examples, the at least one additive is dissolved in or dispersed as particles in the fluid stock(s).

A method can form a fibrous product comprising various morphological and/or structural feature(s). In various examples, the fibrous product has an inner diameter of from about 0.5 mm to about 300 mm, including all 0.01 mm values and ranges therebetween, and/or an outer diameter of from about 0.51 mm to about 300 mm, including all 0.01 mm values and ranges therebetween. In various examples, the average diameter of the fibers is from about 100 nm to about 500 microns including all 1 nm values and ranges therebetween.

In various examples, the method further comprises one or more time(s) during formation of the fiber(s) one or more or all of the following: altering the volume ratio of the at least one first solvent to the at least one second solvent in the fluid stock(s); adding at least a third solvent to the fluid stock(s); altering the concentration of a conducting agent in the fluid stock(s); and altering the electric potential(s) applied to the nozzle(s), where fiber fusion, fiber stacking, or a combination thereof is altered.

In an aspect, the present disclosure provides products. In various examples, a product is made using a system and/or by a method of the present disclosure. In various examples, the product comprises one or more layer(s) of fibers. In various examples, the fibers are arranged in a predetermined pattern; the average diameter of the fibers is from about 100 nm to about 500 microns, including all 1 nm values and ranges therebetween; and the product comprises a desired fiber fusion and/or fiber stacking.

A product can comprise various types and degrees of desired fiber fusion and/or fiber stacking. In various examples, the fibrous product comprises a plurality of fusion points between respective portions of at least two intersected fibers, such that for each fusion point, a bottom surface of a first fiber is bonded to a top surface of a second fiber. In various examples, the plurality of fusion points between adjacent intersected fibers is observed at an average frequency of from about 5% to about 99%, including all 1% values and ranges therebetween. In various examples, the fibers comprise a plurality of fusion points between two intersected fibers, such that for each fusion point, a bottom surface of a first fiber is bonded to a top surface of a second fiber. In various examples, the fibers comprise an average frequency of fusion points from about 5% to about 99%, including all 1% values and ranges therebetween. In various examples, for each axial direction of the product, the adjacent fibers in different layers are aligned one over the other and are vertically stacked or vertically staggered.

A product can comprise various predetermined patterns. In various examples, each layer comprises one or more group(s) of fibers optionally aligned in one or more axial direction(s) of the product within and/or between the layer(s). In various examples, the group(s) of fibers is/are uniaxially, biaxially, or multi-axially oriented within and/or between the layer(s). In various examples, each group of fibers has a substantially constant winding angle. In various examples, the substantially constant winding angle comprises an angle between from about 1° to about 89°, including all 0.1° values and ranges therebetween, relative to the longitudinal axis of the fibrous product. In various examples, the winding angle is from about 1° to about 89°, including all 0.1° values and ranges therebetween.

In various examples, the predetermined pattern of fibers defines in the product a plurality of pores extending at least partially through the product. In various examples, the product comprises a plurality of pores. In various examples, the average width of the pores is at least 1 micron. In various examples, the pores are characterized by a cross-sectional shape in the form of a cube, a cuboid, a rhombohedron, or a rhomboid. In various examples, the pores have a cube shape, a cuboid shape, a rhombohedron shape, a rhomboid shape, or the like.

A product can comprise fiber(s) comprising various material(s). In various examples, each fiber comprises one or more material(s) which is/are thermo-reactive at a temperature of at least 60° C. In various examples, the one or more material(s) comprise(s) at least one polymer. In various examples, the at least one polymer comprises at least one biocompatible polymer, at least one biodegradable polymer, or the like, or any combination thereof. In various examples, the at least one polymer is thermo-reactive at a temperature of at least about 60° C. In various examples, the at least one polymer is chosen from a polyester, polyurethane, polyether, polyketal, polyamide, polyimide, polycarbonate, polyacrylate, polysaccharide, and any combination thereof. In various examples, the at least one polymer is chosen from polyglycolide or a polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxy valerate) (PHBV), polyethylene terephthalate (PET), polybutylene terephthalate (PBT), polyethylene glycol (PEG), polytrimethylene terephthalate (PTT), polyethylene naphthalate (PEN), poly(glycerol-sebacate) (PGS), palmitate functionalized poly(glycerol sebacate (PGSP), poly(epsilon caprolactone) (PCL), polymethyl methacrylate (PMMA), chitosan, gelatin, cellulose, polyvinyl alcohol (PVA), polyvinyl pyrrolidone (PVP), polydioxanone, derivatives thereof, and the like, and any combination thereof. In various examples, at least one fiber comprises at least one first polymer and at least one second polymer, and/or where at least one first fiber comprises at least one first polymer and at least second fiber comprises at least one second polymer.

In various examples, at least one fiber further comprises at least one additive. In various examples, the at least one additive is chosen from a therapeutic agent, a dye, an indicator agent, a drug, and the like, and any combination thereof.

A product can comprise various morphological and/or structural feature(s). In various examples, the product has an inner diameter of from about 0.5 mm to about 300 mm, including all 0.01 mm values and ranges therebetween, and/or an outer diameter of from about 0.51 mm to about 300 mm, including all 0.01 mm values and ranges therebetween. In various examples, the product is a conduit, a web, a patch, a mat, a cuff, or the like. In various examples, the product comprises a shape of at least a portion of an organ, a vessel, a body part, or the like. In various examples, the product is a conduit, a web, a patch, a mat, or a cuff, comprising a shape of at least a portion of an organ, a vessel, or a body part, or the like.

A product can be designed for various medical applications. In various examples, the product is an implantable medical device, a scaffold of an artificial tissue, or the like. In various examples, the product is an arteriovenous graft. In various examples, the arteriovenous graft comprises a first orifice and a second orifice. In various examples, the first orifice comprises an inner linear dimension that is from about 10% to about 1000%, including all 0.1% values and ranges therebetween, larger than an inner linear dimension of the second orifice. In various examples, the arteriovenous graft comprises a first end and a second end. In various examples, the first end comprises an inner diameter and the second end comprises an inner diameter, and the ratio of first end inner diameter to second end inner diameter is from about 1.5:1 to about 10:1, including all inner diameter ratio values and ranges therebetween, and/or the ratio of first end wall thickness to second end wall thickness is from about 1:1.25 to about 1:100 including all wall thickness ratio values and ranges therebetween.

In an aspect, the present disclosure provides solution electrowriting systems. A system can have various combinations of components and/or configurations. In various examples, a system comprises: a plurality of nozzles; a material supply system comprising one or more reservoir(s) fluidically coupled to the nozzles and configured to supply one or more fluid stock(s) to the nozzles thereby ejecting one or more jet stream(s) of the fluid stock(s) from the nozzles; a collector system configured to collect one or more fiber(s) formed by the jet stream(s) ejected from the nozzles; and one or more power source(s) configured to provide one or more electric potential(s) to each of the nozzles and, optionally, to the collector system, thereby providing one or more electric potential difference(s) between the collector system and each of the nozzles.

A system can comprise various nozzle arrangements. In various examples, the plurality of nozzles comprises one or more array(s) of nozzles. In various examples, the one or more array(s) of nozzles comprise(s) a linear array of nozzles, a radial array of nozzles, or the like, or any combination thereof. In various examples, the nozzles have a tip-to-tip separation distance of from about 1 mm to about 300 mm, including all 0.1 mm values and ranges therebetween. In various examples, the plurality of nozzles comprise a first nozzle or a first array of nozzles configured to form a group of fibers aligned in a first direction, and a second nozzle or a second array of nozzles configured to form a group of fibers aligned in a second direction, where the first direction and the second direction form an angle with a degree of from about 0° to about 90°, including all 0.1° values and ranges therebetween.

In various examples, the system further comprises a motorized stage configured to move one or more or all of the nozzles or one or more array(s) of the nozzles parallel to the longitudinal axis of the collector system during the electrowriting; and/or where one or more or all of the nozzles or one or more array(s) of the nozzles is/are configured to move parallel to the longitudinal axis of the collector system during electrowriting.

A system can comprise various collector system arrangements. In various examples, the collector system is positioned at a distance from the nozzles of from about 500 microns to about 50 mm, including all 1 micron values and ranges therebetween.

### BRIEF DESCRIPTION OF THE FIGURES

For a fuller understanding of the nature and objects of the disclosure, reference should be made to the following detailed description taken in conjunction with the accompanying figures herein.

FIGS. 1A-1F show solution electrowriting with palmitate functionalized poly(glycerol sebacate)/polyethylene terephthalate (PGSP/PET) and polycaprolactone (PCL). A diagram representation of a solution electrowriting device setup (FIG. 1A) with important fabrication parameters highlighted (AV—applied voltage), spinneret translational speed (VT), mandrel rotational speed (VR), and winding angle (w) defined. A solution electrowritten PGSP/PET conduit with 8 millimeter (mm) inner diameter (FIG. 1B) after thermal cure and removal from collection mandrel. (FIGS. 1C-1D) Scanning electron microscope (SEM) images of a solution electrowritten PGSP/PET conduit (FIG. 1C), and a solution electrowritten PCL conduit (FIG. 1D) (large image scale bars are 1 mm). Insets show high magnification images of fiber crossing points with fiber fusion present (FIG. 1C) or absent (FIG. 1D) (inset scale bars are 10 micron (μm)). (FIGS. 1E-1F) Effects of anisole inclusion into an electrowriting solution on PCL fiber fusion (FIG. 1E) and stacking height (in μm). (FIG. 1F). Inset in (FIG. 1E) shows PCL fiber fusion induced by anisole inclusion (scale bar is 3 μm). **p<0.01, ***p<0.001 compared to pure 1,1,1,3,3,3-hexafluoroisopropanol (HFIP) solution electrowriting in both FIG. 1E and FIG. 1F via post-hoc Tukey's Honestly Significant Difference (HSD).

FIGS. 2A-2E show applied voltage effects on fiber stacking. (FIGS. 2A-2D) SEM images of solution electrowritten PCL conduits fabricated from a 25% mass/Volume (m/V) PCL in HFIP solution with AV of (FIG. 2A) 1.33 kiloVolts (kV), (FIG. 2B) 2 kV, (FIG. 2C) 2.66 kV, and (FIG. 2D) 3.33 kV. (FIG. 2E) Quantification of fiber stacking height (in μm) with respect to applied voltage. All scale bars in large images are 5 mm, scale bars in image insets are 200 μm. *p, 0.05, **p<0.01, ***p<0.001 compared to AV of 1.33 kV via post-hoc Tukey's HSD.

FIGS. 3A-3B show a diagram representation of (FIG. 3A) a side view of stacked fibers and (FIG. 3B) a top view of stacked fibers.

FIGS. 4A-4B show a diagram representation of (FIG. 4A) a side view of distributed fibers and (FIG. 4B) a top view of distributed fibers.

FIGS. 5A-5I show fiber winding angle tunability. (FIGS. 5A-5C) Optical imaging of PCL fibers aligned in the (FIG. 5A) axial direction and (FIG. 5B) circumferential directions, and (FIG. 5C) of a ‘cage’ design consisting of alternating layers thereof. (FIGS. 5D-5F). Optical imaging (FIG. 5D) and SEM imaging (FIG. 5E) of PCL fibers with helical orientation. (FIG. 5F) Optical imaging of solution electrowritten PCL conduits with helical orientation deposited at different angles. (FIGS. 5G-5I) SEM images of PGSP/PET fibers with w of (FIG. 5G) 35°, (FIG. 5H) 45°, and (FIG. 5I) 75°. All scale bars are 1 mm.

FIG. 6 shows a solution electrowriting device prepared by modifying the motorized stage of a conventional horizontal electro-spinning device to enable solution electrowriting. (1) Syringe filled with electro-spinning solution, (2) High voltage power supply attached to spinneret (syringe tip), (3) Rotating mandrel with fibers collected, (4) Electro-spinning motor, (6) Syringe pump mounted onto (5) Motorized positioning stage to enable controllable spinneret translation parallel to rotating mandrel.

FIG. 7 shows PCL fibers solution electrowritten onto mandrels of various sizes. Mandrel sizes from left to right: 0.64 mm, 1.65 mm, 3 mm, 4.76 mm, and 25.6 mm.

FIGS. 8A-8C show methods to scale up solution electrowriting. (FIG. 8A) Double spinneret (syringe) approach to increases fabrication rate, and/or simultaneously spin two different solutions. (FIG. 8B) Custom built 6-needle spinneret used to increase fiber deposition rate 6-fold and improve fabrication times while maintaining high reproducibility (arrow points to individual fibers emerging from the tip of each spinneret). (FIG. 8C) Prototype drawing of a radial spinneret array.

FIG. 9 shows proton nuclear magnetic resonance (NMR) analysis that quantifies the actual palmitate content of PGSP. The integral area ratio of proton H a to H e is used for the quantification according to Equation 1.

FIG. 10 shows SEM images (Left—top view, Right—side view) of hybrid solution electrowritten—solution electrospun conduit. The conduit was fabricated by solution electrowriting of a 40% mass/Volume (m/V) solution of PGSP/PET in HFIP with AV of 1.2 kV, followed by solution electro-spinning of a 12% m/V solution of gelatin in trifluoroethanol (TFE) with AV of 5 kV, to form a gelatin nanofiber sheath encasing a PGSP/PET microfiber conduit.

FIG. 11 shows an in vivo transformation of an elastic biodegradable graft specifically designed for hemodialysis access. An arterial end gradually transitions to a venous end with an increase in diameter and decrease in wall thickness (an arteriovenous graft). Compliance of the venous end is designed to match that of the native vein.

FIG. 12 shows a transition zone of a graft designed for the specific demand of hemodialysis access considering the inherent differences of arteries and veins. Differences of the thickness and diameter of the two ends can be easily programmed.

FIGS. 13A-13M show control of fiber winding angle, spacing, and diameter of a graft fabricated using solution electrowriting. (FIG. 13A) Schematic of fiber winding angle (w). (FIGS. 13B-13D) Control of fiber winding angle. (FIGS. 13B-13C) SEM imaging of rat-sized graft; (FIG. 13D) SEM imaging of sheep-sized. Scale bars: 500 μm. (FIGS. 13E-13G) Control of fiber spacing to alter pore size. Scale bars: 1 mm. Inset scale bars: 30 μm. (FIG. 13H-13J) Control of fiber diameter. Scale bars: 2 μm. (FIG. 13K) Idealized architecture of an artery (© Mechanical Properties of Arteries: Identification and Application). (FIGS. 13L-13M) Polarized light micrographs revealing alignment of collagen fibers in (FIG. 13L) intima and (FIG. 13M) media; picrosirius red-staining, crossed polars. Scale bars: 100 μm.

FIGS. 14A-14E show a prototype of the transition zone of an arteriovenous graft (shown in FIG. 12) comprising a PGSP/PET conduit with a tapered inner diameter fabricated by solution electrowriting using a collection mandrel with a tapered diameter. (FIG. 14A) Side view. (FIGS. 14B-14C) Narrow end views. (FIGS. 14D-14C) Wide end views.

FIGS. 15A-15C shows: (FIG. 15A) An arteriovenous graft comprising a PGSP/PET conduit with a tapered inner diameter fabricated by solution electrowriting using a collection mandrel with a tapered diameter. Side view shows narrow end (0 mm) and wide end (50 mm) locations on the arteriovenous graft. (FIG. 15B) Plot of variation in inner diameter and wall thickness with various locations on the arteriovenous graft. (FIG. 15C): Scanning electron microscope (SEM) images show whole mount view of rings at various locations of the graft (top). Optical micrographs focusing on the top of the ring reveal differences in wall thickness (bottom).

FIGS. 16A-16B show transformation of PGS-PCL grafts in Lewis rat aorta. (FIG. 16A). H&E staining of graft transformation over time. Initial inflammation is largely resolved by 1 month. Scale bars top=250 μm, bottom=50 μm. (FIG. 16B). staining of vascular extracellular matrix molecules (Verhoeff for elastin, Masson's trichrome for collagen, and Safranin-O for glycosaminoglycan) at day 90. Scale bars=50 μm. Insets show grafts at day 0 and 365, where black sutures marked anastomoses. Staining of age-matched native aorta provided for comparison. *Top clipped while removing vena cava.

FIGS. 17A-17F show a PGS-PCL graft vs. a vein graft in Sprague Dawley carotid. PGS-PCL: synthetic graft, Vein: vein graft, Carotid: native common carotid artery. (FIG. 17A) Gross appearance of the grafts at day 0 and 90 post-implantation. Arrows mark sutures. (FIG. 17B) Representative ultrasound images of grafts and native artery at day 90. B-mode (top), color Doppler and pulse wave modes (bottom). Arrows mark suture lines. (FIG. 17C) Survival plot showing the overall patency of the grafts. There is no statistical difference between two grafts. P=0.15, log-rank test. (FIG. 17D) Representative images for immunofluorescence staining with the monocyte recruitment marker CCR2. Arrows mark CCR2⁺ cells. L, lumen. (FIG. 17E) Quantification of CCR2 staining. (FIG. 17F). The area ratio of iNOS⁺ to CD206+ cells representing the M1/M2 macrophage ratio. Data in E and F are means±SD (n=4). *P<0.05, Student's t-test.

FIG. 18 shows a biaxial inflation device used with a multiphoton microscope to image collagen fibers of grafts retrieved at day 90 (left). Planar images were obtained starting from the outer wall, moving down to the inner wall with images stacked to obtain projection images of collagen fibers across the thickness of the sample. Scale bars: 100 μm. Note the difference in collagen fiber density, morphology, and orientation between samples (right). Remodeled PGS-PCL grafts and carotid artery show collagen fiber recruitment, i.e. orientation changes upon loading, consistent with the collagen fibers being load bearing. In contrast, collagen fibers in the “arterialized” vein grafts are more disorganized and show no recruitment upon loading.

FIGS. 19A-19C show a sheep carotid interposition model. (FIG. 19A) Graft immediately after unclamping and hemostasis. Inset: B-mode and color Doppler image of graft at 15-day postimplantation. (FIG. 19B) Transverse H&E sections of graft explanted at 15 day. 4X view. Tears in inner and outer surfaces are cryosectioning artifacts. Graft material is marked by dotted lines. Scale bar: 500 μm. (FIG. 19C) 10X view. Closer examination of the wall, highlighting concentrations of inflammatory cells at the margins of the material. Graft material marked by dotted lines. Solid arrowheads point out some of many capillaries infiltrating the graft material. The perfusion is associated with inflammation, simultaneously it accelerates host cell infiltration and integration of the synthetic graft with the host. Scale bar: 100 μm.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise stated, “about,” “approximately,” “substantially,” or the like, when used in connection with a measurable variable such as, for example, a parameter, an amount, a temporal duration, or the like, are meant to encompass variations of, for example, a specified value including, for example, those within experimental error (which can be determined by for example, a given data set, an art accepted standard, and/or with a given confidence interval (e.g. 90%, 95%, or more confidence interval from the mean), such as, for example, variations of +/−10% or less, +/−5% or less, +/−1% or less, and +/−0.1% or less of and from the specified value), insofar such variations are appropriate to perform in the context of the disclosure. As used herein, unless otherwise stated, the terms “about,” “approximate,” “at or about,” and “substantially” can mean that the amount or value in question can be the exact value or a value that provides equivalent results or effects as recited in the sample claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error, and the like, and other factors known to those of skill in the art such that, for example, equivalent results, effects, or the like are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also, unless otherwise stated, include individual values (e.g., about 1%, about 2%, about 3%, and about 4%) and the sub-ranges (e.g., about 0.5% to about 1.1%; about 0.5% to about 2.4%; about 0.5% to about 3.2%, and about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “biodegradable polymer” describes a polymer that can be cleaved either enzymatically or hydrolytically to break it down sufficiently so as to allow the body to absorb or clear it away. A biodegradable graft is a graft in which at least a significant portion (e.g., at least 50%) of the graft degrades within one year of implantation.

As used herein, unless otherwise stated, the terms “coating”, “coatings”, “coated” and “coat” are forms of the same term defining material and process for making a material where a first substance or substrate surface is at least partially covered or associated with a second substance. Both the first and second substance are not required to be different. Further, when a surface is “coated” as used herein, unless otherwise stated, the coating can be effectuated by any chemical or mechanical bond or force, including linking agents. The “coating” need not be complete or cover the entire surface of the first substance to be “coated”. The “coating” may be complete as well (e.g., approximately covering the entire first substance). There can be multiple coatings and multiple substances within each coating. The coating may vary in thickness or the coating thickness can be substantially uniform. Coatings contemplated in accordance with the present disclosure include, but not limited to medicated coatings, drug-eluting coatings, drugs or other compounds, pharmaceutically acceptable carriers and any combination thereof, or any other organic, inorganic or organic/inorganic hybrid materials. In an example, the coating is a thromboresistant coating which has anticoagulant properties, such as, for example, heparin or the like, or any combination thereof.

As used herein, unless otherwise stated, the term “scaffold” describes a structural support facilitating cell infiltration and attachment in order to guide vessel growth. In some examples, a scaffold is biodegradable, bioresorbable, or the like, or any combination thereof. In some examples, a scaffold is a biodegradable polymer (e.g., polyester) scaffold. In some examples, a scaffold is used to form a vascular graft.

As used herein, unless otherwise stated, the term “vascular graft” describes a tubular member which acts as an artificial vessel. A vascular graft can include a single material, a blend of materials, a weave, a laminate or a composite of two or more materials.

As used herein, unless otherwise stated, the term “aligned” or “aligned fibers” or “aligned nozzles” describes a set of elements (e.g., fibers, nozzles) which have a parallel arrangement along one or more axial directions.

As used herein, unless otherwise stated, the term “layer” describes a region of continuous fiber or groups of continuous fibers traversing the perimeter of the fibrous product and the length of the fibrous product formed during a single pass of the spinneret of a solution electrowriting system along the length of the collector system. Alternately, the term “layer” can be describe a region of continuous fiber or groups of continuous fibers in a fibrous product at a substantially constant distance from the inner wall of the fibrous product. As used herein, unless otherwise stated, a layer can be further defined by structural and/or compositional features including fiber angle, degree of fiber fusion, degree of fiber stacking, degree of porosity, average pore width, shape of pores, fiber diameter, conduit wall thickness, fiber material(s), fiber additive(s), etc. As used herein, unless otherwise stated, a static layer has substantially the same structural and/or compositional features along the length the fibrous product. As used herein, unless otherwise stated, a dynamic layer has variable structural and/or compositional features along the length of the fibrous product. A dynamic layer may have two or more distinct regions along the length the fibrous product, each having substantially different structural and/or compositional features. The two or more distinct regions may vary continuously along the longitudinal axis of the fibrous product.

As used herein, unless otherwise stated, the term “nozzle” is used interchangeably with “spinneret” (e.g., the needle of a syringe, or the like). As used herein, unless otherwise stated, an “array” of nozzles comprises a plurality of the nozzles each arranged in a pattern and aligned with respect to one another in one or more axial dimension(s).

As used herein, unless otherwise stated, the term “power source” is used interchangeably with “high voltage power supply” and the term “electric potential” is used interchangeably with “voltage”.

As used herein, unless otherwise stated, the term “reservoir” is used interchangeably with a “container” (e.g., a syringe, a pump, a mixing chamber, a syringe pump, or the like). As used herein, unless otherwise stated, the term “fluid stock” can be a solution, a suspension, an emulsion, or the like.

As used herein, unless otherwise stated, the term “polar solvents” are solvents having larger dipole moments (or partial charge) due to the larger electronegativity difference between the bonded atoms (such as, for example, between oxygen and nitrogen, or the like) and the shape and geometry of the molecule. As used herein, unless otherwise stated, the term “non-polar solvents” are solvents having smaller dipole moments (or partial charge) due to the smaller electronegativity difference between the bonded atoms (such as, for example, between carbon and hydrogen, or the like) and the shape and geometry of the molecule. Solvent polarity increases with increasing dipole moment. As used herein, unless otherwise stated, a non-polar solvent will have a dipole moment in the range of 0-1.5 Debye units (D), while a polar solvent will have a dipole moment in the range of 1.5-4.2 D. Solvent polarity can alternately be measured the dielectric constant (or permittivity) of a solvent, which similarly increases with solvent polarity.

As used herein, unless otherwise stated, the term “thermo-reactive” refers to materials which react (e.g., denature, degrade, crosslink, or otherwise change, or any combination thereof) at a specified temperature. Non-limiting examples of thermo-reactive materials include thermosetting materials, and thermo-degradable polymers (e.g., polymers which undergo thermal depolymerization, chain scission, removal of polymer side groups, polymer oxidation, or the like, or any combination thereof). Non-limiting examples of thermo-reactive materials include proteins, growth factors and cytokines: vascular endothelial growth factors (VEGF), nerve growth factor (NGF), brain derived neurotrophic factor (BDNF), epidermal growth factor (EGF), fibroblast growth factors (FGF), transforming growth factors (TGF-β1, TGF-β2, TGF-β3), interleukins (IL-1-17), ephrins, colony stimulating factors (CSF), bone morphogenic proteins (BMP), neurotrophin-3 (NT-3), platelet derived growth factor (PDGF), and the like, and any combination thereof. Non-limiting examples of thermo-reactive additives (e.g., thermo-degradable additives, or the like) include pharmaceutical compounds and temperature indicators. Non-limiting examples of thermo-reactive pharmaceutical compounds include rapamycin, tamoxifen, acetaminophen, ibuprofen, diclofenac, paclitaxel, amoxicillin, gentamicin, doxorubicin, cyclodextrin, naproxen, indomethacin, ketoprofen, metronidazole, ciprofloxacin, curcumin, insulin, lovastatin, and other drugs used as antimicrobials, anti-inflammatory agents, antineoplastics, statins, and the like, and any combination thereof. Non-limiting examples of thermo-reactive additives include indicators and the like, and any combination thereof. Non-limiting examples of thermo-reactive indicators include fluorescein isothiocyanate (FITC), rhodamine b, and the like and any combination thereof.

The present disclosure provides solution electrowriting systems. The present disclosure also provides solution electrowriting methods, and products made using the systems or by the methods.

Multiple additive manufacturing techniques have been developed in recent years to produce scaffolds with tunable physical, chemical, and mechanical properties. Solution electro-spinning, although an older and more established technique, normally cannot achieve the resolution and tunability of these newer manufacturing techniques. Described herein, inter alia, is electrospinning procedures that, for example, enable the control of fiber placements in addition to fiber diameter and spacing in the electrospun products. In various examples, near-field electrospinning, solvent electrospinning, and utilization of a rotating mandrel are combined to create 3D fibrous products, such as, for example, conduits and the like.

Solution electrowriting further enables users to create scaffolds incorporating drugs or other co-solutes into the polymer to further modify the physical, biological, or chemical properties of the scaffold. Solution electrowriting may be a better approach for patterned drug-delivering fibrous scaffolds compared to melt electrowriting. During melt electrowriting, the increased temperature needed to melt the polymer may damage the drug molecules rendering them ineffective. This could be avoided with a solution-based approach. This is also an appealing approach for drug delivery applications since it is reasonable to assume that any drug that is soluble in a polymer solution can be incorporated into a solution electrowritten scaffold. Non-limiting exemplary drugs that have been incorporated into electrospun scaffolds include: Minocycline, Fingolimod, Dexamethasone, Paclitaxel, Vancomycin, Riluzole, 6-Aminonicotinamide, Ibuprofen, Naproxen, Meloxicam, Ketoprofen, Acetaminophen, Loratadine, Ciprofloxacin, Doxorubicin, Tetracycline, and Acyclovir.

Near-field solvent electro-spinning also demonstrates higher fabrication resolution compared to both fused-deposition modeling (FDM) and stereolithography apparatus (SLA) 3D printing. This technology holds promise to control fiber diameter in the range from 0.5 to 20 which is up to 200-fold finer than state-of-the-art stereolithography can achieve.

Near-field solvent electro-spinning has the additional advantage of allowing orientation control of a single fiber, versus control only over the orientation of bundled fibers, which is currently achievable by braiding technology, a mature technology for controlling fiber orientation and commonly used to fabricate tubular conduits for clinical use.

Further, this disclosure differs from standard electro-spinning practice because, in various examples, electro-spinning is performed over a much shorter distance using a drastically lower applied voltage and far less polymer solution. For example, solution electro-writing has been successfully performed over collection distances ranging from about 400 micrometers (μm) up to about 30 mm. The programmable stage/platform as well as the decreases in solution flow rate, applied voltage, and fiber collection distance all allow for the previously described improvements in fiber morphology and pattern control.

In various examples, an existing standard electro-spinning setup was modified to include a stage/platform with programmable speed and motion. Then, by altering standard electro-spinning parameters and introducing code for the programmable stage/platform the user can easily electrowrite tubular conduits using this technique.

In an aspect, the present disclosure provides solution electrowriting systems. In various examples, the solution electrowriting systems include one or more array(s) of nozzles. Non-limiting examples of solution electrowriting systems are provided herein.

A system can have various combinations of components and/or configurations. In various examples, the system comprises: a plurality of nozzles; a material supply system comprising one or more reservoir(s) fluidically coupled to the nozzles and configured to supply one or more fluid stock(s) to the nozzles thereby ejecting one or more jet stream(s) of the fluid stock(s) from the nozzles; a collector system configured to collect one or more fiber(s) formed by the jet stream(s) ejected from the nozzles; and one or more power source(s) configured to provide one or more electric potential(s) to each of the nozzles and, optionally, to the collector system, thereby providing one or more electric potential difference(s) between the collector system and each of the nozzles.

A system can have a two-dimensional (2D) (e.g., flat) collector system or a three-dimensional (3D) (e.g., cylindrical) collector system. A system can comprise various nozzle arrangements between each nozzle, between the nozzles and the collector system, or any combination thereof. In various examples, the tip of each nozzle is oriented toward a surface of the collector system. In various examples, the plurality of nozzles comprises at least three nozzles. In various examples, the plurality of nozzles comprises one or more array(s) of nozzles. In various examples, the one or more array(s) of nozzles comprise(s) a linear array of nozzles, a radial array of nozzles, or the like, or any combination thereof. In various examples, the linear array of nozzles is aligned along the longitudinal axis of and oriented toward a surface of the collector system. In various examples, the radial array of nozzles is positioned around and oriented toward a surface of the perimeter (e.g., the circumference) of the collector system. In various examples, the nozzles have a tip-to-tip separation distance of from about 1 mm to about 300 mm, including all 0.1 mm values and ranges therebetween. In various examples, the same fluid stock is supplied to all nozzles. In various examples, different fluid stocks are supplied to all nozzles. In various examples, different fluid stocks are supplied to two or more nozzles. In various examples, different fluid stocks are supplied to two or more nozzles of one or more arrays of the nozzles (e.g., a linear array, a radial array, or the like, or any combination thereof).

In various examples, the plurality of nozzles comprise a first nozzle or a first array of nozzles configured to form a group of fibers aligned in a first direction, and a second nozzle or a second array of nozzles configured to form a group of fibers aligned in a second direction, where the first direction and the second direction form an angle with a degree of from about 0° to about 90°, including all 0.1° values and ranges therebetween. In various examples, the first direction and the second direction form an angle with a degree of from about 0° to about 90°, from about 15° to about 90°, from about 25° to about 80°, or more preferably from about 35° to about 75°, including all 0.1° values and ranges therebetween.

A system can comprise various material supply systems. In various examples, the material supply system is configured to deliver a single fluid stock or at least two fluid stocks from at least two reservoirs for at least one nozzle (such as, for example, delivering at least two fluid stocks into one nozzle or delivering different fluid stocks to different nozzles).

A system may comprise a motorized stage. In various examples, the system further comprises a motorized stage configured to move one or more or all of the nozzles or one or more array(s) of the nozzles parallel to the longitudinal axis of the collector system during the electrowriting; and/or where one or more or all of the nozzles or one or more array(s) of the nozzles is/are configured to move parallel to the longitudinal axis of the collector system during electrowriting. In various examples, the motorized stage moves at a speed of from about 0.5 cm/s to about 20 cm/s or preferably from about 1 cm/l to about 15 cm/s, including all 0.01 cm/s values and ranges therebetween.

A system may comprise a movable collector system. Non-limiting examples of a moving collector system include a rotating mandrel, a Modular Rotating Collector System, or a motorized stage configured to move the collector system. In various examples the movable collector system is a mandrel having a diameter of from about 0.5 mm to about 30 mm. In various examples, the mandrel has a uniform diameter or a tapered diameter along the length of the mandrel. In various examples, the movable collector system is a rotating mandrel having a rotation speed (VR) selected from about 0.5 cm/s to about 20 cm/s including all 0.01 cm/s values and ranges therebetween or preferably from about 1 cm/s to about 10 cm/s including all 0.01 cm/s values and ranges therebetween.

A system can comprise various distances between the collector system and the nozzles. In various examples, the collector system is positioned at a distance from the nozzles of from about 500 microns to about 50 mm, including all 1 micron values and ranges therebetween. In various examples, the collector system is positioned at a distance from the nozzles of less than about 50 mm, preferably in a range from about 500 microns to about 30 mm, including all 1 micron values and ranges therebetween or more preferably in a range from about 1 mm to about 20 mm, including all 1 micron values and ranges therebetween.

In various examples, this disclosure uses the same components as a standard electro-spinning device: syringe pump, power supply, and a rotating fiber collection mandrel. In various examples, a motorized stage/platform is used to program motion of the electro-spinning spinneret and/or the collection mandrel.

In various examples, a fluid stock described herein for the electrowriting systems and/or methods is used in conventional electrospinning systems and/or methods to improve the fiber fusion and/or achieve desirable fiber alignments.

In various examples, a system is a modified standard extrusion-based 3D printer. In various examples, the conventional extrusion print head is replaced with a syringe pump and power supply and the standard flat 3D printer sample deposition platform replaced with a rotating mandrel to create a device with very similar functionality. In various examples, a system is an extrusion-based 3D printer comprising a syringe pump, power supply, and a rotating mandrel.

In an aspect, the present disclosure provides solution electrowriting methods. In various examples, using particular components and/or conditions the methods provide fibrous products having desirable fiber fusion and/or fiber stacking. In various examples, a method is carried out using a system of the present disclosure. Non-limiting examples of solution electrowriting methods are provided herein.

Fibrous product length is tunable with the instant methods and is considered to be only limited by the range of the motorized stage translating the spinneret. Fibrous product inner diameter and wall thickness are also tunable with this technique and can be varied along the length of the conduit using mandrels of non-uniform diameters. Fiber thickness, fiber fusion, fiber stacking, and winding angle are also tunable and can be controlled by varying flow rate, applied voltage, spinneret translational speed mandrel rotation speed, or any combination thereof, along the length of the conduit and/or between translations of the spinnerets along the length of the conduit.

In various examples, a method comprises forming one or more fiber(s) from a jet stream, which is formed from one or more fluid stocks(s). In various examples, a method further comprises collecting the fiber(s) to form a fibrous product comprising one or more fiber(s) arranged in a predetermined pattern. In various examples, a method further comprises collecting the fiber(s) to form a fibrous product, where a desired fiber fusion and/or a desired fiber stacking is observed in the fibrous product. A fibrous product can have various predetermined patterns. In various examples, a predetermined pattern is a particular predetermined three-dimensional shape and/or a particular predetermined fiber orientation (e.g., fiber fusion, fiber stacking, winding angle, or any combination thereof).

In various examples, a method comprises providing a solution electrowriting system comprising: one or more nozzle(s); a material supply system comprising one or more reservoir(s) fluidically coupled to the nozzle(s) and configured to supply one or more fluid stock(s) to the nozzle(s) thereby ejecting one or more jet stream(s) of the fluid stock(s) from the nozzle(s); a collector system configured to collect one or more fiber(s) formed by the jet stream(s) ejected from the nozzle(s); and one or more power source(s) configured to provide one or more electric potential(s) to each of the nozzle(s) and, optionally, to the collector system, thereby providing one or more electric potential difference(s) between the collector system and each of the nozzle(s). In various examples, the method further comprises ejecting the fluid stream(s) of the fluid stock(s) from the nozzle(s) to form the fiber(s). In various examples, the method further comprises collecting the fiber(s) with the collector system to form a fibrous product comprising one or more fiber(s) arranged in a predetermined pattern. In various examples, the method further comprises releasing the fibrous product from collector system, where a desired fiber fusion and/or a desired fiber stacking is observed in the fibrous product.

A collector system may include a water-soluble sacrificial layer. In various examples, the collection system comprises a water-soluble sacrificial layer on the surface upon which the fibers are collected. In such cases, the releasing may comprise dissolving the sacrificial layer in water. In various examples, the sacrificial layer comprises a sodium hyaluronate (HA), polyvinyl alcohol, polyvinylpyrrolidone, gelatin, collagen, chitosan, glucose, sucrose, dextran, or the like, or any combination thereof.

A method may also include heating and/or drying the fibrous product. A method may also include irradiating the fibrous product with electromagnetic radiation (e.g., UV radiation, or the like, or any combination thereof), ionizing radiation (e.g., electron beam (EB) radiation, or the like, or any combination thereof), or the like, or any combination thereof. In various examples, the fibrous product is cured, crosslinked, or the like, or any combination thereof, by the heating, the irradiating, or the like, or any combination thereof. In various examples, drying comprises thermal drying (e.g., heating), thermal-vacuum drying, freeze-vacuum drying, air drying, fan drying, desiccant drying, or the like, or any combination thereof.

In various examples, a method further comprises, after the collecting and/or the releasing, heating and/or drying the fibrous product. In various examples, the heating comprises heating the fibrous product at a temperature from about 50° C. to about 160° C., from about 70° C. to about 150° C., or from about 80° C. to about 110° C., or at least about 60° C., at least about 70° C., at least about 80° C., at least about 90° C., or at least about 100° C., or at most about 200° C., at most about 170° C., at most about 150° C., or at most about 120° C.

A method can use a system comprising various fluid stock(s). In various examples, each fluid stock comprises a solution comprising at least one first solvent and, optionally, at least one second solvent, and one or more material(s) configured to form at least a portion of a fiber upon ejection of the jet stream(s) of the fluid stock(s) from the nozzle(s). In various examples, the material(s) is/are dissolvable in at least one of the solvent(s) to form a solution.

A method can achieve a desired level of fiber fusion using various fluid stock(s) comprising at least one first solvent and at least one second solvent having various boiling point(s). In various examples, the fluid stock(s) comprise at least one first solvent having a boiling point of less than about 80° C., and at least one second solvent having a boiling point of at least about 80° C. or greater. In various examples, the at least one first solvent has a boiling point of less than about 80° C. (or less than about 90° C., or less than about 75° C., or less than about 70° C., or less than about 65° C., or less than about 60° C., or less than about 55° C., or less than about 50° C., or less than about 45° C.); and the at least one second solvent has a lower volatility than the first solvent, wherein the at least one second solvent has a boiling point of at least about 80° C., at least about 90° C., at least about 100° C., at least about 110° C., at least about 120° C., at least about 130° C., at least about 140° C., or at least about 150° C.

A fluid stock may include at least one first solvent and at least one second solvent. In various examples, the fluid stock(s) comprise(s) at least one first solvent and at least one second solvent, where the boiling point of the at least one second solvent is from about 10° C. to about 200° C., including all 0.1° C. values and ranges therebetween, higher than the boiling point of the at least one first solvent. In various examples, the at least one first solvent has a high volatility, wherein the at least one second solvent has a lower volatility than the first solvent (e.g., the second solvent has a boiling point of at least about 10° C. higher (or at least about 20° C. higher, or at least about 30° C. higher, or at least about 40° C. higher, or at least about 50° C. higher, or at least about 60° C. higher, or at least about 70° C. higher, or at least about 80° C. higher, or at least about 90° C. higher, or at least about 100° C. higher, or at least about 110° C. higher) than the first solvent).

In various examples, the at least one first solvent is chosen from diethyl ether, dichloromethane (DCM), acetone, 1,1,1,3,3,3-hexafluoroisopropanol (HFIP), chloroform, methanol, tetrahydrofuran (THF), trifluoroethanol (TFE), ethanol, acetonitrile, cyclohexane, benzene, ethyl acetate, hexane, trifluoroacetic acid, isopropanol, and the like, and any combination thereof. In various examples, the at least one second solvent is chosen from water, dioxane, toluene, pyridine, N,N-dimethylformamide (DMF), anisole, dimethyl sulfoxide (DMSO), 1,2-dichloroethane, triethylamine, heptane, butanol, acetic acid, xylene, diglyme (diethylene glycol diethyl ether), and the like, and any combination thereof. In various examples, the volume ratio of the at least one first solvent to the at least one second solvent is from about 1:99 to about 99:1, including all integer volume ratio values and ranges therebetween. In various examples, the fibers comprise a plurality of fusion points between two intersected fibers, such that for each fusion point, a bottom surface of a first fiber is bonded to a top surface of a second fiber. In various examples, the plurality of fusion points between two intersected fibers is observed at an average frequency of from about 5% to about 99%, including all 0.1% values and ranges therebetween.

A method can achieve a desired level of fiber fusion using various fluid stock(s) comprising at least one first solvent having various boiling point(s). In various examples, the fluid stock(s) comprise(s) at least one first solvent having a boiling point of from about 70° C. to about 120° C., including all 0.1° C. values and ranges therebetween. In various examples, the at least one first solvent is chosen from trifluoroethanol (TFE), ethanol, acetonitrile, cyclohexane, benzene, ethyl acetate, hexane, trifluoroacetic acid, isopropanol, water, dioxane, toluene, pyridine, and the like, and any combination thereof. In various examples, the fibers comprise a plurality of fusion points between two intersected fibers, such that for each fusion point, a bottom surface of a first fiber is bonded to a top surface of a second fiber. In various examples, for at least one or more or all fusion points, the overlapped distance of the first fiber and the second fiber is less than about 15%, less than about 10%, less than about 5%, less than about 4%, less than about 3%, less than about 2%, or less than about 1% of the diameter of the first fiber and/or second fiber.

In various examples, the plurality of fusion points between two intersected fibers is observed at an average frequency of from about 5% to about 99%, including all values and ranges therebetween. In various examples, the fibers comprise an average frequency of fusion points of from about 5% to about 99%, or from about 5% to about 90%, or from about 10% to about 60%, or at least about 5%, or at least about 7%, or at least about 10%, or at least about 15%, or at least about 20%, or at least about 30%, or at most about 99%, or at most about 90%, or at most about 80%, or at most about 70%, or at most about 60%, or at most about 50%, including all 1% values and ranges therebetween.

A method can achieve a desired level of fiber stacking using various fluid stock(s) comprising at least one first solvent and at least one second solvent having various dipole moment(s). In various examples, the fluid stock(s) comprise(s) at least one first solvent and at least one second solvent, where the at least one first solvent has a dipole moment of from about 1.5 D to about 4.2 D, including all 0.1 D values and ranges therebetween, and the at least one second solvent has a dipole moment of from about 0 D to less than about 1.5 D, including all 0.1 D values and ranges therebetween. In various examples, the fluid stock(s) comprise(s) at least one first solvent and at least one second solvent, and where the dipole moment of the at least one first solvent is about 20% or more greater than the dipole moment of the at least one second solvent. In various examples, the at least one first solvent is chosen from dichloromethane, tetrahydrofuran (THF), pyridine, trifluoroethanol, acetone, ethanol, methanol, N,N-Dimethylformamide, dimethyl sulfoxide (DMSO), isopropanol, water, ethyl acetate, trifluoroacetic acid, 1,1,1,3,3,3-hexafluoroisopropanol, 1-butanol, 1,2-dichloroethane, acetic acid, diglyme, acetonitrile, and the like, and any combination thereof. In various examples, the at least one second solvent is chosen from cyclohexane, hexane, benzene, toluene, dioxane, diethyl ether, chloroform, anisole, triethylamine, heptane, xylene, and the like, and any combination thereof. In various examples, the volume ratio of the at least one first solvent to the at least one second solvent is from about 1:99 to about 99:1, including all integer volume ratio values and ranges therebetween. In various examples, for each axial direction of the fibrous product, adjacent fibers of different layers are aligned over one the other and are vertically stacked.

The method can achieve a desired level of fiber stacking using various fluid stock(s) comprising at least one first solvent having various dipole moment(s). In various examples, the fluid stock(s) comprise(s) at least one first solvent having a dipole moment of from about 1.5 D to about 4.2 D, including all 0.1 D values and ranges therebetween. In various examples, the at least one first solvent is chosen from dichloromethane, tetrahydrofuran (THF), pyridine, trifluoroethanol, acetone, ethanol, methanol, N,N-Dimethylformamide, dimethyl sulfoxide (DMSO), isopropanol, water, ethyl acetate, trifluoroacetic acid, 1,1,1,3,3,3-hexafluoroisopropanol, 1-butanol, 1,2-dichloroethane, acetic acid, diglyme, acetonitrile, and the like, and any combination thereof. In various examples, for each axial direction of the fibrous product, adjacent fibers of different layers are aligned over one the other and are vertically stacked.

The method can achieve a desired level of fiber stacking using various fluid stock(s) comprising at least one first solvent and optionally, at least one second solvent and can have various conductivit(ies). In various examples, the fluid stock(s) further comprise(s) a conductive agent. In various examples, the conductive agent is chosen from a salt, a conductive polymer, and the like, and any combination thereof.

Various salts can be used. Combinations of salts may be used. In various examples, the salt is chosen from: potassium iodide, potassium bromide, potassium chloride, sodium iodide, sodium bromide, sodium chloride, potassium fluoride, sodium fluoride, lithium iodide, lithium bromide, lithium chloride, lithium oxide, sodium hydride, lithium hydride, and the like, and any combination thereof. In various examples, the conductive polymer is chosen from: polypyrroles, polycarbazoles, polyindoles, polyazepines, polyanilines, polyfluorenes, polyphenylenes, polypyrenes, polyazulenes, polynapthalenes, polythiophenes, polyacetylenes, and the like, and any combination thereof.

In various examples, the salt is present in the fluid stock(s) at from about 0.01 weight % to about 10 weight %, including all 0.1 weight % values and ranges therebetween, based on the total weight of the material(s), or where the conductive polymer is present in the fluid stock(s) at from about 0.1 weight % to about 100 weight %, including all 0.1 weight % values and ranges therebetween, based on the total weight of the material(s). In various examples, for each axial direction of the fibrous product, adjacent fibers of different layers are aligned over one the other and are vertically stacked.

The method can achieve a desired level of fiber fusion using various fluid stock(s) comprising at least one first solvent and optionally, at least one second solvent and by applying various electric potentials can be applied to the nozzle(s). In various examples, the electric potential applied to the nozzle(s) is from about 50V to about 8 kV, including all values and ranges therebetween. In various examples, the electric potential applied to the nozzle(s) is selected from a range of from about 100V to about 8 kV, preferably a range of from about 100V to about 5000V, or more preferably a range of from about 1000V to about 5000V, or at least about 100 V, at least about 200 V, at least about 500 V, at least about 1 kV, or less than about 10 kV, less than about 8 kV, less than about 5 kV, less than about 4 kV, or less than about 3 kV, including all 0.1 kV values and ranges therebetween. In various examples, for each axial direction of the fibrous product, adjacent fibers of different layers are aligned over one the other and are vertically stacked.

The method can form a fibrous product comprising one or more fiber(s) arranged in various predetermined patterns. In various examples, the fibrous product comprises one or more layer(s) each comprising one or more group(s) of fibers optionally aligned in one or more axial direction(s) of the fibrous product within and/or between the layer(s). In various examples, the group(s) of fibers is/are uniaxially, biaxially, or multi-axially oriented within and/or between the layer(s). In various examples, each group of fibers has a substantially constant winding angle, relative to the longitudinal axis of the fibrous product. In various examples, the substantially constant winding angle is from about 1° to about 89°, including all 0.1° values and ranges therebetween, relative to the longitudinal axis of the fibrous product. In various examples, the fibrous product comprises two groups of aligned fibers aligned in two directions with a substantially constant winding angle from about 15° to about 90°, from about 25° to about 80° or more preferably from about 35° to about 75°, including all 0.1° values and ranges therebetween, relative to the longitudinal axis of the product.

The method can use a power source providing various electrical potential(s) to the nozzle(s). In various examples, the electric potential is from about 50V to about 8 kV, including all 0.1 kV values and ranges therebetween. In various examples, the electric potential applied to the nozzle(s) is selected from a range of from about 100V to about 8 kV, preferably a range of from about 100V to about 5000V, or more preferably a range of from about 1000V to about 5000V, or at least about 100 V, at least about 200 V, at least about 500 V, at least about 1 kV, or less than about 10 kV, less than about 8 kV, less than about 5 kV, less than about 4 kV, or less than about 3 kV, including all 0.1 kV values and ranges therebetween.

The method can use various fluid stock(s). In various examples, the volume ratio of the at least one first solvent to the at least one second solvent is from about 1:99 to about 100:0, including all integer volume ratio values and ranges therebetween. In various examples, the volume ratio of the at least one first solvent to the at least one second solvent is 100:0 or is from about 1:99 to about 99:1, from about 1:9 to about 9:1, from about 1:4 to about 4:1, or from about 1:3 to about 3:1, or is from at least about 1:99, about 1:9, about 1:8, about 1:7, about 1:6, about 1:5, about 1:4, about 1:3, about 1:2, or is about 1:1, or is at most about 99:1, about 9:1, about 8:1, about 7:1, about 6:1, about 5:1, about 4:1, about 3:1, about 2:1, or about 1:1, including all integer volume ratio values and ranges therebetween.

In various examples, the at least one first solvent is from about 10% to about 100%, including all 1% values and ranges therebetween, based on the volume of the total solvent, or from about 20% to about 80%, including all 1% values and ranges therebetween, based on the volume of the total solvent, or preferably from about 25% to about 75%, including all 1% values and ranges therebetween, based on the volume of the total. In various examples, the temperature of the fluid stock(s) is less than about 80° C., less than about 70° C., less than about 60° C., less than about 50° C., less than about 40° C., less than about 30° C. before, during and/or after the fluid stock(s) is/are ejected from the nozzle(s).

The fluid stock(s) can comprise various material(s). In various examples, the one or more material(s) comprise at least one polymer. In various examples, the at least one polymer comprises at least one biocompatible polymer, at least one biodegradable polymer, or the like, or any combination thereof. In various examples, the at least one polymer is thermo-reactive at a temperature of at least about 60° C. In various examples, the at least one polymer is thermo-reactive at a temperature of at least 60° C., at least 100° C., at least 150° C., at least 166° C., at least 170° C., at least 180° C., at least 190° C., at least 200° C., at least 210° C., at least 220° C., at least 230° C., at least 240° C., at least 250° C., at least 260° C., at least 270° C., at least 280° C., at least 290° C., or at least 300° C.

A fluid stock can comprise various polymers or combinations of polymers. In various examples, the at least one polymer comprises at least one prepolymer (e.g., a polymer precursor, or the like, or any combination thereof) comprising at least one reactive polymer and, optionally, at least one unreacted monomer, at least one polymerization catalyst, at least one crosslinking agent, or the like, or any combination thereof. In various examples, the at least one prepolymer is curable (e.g., polymerizable, crosslinkable, or the like, or any combination thereof) by heating, electromagnetic radiation curing (e.g., UV curing, or the like, or any combination thereof), ionizing radiation curing (e.g., electron beam (EB) curing), or the like, or any combination thereof), or the like, or any combination thereof. In various examples, the at least one polymer comprises a crosslinked polymer, hydrogel, or the like, or any combination thereof. In various examples, the at least one polymer does not comprise a crosslinked polymer, hydrogel, or the like, or any combination thereof.

In various examples, the at least one polymer, is a synthetic organic polymer, a natural organic polymer, or an inorganic polymer. In various examples, the at least one polymer is chosen from a polyester, polyurethane, polyether, polyketal, polyamide, polyimide, polycarbonate, polyacrylate, polysaccharide, and the like, and any combination thereof. In various examples, the at least one polymer is chosen from polyglycolide or a polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxy valerate) (PHBV), polyethylene terephthalate (PET), polybutylene terephthalate (PBT), polyethylene glycol (PEG), polytrimethylene terephthalate (PTT), polyethylene naphthalate (PEN), poly(glycerol-sebacate) (PGS), palmitate functionalized poly(glycerol sebacate (PGSP), poly(epsilon caprolactone) (PCL), polymethyl methacrylate (PMMA), chitosan, gelatin, cellulose, polyvinyl alcohol (PVA), polyvinyl pyrrolidone (PVP), polydioxanone, derivatives thereof, and the like, and any combination thereof. In various examples, the at least one polymer is chosen from: silk, gelatin, and a polysaccharide, such as, for example, cellulose, chitin, chitosan, hyaluronic acid, dextran, and alginate, and the like, and any combination thereof. In various examples, the at least one polymer is chosen from: polysilanes, polysiloxanes, polysulfides, polysilazanes, polyphosphazenes, and the like, and any combination thereof. In various examples, a polymer is or polymers are not a polymer or polymers than can melt electrospun (e.g., electrospun to provide a product).

In various examples, the materials comprise at least one first polymer and at least one second polymer. In various examples, the at least one first polymer is PGSP and/or the at least one second polymer is Polyethylene terephthalate (PET).

In various examples, the at least one polymer has a concentration in the fluid stock(s) of from about 5% to about 90% w/V, including all 0.1% w/V values and ranges therebetween. In various examples, the at least one polymer has a concentration in the fluid stock(s) of from about 5% to 50% w/V, preferably from about 15% to about 40% w/V, or more preferably from about 20% to about 30% w/V, including all 0.1% w/V values and ranges therebetween.

The fluid stock(s) can be used to form fiber(s) comprising various material(s). In various examples, the material supply system is configured to deliver a single fluid stock or at least two fluid stocks from at least two reservoirs for at least one nozzle (such as, for example, delivering at least two fluid stocks into one nozzle or delivering different fluid stocks to different nozzles). In various examples, the fluid stock(s) comprising the at least one polymer is/are ejected from the nozzle(s) to form one or more fiber(s) comprising the at least one polymer. In various examples, at least one fluid stock comprises at least one first polymer and at least one second polymer, and/or where at least a first fluid stock comprises at least one first polymer and at least one second fluid stock comprises at least a second polymer. In various examples, the fluid stock(s) comprising the at least one first polymer and the at least one second polymer are ejected from the same or different nozzle(s) to form one or more fiber(s) comprising the at least one first polymer and/or the at least one second polymer.

The fluid stock(s) can comprise various additive(s). In various examples, the fluid stock(s) further comprise(s) at least one additive. In various examples, the at least one additive is chosen from a therapeutic agent, a dye, an indicator agent, a drug, and the like, and any combination thereof. In various examples, the at least one additive is dissolved in or dispersed as particles in the fluid stock(s).

The method can form a fibrous product comprising various morphological and/or structural feature(s). In various examples, the fibrous product has an inner diameter of from about 0.5 mm to about 300 mm, including all 0.01 mm values and ranges therebetween, and/or an outer diameter of from about 0.51 mm to about 300 mm, including all 0.01 mm values and ranges therebetween. In various examples, the average diameter of the fibers is from about 100 nm to about 500 microns including all 1 nm values and ranges therebetween.

A method may be a static method. In various examples, the features of a static method (e.g., steps, components, conditions, parameters, or the like, any combination thereof) are substantially constant (or constant) during substantially all (or all) of the method (or method steps). A static method may be used to fabricate a fibrous product comprising one or more static layer(s). A method may be a dynamic method. In various examples, in a dynamic method, at least one of the features (e.g., step(s), component(s), condition(s), parameter(s), or the like, any combination thereof) are altered during the method (e.g., during one or more or all of the steps). A dynamic method may be used to fabricate a fibrous product comprising one or more dynamic layer(s).

In various examples, the method further comprises one or more time(s) during formation of the fiber(s) one or more or all of the following: adding at least a third solvent to the fluid stock(s); altering the concentration of a conducting agent in the fluid stock(s); and altering the electric potential(s) applied to the nozzle(s), where fiber fusion, fiber stacking, or a combination thereof is altered.

In various examples, the method further comprises one or more time(s) during formation of the fiber(s) one or more or all of the following: altering the translational speed of the nozzle(s); altering the flow rate per nozzle of the fluid stock through the nozzles; and altering the fiber collection speed of the collector system, where the winding angle and/or the diameter of the fiber(s) is altered.

In various examples, the method further comprises one or more times during formation of the fiber(s) one or more or all of the following: altering the concentration of one or more of the material(s) in the fluid stock(s); altering the concentration of one or more additive(s) in the fluid stock(s); and adding or removing fluid stock(s), where the composition of the fiber(s) is altered.

In an aspect, the present disclosure provides products. In various examples, a product is made using a system and/or by method of the present disclosure. A product may be referred to, in the alterative, as a fibrous product. Non-limiting examples of products are provided herein.

A product can comprise various layer(s) of fibers. In various examples, the product comprises one or more layer(s) of fibers. A layer can comprise various fibers. In various examples, all of the layers in a product are the same. In various examples, at least one portion of a layer and/or at least one layer comprises fibers that have at least one feature (e.g., structural feature, geometrical feature, compositional feature, or the like) that is different than the fibers of the other portion(s) of the fibers in a layer or other fibers in a layer.

In various examples, a fiber is a microfiber. In various examples, the product comprises three or more layer(s) of fibers. In various examples, the fibers are arranged in a predetermined pattern; the average diameter of the fibers is from about 100 nm to about 500 microns, including all 1 nm values and ranges therebetween; and the product comprises a desired fiber fusion and/or fiber stacking. In various examples, the average diameter of the fibers is from about 200 microns to at least 500 microns.

The product can comprise various types and degrees of desired fiber fusion and/or fiber stacking. In various examples, the fibers comprise a plurality of fusion points between two intersected fibers, such that for each fusion point, a bottom surface of a first fiber is bonded to a top surface of a second fiber. In various examples, for at least one or more or all fusion points, the overlapped distance of the first fiber and the second fiber is less than about 15%, less than about 10%, less than about 5%, less than about 4%, less than about 3%, less than about 2%, or less than about 1% of the diameter of the first fiber and/or second fiber.

In various examples, the fibers comprise an average frequency of fusion points from about 5% to about 99%, including all 1% values and ranges therebetween. In various examples, the fibers comprise an average frequency of fusion points of from about 5% to about 99%, or from about 5% to about 90%, or from about 10% to about 60%, or at least about 5%, or at least about 7%, or at least about 10%, or at least about 15%, or at least about 20%, or at least about 30%, or at most about 99%, or at most about 90%, or at most about 80%, or at most about 70%, or at most about 60%, or at most about 50%, including all 1% values and ranges therebetween.

In various examples, for each axial direction of the product, the adjacent fibers in different layers are aligned one over the other and are vertically stacked or vertically staggered. In various examples, the fibers have an average stacking height of at least about 10 microns, or at least about 20 microns, or at least about 30 microns, or at least about 40 microns, or at least about 50 microns, or at least about 60 microns, or at least about 70 microns, or at least about 80 microns, or at least about 90 microns, or at least about 100 microns, or at least about 150 microns, or at least about 200 microns, or at least about 250 microns.

The product can comprise various predetermined patterns. In various examples, each layer comprises one or more group(s) of fibers optionally aligned in one or more axial direction(s) of the product within and/or between the layer(s). In various examples, the group(s) of fibers is/are uniaxially, biaxially, or multi-axially oriented within and/or between the layer(s). In various examples, each group of fibers has a substantially constant winding angle. In various examples, the winding angle is from about 1° to about 89°, including all 0.1° values and ranges therebetween. In various examples, the product comprises two groups of aligned fibers aligned in two directions with a substantially constant winding angle from about 15° to about 90°, from about 25° to about 80° or more preferably from about 35° to about 75°, including all 0.1° values and ranges therebetween, relative to the longitudinal axis of the product.

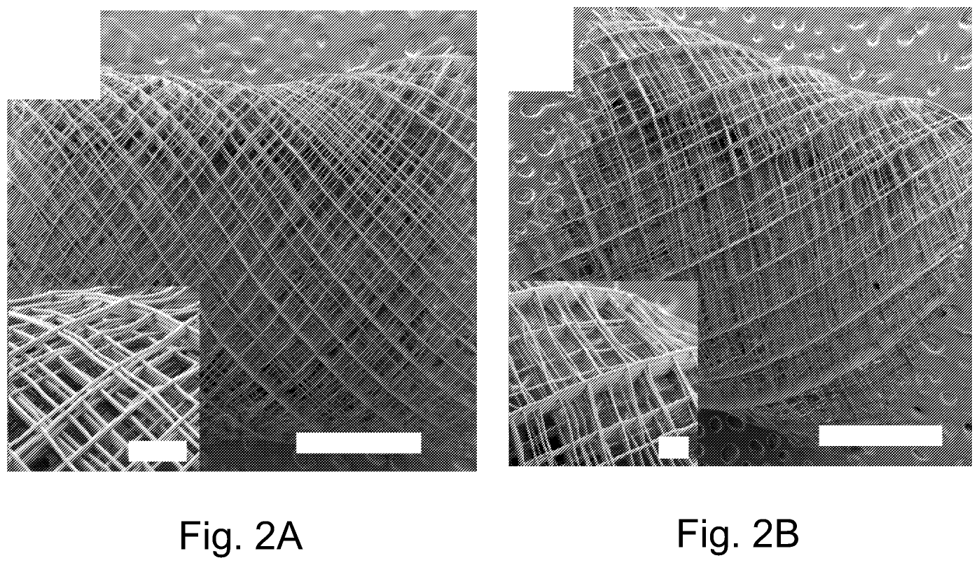

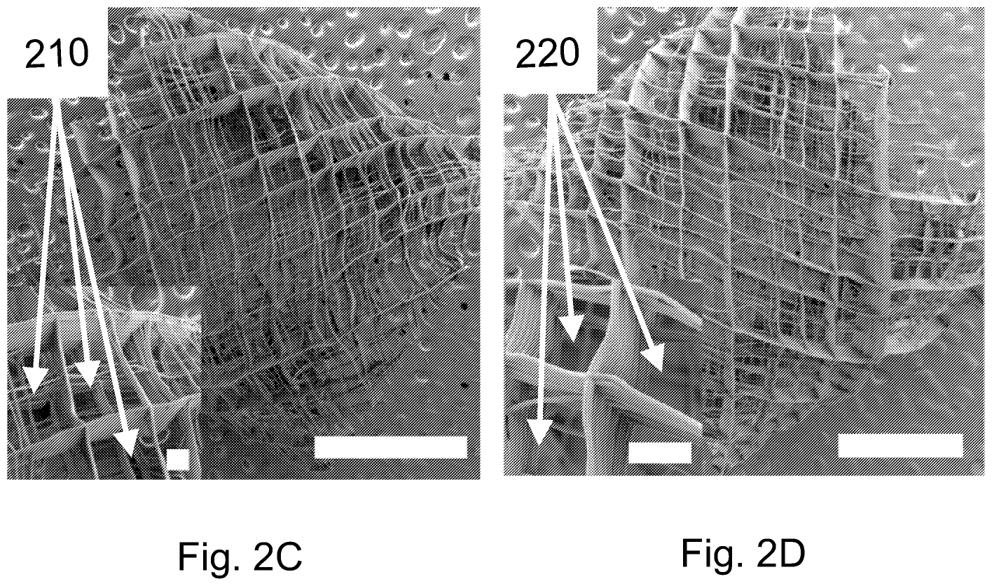

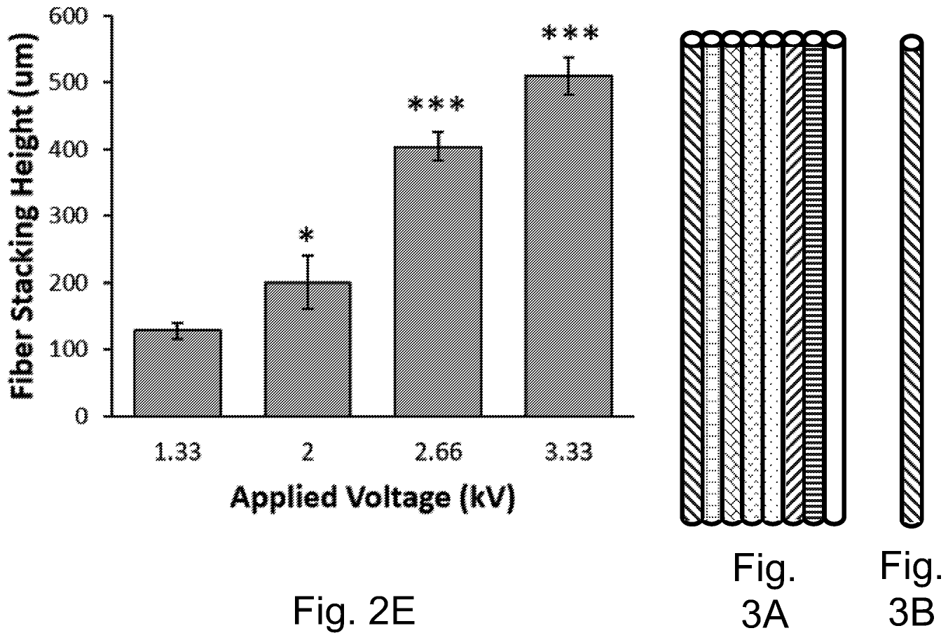

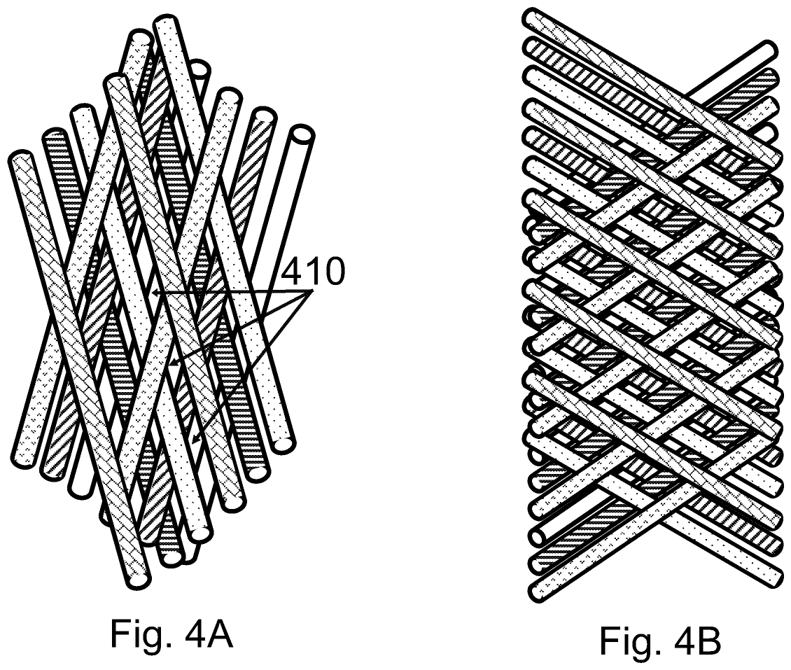

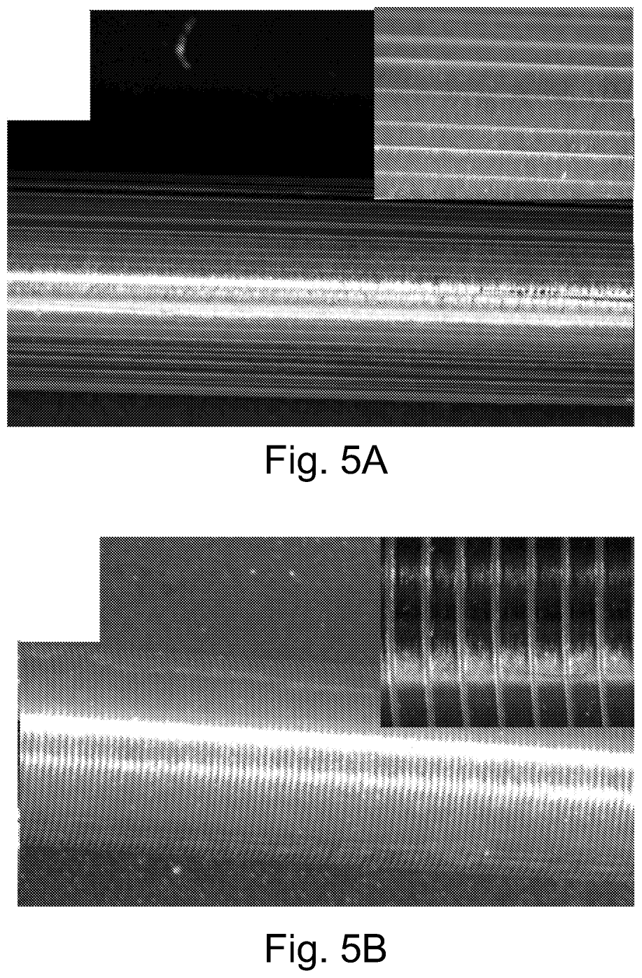

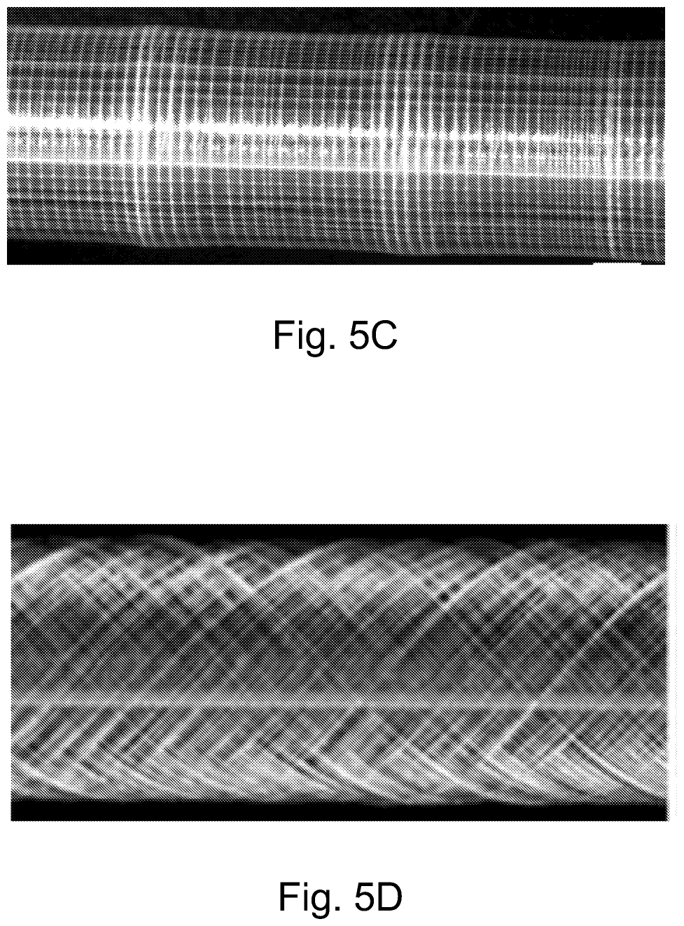

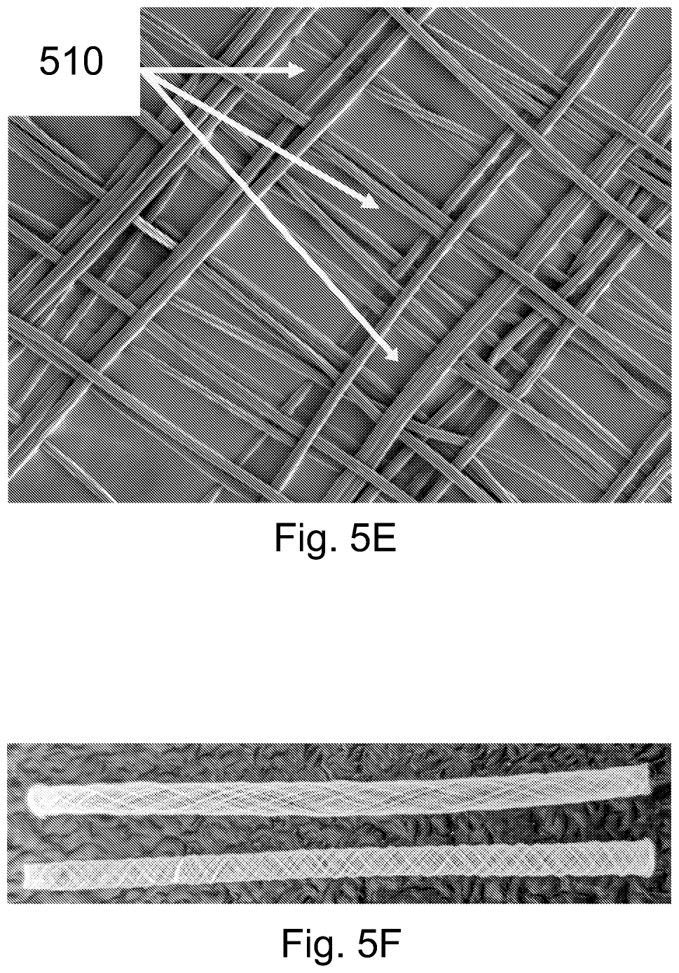

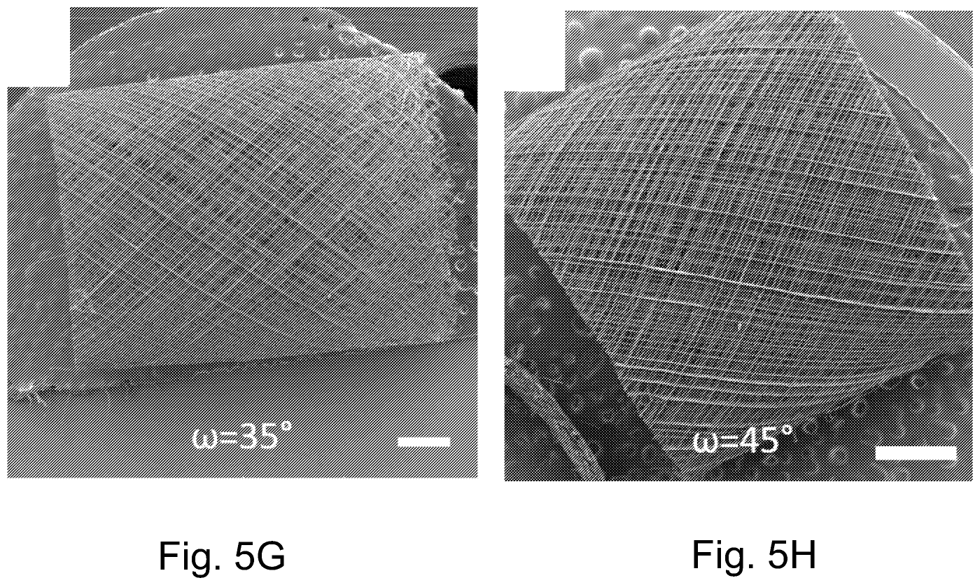

In various examples, the product comprises a plurality of pores. In various examples, the pores are defined by a plurality of fibers. In various examples, the pores are defined by a plurality of stacked fibers, a plurality of distributed fibers, or the like, or any combination thereof. Nonlimiting examples of pores defined by stacked fibers include pores 210 and 220 shown by way of example in FIGS. 2C-2D. Nonlimiting examples of pores defined by distributed fibers include pores 410 shown by way of example in FIG. 4A and pores 510 shown by way of example in FIG. 5E. For instance, a pore may be defined by a fiber of a first layer, spaced apart by a first predetermined space or pitch from the same fiber, or another fiber, on the first layer, and the fiber of a second layer, spaced apart by a second predetermined space or pitch, which may be equal to or different than the first predetermined space or pitch, with same fiber, or another fiber, on the second layer, with the fiber(s) of the first layer and the fiber(s) of the second layer being arranged at an angle relative to one another between 0° to 90°. In another example, a pore may be defined by fiber(s) of a first plurality of layers, spaced apart by a first predetermined space or pitch (which could comprise a varying space or pitch across the first plurality of layers) from the same fiber(s), or other fiber(s), comprising the first plurality of layers, and the fiber(s) of a second plurality of layers, spaced apart by a second predetermined space or pitch, which may be equal to or different than the first predetermined space or pitch (which could comprise a varying space or pitch across the second plurality of layers), with same fiber, or another fiber, on the second plurality of layers, with the fiber(s) of the first plurality of layers and the fiber(s) of the second plurality of layers being arranged at an angle relative to one another between 0° to 90°.

In some examples, one or more pores are formed, by arrangement of fiber(s) across a thickness of the product, to extend through the product (e.g., from an outer diameter to an inner diameter of a cylindrical product) or to extend only partially through a thickness of the product. In a substantially cylindrical product, for example, an axis of a pore may be described as extending from a first position (ρ₁, φ₁, z₁) in a cylindrical coordinate system for the product to a second position (ρ₂, φ₂, z₂) in the cylindrical coordinate system wherein ρ₁, φ₁ and/or z₁ may be equal to, or different than, ρ₂, φ₂, z₂. Accordingly, in some examples, pores extend radially outwardly relative to a longitudinal axis of a substantially cylindrical product, whereas in some examples pores extend both outwardly relative to a longitudinal axis of a substantially cylindrical product and along the longitudinal axis and/or circumferentially.

In various examples, the average width of the pores is at least about 1 micron. In various examples, the average width of the pores is at least about 1 microns, at least about microns, at least about 10 microns, at least about 20 microns, at least about 30 microns, at least about 40 microns, or at least about 50 microns. In various examples, the pores have a cube shape, a cuboid shape, a rhombohedron shape, a rhomboid shape, or the like.

The product can comprise fiber(s) comprising various material(s). In various examples, each fiber comprises one or more material(s) which is/are thermo-reactive at a temperature of at least 60° C. In various examples, the one or more material(s) is/are thermo-reactive at a temperature of at least 60° C., at least 100° C., at least 150° C., at least 166° C., at least 170° C., at least 180° C., at least 190° C., at least 200° C., at least 210° C., at least 220° C., at least 230° C., at least 240° C., at least 250° C., at least 260° C., at least 270° C., at least 280° C., at least 290° C., or at least 300° C.

In various examples, the one or more material(s) comprise(s) at least one polymer. In various examples, the one or more material(s) comprise at least two polymers. In various examples, the weight ratio of the two polymers is from about 1:9 to about 9:1, from about 1:5 to 5:1, from about 1:4 to about 4:1, from about 1:3 to about 3:1, from about 1:2 to about 2:1, at least about 1:99, about 1:9, about 1:8, about 1:7, about 1:6, about 1:5, about 1:4, about 1:3, about 1:2, or about 1:1, or at most about 99:1, at most about 9:1, about 8:1, about 7:1, about 6:1, about 5:1, about 4:1, about 3:1, about 2:1, or about 1:1, including all integer weight ratio values and ranges therebetween.

In various examples, the at least one polymer comprises a cured (e.g., thermally cured, electromagnetic radiation cured, accelerated particle cured, or the like, or any combination thereof) polymer, or the like, or any combination thereof. In various examples, the at least one polymer does not comprise a cured (e.g., thermally cured, electromagnetic radiation cured, accelerated particle cured, or the like, or any combination thereof) polymer, or the like, or any combination thereof.

In various examples, the at least one polymer comprises a crosslinked (e.g., thermally crosslinked, electromagnetic radiation crosslinked, accelerated particle crosslinked, or the like, or any combination thereof) polymer, hydrogel, or the like, or any combination thereof. In various examples, the at least one polymer does not comprise a crosslinked (e.g., thermally crosslinked, electromagnetic radiation crosslinked, accelerated particle crosslinked, or the like, or any combination thereof) polymer, hydrogel, or the like, or any combination thereof.

In various examples, the at least one polymer comprises at least one biocompatible polymer, at least one biodegradable polymer, or the like, or any combination thereof. In various examples, the product comprises at least two biodegradable polymers, wherein a first biodegradable polymer has a degradation rate of at least 50% of the polymer degraded in less than about one year, less than about 9 months, less than about 6 months, less than about 3 months in a physiological environment or a biological environment; and a second biodegradable polymer has a degradation rate of at most about 50% of the polymer degraded in more than about one year, more than about 1.5 years, more than about 2 years, more than about 2.5 years, or more than about 3 years in a physiological environment or a biological environment.

In various examples, the at least one polymer is thermo-reactive at a temperature of at least about 60° C. In various examples, the at least one polymer is thermo-reactive at a temperature of at least 60° C., at least 100° C., at least 150° C., at least 166° C., at least 170° C., at least 180° C., at least 190° C., at least 200° C., at least 210° C., at least 220° C., at least 230° C., at least 240° C., at least 250° C., at least 260° C., at least 270° C., at least 280° C., at least 290° C., or at least 300° C.

In various examples, the at least one polymer, is a synthetic organic polymer, a natural organic polymer, or an inorganic polymer. In various examples, the at least one polymer is chosen from a polyester, polyurethane, polyether, polyketal, polyamide, polyimide, polycarbonate, polyacrylate, polysaccharide, and any combination thereof. In various examples, the at least one polymer is chosen from polyglycolide or a polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxy valerate) (PHBV), polyethylene terephthalate (PET), polybutylene terephthalate (PBT), polyethylene glycol (PEG), polytrimethylene terephthalate (PTT), polyethylene naphthalate (PEN), poly(glycerol-sebacate) (PGS), palmitate functionalized poly(glycerol sebacate (PGSP), poly(epsilon caprolactone) (PCL), polymethyl methacrylate (PMMA), chitosan, gelatin, cellulose, polyvinyl alcohol (PVA), polyvinyl pyrrolidone (PVP), polydioxanone, derivatives thereof, and the like, and any combination thereof. In various examples, the at least one polymer is chosen from: silk, gelatin, and a polysaccharide, such as, for example, cellulose, chitin, chitosan, hyaluronic acid, dextran, and alginate, and the like, and any combination thereof. In various examples, the at least one polymer is chosen from: polysilanes, polysiloxanes, polysulfides, polysilazanes, polyphosphazenes, and the like, and any combination thereof. In various examples, at least one fiber comprises at least one first polymer and at least one second polymer, and/or where at least one first fiber comprises at least one first polymer and at least second fiber comprises at least one second polymer. In various examples, the at least one first polymer is PGSP and/or the at least one second polymer is Polyethylene terephthalate (PET).

In various examples, at least one fiber further comprises at least one additive. In various examples, the additive is uniformly distributed in at least one fiber and/or encapsulated by or attached to the polymer(s) in at least one fiber. In various examples, the at least one additive is chosen from a therapeutic agent, a dye, an indicator agent, a drug, and the like, and any combination thereof. In various examples, the additive is a therapeutic agent or a drug, configured to gradually deliver to a physiological environment after implanting the product into the physiological environment.

The product can comprise various morphological and/or structural feature(s). In various examples, the product has an inner diameter of from about 0.5 mm to about 300 mm, including all 0.01 mm values and ranges therebetween, and/or an outer diameter of from about 0.51 mm to about 300 mm, including all 0.01 mm values and ranges therebetween. In various examples, the product is a conduit, a web, a patch, a mat, a cuff, or the like. In various examples, the product comprises a shape of at least a portion of an organ, a vessel, a body part, or the like. In various examples, the product is a conduit, a web, a patch, a mat, or a cuff, comprising a shape of at least a portion of an organ, a vessel, a body part, or the like.

A product may be asymmetric. In various examples, a product is asymmetric in terms of one or more dimension(s), one or more mechanical propert(ies), fiber composition, or fiber structure (e.g., fiber fusion, fiber stacking, pore shape, pore size, or the like), or any combination thereof. In various examples, product asymmetry is in terms of fiber alignment, fiber orientation, winding angle, or the like. As an illustrative example, one or more of the aforementioned features of a product (such as, for example, a conduit or the like) is altered from an end (e.g., an orifice) to another end (e.g., another orifice). In various examples, an asymmetric product is a scaffold or graft, which may be used as an artificial trachea, artificial bronchia, an arteriovenous graft, or the like.

An arteriovenous graft may be asymmetric. In various examples, an arteriovenous graft is asymmetric in terms of orifice or end size (e.g., linear inner dimension, such as, for example, a diameter or the like), orifice or end wall thickness, or the like. In various examples, an arteriovenous graft accommodates the size “mismatch” between arteries and veins (arteries are typically smaller in diameter but with thicker wall) for both conduit diameter and wall thickness. In various example, an arteriovenous graft comprises a first end (which may be a venous end) that is wider (e.g., has a larger inner diameter) and/or is thinner (e.g., has a thinner wall thickness) than a second end (which may be an arterial end).

The product can have various medical applications, pharmaceutical applications, industrial applications, or the like, or any combination thereof. In various examples, the product can be used for thermal insulation, for gas or liquid filtration, as a membrane, as a fabric, as a composite material, or the like.

The product can be used for various medical applications. In various examples, the product is an implantable medical device, a scaffold of an artificial tissue, or the like. In various examples, a product is a sheet, tube, mesh, pseudo 3-dimensional construct, or the like.

The shape of a product may also be manipulated for specific tissue engineering applications. Exemplary shapes include, but are not limited to, particles, tubes, spheres, strands, coiled strands, films, sheets, fibers, meshes, foams, and the like, and any combination thereof. In various examples, a product has high porosity, low porosity, or a combination of different porosities. In some examples, the constructs are vascularized (micro-channeled) fibrous sheets, random meshes, aligned sheets, cylindrical tubes, or pseudo 3-dimensional constructs, such as, for example, shapes to mimic organs or the like. These structures are particularly useful for applications in soft and elastomeric tissues.

A product can be a tissue graft, a scaffold, or the like. In various examples, the solution electrowritten product is a tissue graft, scaffold (such as, for example, a tissue engineering scaffold or the like), or the like. In various examples, a tissue graft or a scaffold (e.g., prior to use, such as, for example, implantation or the like) is substantially cell-free or cell free.

In various examples, a tissue graft or scaffold is used for the replacement and/or repair of damaged native tissues. In various examples, a scaffold is used in in situ tissue engineering applications, including, but not limited to, vascular grafts, bone, intestine, liver, lung, or any tissue with sufficient progenitor/stem cells. In various examples, a tissue graft or scaffold is useful for regenerating tissues that are subject to repeated tensile, hydrostatic, or other stresses, such as, for example, lung, blood vessels, heart valve, bladder, cartilage, muscle, and the like. For example, a tissue graft or scaffold is contemplated to be implantable for tensile load bearing applications, such as, for example, tubular networks with a finite number of inlets and outlets, tubes configured to act as artery interpositional grafts, and the like.

A product may seeded with cells or implanted directly and relying on the host to serve as cell source and “bioreactor”. These structures can be implanted as artificial organs and the inlets and outlets will be connected to host tissues, vasculature, or the like. In some examples, the vasculature itself is valuable without parenchymal cells. For example, in treating ischemic diseases. The microvascular mimetics can be connected directly to a host vessel and perfuse an ischemic area of the body. In some examples, a tissue graft or scaffold, such as, for example, a vascular graft or the like, is cell-free, in which it does not include any living cells, such as, for example, smooth muscle cells, endothelial cells, or the like, or any combination thereof.

Without intending to be bound by any particular theory, it is considered that a product (e.g., a tissue graft or scaffold) can guide host tissue remodeling in many different tissues, including any tissue that has progenitor cells. For example, a biodegradable scaffolds is used to facilitate tissue regeneration in vivo by providing a structural frame for which tissue regeneration can occur. In some examples, a product allows and facilitates infiltration of host cells, including progenitor cells and the like. In some examples, a product allows and facilitates host remodeling of the biodegradable structure, so that the polymeric structure is replaced by the desirable host tissue. It is contemplated that the methods of fabrication and/or systems disclosed herein can be modified as desired by one of ordinary skill in the art to fabricate a product with the appropriate dimensions and features depending upon tissue which is to be replaced.

The various dimensions of a tissue graft or scaffold can vary according to the desired use. In principle, the dimensions will be similar to those of the host tissue in which the scaffold/graft is being used to replace. For example, in the case of a vascular graft, a vascular graft has an inner diameter which matches that of the host vessel to be replaced. However, it is contemplated the graft wall can be fabricated with a thicker or thinner wall than that which is being replaced, if desired. Typically, the wall thickness of a disclosed scaffold or vascular graft is designed to match that of the host tissue or vessel to be replaced.

In various examples, a tissue graft is a soft tissue graft or the like. In various examples, a tissue graft is a soft tissue graft (such as, for example, blood vessel grafts, muscle grafts, skin grafts, ligament grafts, internal organs (such as, for example, lungs, kidneys, hearts or the like), nervous system tissue grafts or the like), or the like. In various examples, a soft tissue graft is a vascular graft or the like. In various examples, a vascular graft is an arterial graft or the like. In various examples, an arterial graft comprises a lumen diameter of 6 mm or less.

In some examples, a disclosed vascular graft is used to form a blood vessel in vivo. For example, a disclosed vascular graft can be implanted into a subject in need of vascular graft at the desired location to form a conduit in which blood can initial flow and ultimately form a blood vessel. In various examples, the vascular graft is used as a coronary or a peripheral arterial graft, venous grafts, lymphatic vessels, or the like. In some examples, the vascular graft is used as an arteriovenous shunt for dialysis access where “maturation” of 2-3 months is common.

In various examples, a scaffold is biodegradable and/or biocompatible. In some examples, a disclosed scaffold includes PGS and/or one or more of the following polymers: polylactides (PLAs), poly(lactide-co-glycolides) (PLGAs), poly(dioxanone), polyphosphazenes, polyphosphoesters (such as, poly[1,4-bis(hydroxyethyl)terephthalate-alt-ethyloxyphosphate]; poly[1,4-bis(hydroxyethyl)terephthalate-alt-ethyloxyphosphate]-co-1,4-bis(hydroxyethyl)terephthalate-co-terephthalate; poly[(lactide-co-ethylene glycol)-co-ethyloxyphosphate]); polycaprolactone; poly(urethanes), polyglycolides (PGA) polyanhydrides, and polyorthoesters or any other similar synthetic polymers that may be developed that are biologically compatible.

The term “biologically compatible, synthetic polymers” includes copolymers and blends, and any other combinations of the forgoing either together or with other polymers generally. The use of these polymers will depend on given applications and specifications required. A more detailed discussion of these polymers and types of polymers is set forth in Brannon-Peppas, Lisa, “Polymers in Controlled Drug Delivery,” Medical Plastics and Biomaterials, November 1997, the disclosure of which with regard to biologically compatible and/or synthetic polymers is incorporated by reference.

In some examples, the scaffold or graft includes pores to facilitate cell infiltration, but pores are not necessarily required. In some examples, the pores are uniformly distributed. In some examples, the pores are non-uniformly distributed.

In some examples, a scaffold or tissue graft includes uniformly distributed pores. In some examples, a scaffold or tissue graft includes non-uniformly distributed pores. In some examples, a scaffold or tissue graft does not include any pores. In some examples, a porous scaffold or porous tissue graft includes at least 75% pore interconnectivity, such as, for example, about 80% to about 90%, about 90% to about 98%, including 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% interconnectivity.

At least a portion or all of a scaffold or tissue graft may degrade after implantation in an individual. In some examples, at least 50%, such as, for example, about 55% to about 70%, about 80% to about 90%, about 90% to about 98%, including 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.99% of a scaffold or tissue graft (e.g., a vascular graft) degrades within one year, such as, for example, within 1 to 10 months, including within 1 month, 2 months, 3 months, 4 months, months, 6 months, 7 months, 8 months, 9 months, 10 months, 11 months, or 12 months of implantation.

In some examples, the biodegradable scaffold is coated with a biocompatible and/or biodegradable material. It is contemplated that one of ordinary skill in the art can determine with but limited experimentation, which substrates are suitable for a particular application. In some examples, the inner luminal surface of the biodegradable scaffold is coated with a biocompatible and/or biodegradable material. It is contemplated that such coating may be complete or partial. In some examples, the inner luminal surface of a biodegradable scaffold is coated completely with a thromboresistant agent, such as, for example, heparin and/or other compounds known to one of skill in the art to have similar anti-coagulant properties as heparin, to prevent, inhibit or reduce clotting within the inner lumen of the vascular graft.

In some examples, the scaffold is impregnated with any of a variety of agents, such as, for example, suitable growth factors, stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), hepatocyte growth factor (HGF), stromal cell derived factor (SDF), platelet derived growth factor (PDGF), keratinocyte growth factor (KGF), skeletal growth factor (SGF), osteoblast-derived growth factor (BDGF), insulin-like growth factor (IGF), cytokine growth factor (CGF), stem cell factor (SCF), colony stimulating factor (CSF), growth differentiation factor (GDF), integrin modulating factor (IMF), calmodulin (CaM), thymidine kinase (TK), tumor necrosis factor (TNF), growth hormone (GH), bone morphogenic proteins (BMP), interferon, interleukins, cytokines, integrin, collagen, elastin, fibrillins, fibronectin, laminin, glycosaminoglycans, heparan sulfate, chondrotin sulfate (CS), hyaluronic acid (HA), vitronectin, proteoglycans, transferrin, cytotactin, tenascin, and lymphokines.

A product may comprise (or be) a hybrid structure. In various examples, a hybrid structure comprise (or is) one or more electrowritten layer)(s) and one or more layer(s) formed using a conventional method (such as, for example, an electrospinning method or the like). The hybrid structure may comprise (or may be) one or more electrowritten layer(s) independently at least partially or completely disposed on a layer formed using a conventional method. In various examples, a hybrid structure comprises (or is) alternating electrowritten layers and layers formed using conventional methods, where each electrowritten layer may be independently at least partially or completely disposed on one or two layer(s) formed using a conventional method. In these examples, the independent electrowitten layer(s) may be an exterior layer (such as, for example, a sheath or the like) or an internal layer (such as, for example, a core or the like). A hybrid structure may comprise a core and a sheath. In various examples, a core is an electrowritten layer and a sheath is a layer formed by a conventional method (such as, for example, an electrospinning method or the like) or a core is a layer formed by a conventional method or system (such as, for example, an electrospinning method or system or the like) and a sheath, which may at least partially, substantially, or completely surround the core, an electrowritten layer is a layer formed by a conventional method. In various examples, the sheath is a microfibrous sheath or a nanofibrous sheath. In various examples, a product (e.g., a tissue graft, scaffold, or the like) comprises a biodegradable core (which may be formed using a system of the present disclosure or by a method of the present disclosure). In various examples, a tissue graft or scaffold comprises a tubular core (which may be a biodegradable polyester tubular core), such as, for example, for a vascular graft or the like. In some examples, a biodegradable polyester tubular core includes PGS. In some examples, the biodegradable polyester tubular core includes PGS and one or more biodegradable substances similar to PGS, such as, for example, a polymer or an elastomer with relatively fast degradation rate. These may include derivatives of polyglycolic acid, polycarbonate, polyurethane, polyethylene glycol, poly(orthoester), or the like, or any combination thereof. It is contemplated that a disclosed product may include PGS or any biodegradable and/or biocompatible substance with similar degradation rates and elasticity of PGS. In some examples, the product scaffold further includes a sheath which surrounds the core (which may be an electrowritten biodegradable polyester tubular core). The sheath can be formed by conventional methods, such as, for example, electrospinning or the like. In some examples, the sheath is a biodegradable polyester electrospun sheath which surrounds the solution electrowritten biodegradable polyester tubular core to prevent, inhibit or reduce bleeding from such graft. In some examples, the biodegradable polyester electrospun sheath includes PCL or a PCL like substance which is capable of forming a less leaky (compared to a product without a sheath) or substantially nonleaky (or nonleaky) sheath, which may be hydrophobic, around the biodegradable polyester electrospun sheath. In some examples, the biodegradable polyester electrospun sheath includes a hemostatic material such as, for example, gelatin or the like, or any combination thereof, which is capable of inducing hemostatis shortly after implantation. In some examples, a biodegradable scaffold does not include a sheath. For example, a biodegradable scaffold includes one or more biodegradable polyesters or like substances without a sheath. In one particular example, a biodegradable scaffold includes PGS and one or more carrier polymers, such as, for example, poly(lactic acid) (PLA), polycaprolactone (PCL), poly(glycolic acid) (PGA), the copolymer poly(lactide-co-glycolide) (PLGA), or the like, or any combination thereof. In a particular example, the biodegradable scaffold includes a PGS core surrounded by an electrospun PCL sheath.

In some particular examples, a biodegradable scaffold comprising a solution electrospun biodegradable polyester core and an electrospun biodegradable polyester electrospun outer sheath surrounding the biodegradable polyester core with or without a thromboresistant agent coating the biodegradable scaffold is used to facilitate tissue regeneration in vivo by providing a structural frame for which tissue regeneration can occur.

In some examples, a disclosed scaffold/graft includes one or more natural polymers including, but are not limited to amino acids, peptides, denatured peptides such as, for example, a gelatin from denatured collagen, polypeptides, proteins, carbohydrates, lipids, nucleic acids, glycoproteins, minerals, lipoproteins, glycolipids, glycosaminoglycans, proteoglycans, and the like, and any combination thereof. In certain examples, collagen is included. In certain examples, collagen is excluded. In certain cases, non-living macromolecular structures derived from biological tissues including, but are not limited to skins, vessels, intestines, internal organs, can be used alone or in combination with synthetic polymers named above.

A vascular graft may be an arteriovenous graft. In various examples, an arteriovenous graft is used to connect a vein and an artery. In various examples, an arteriovenous graft is used for hemodialysis access. An arteriovenous graft may be symmetric or asymmetric.

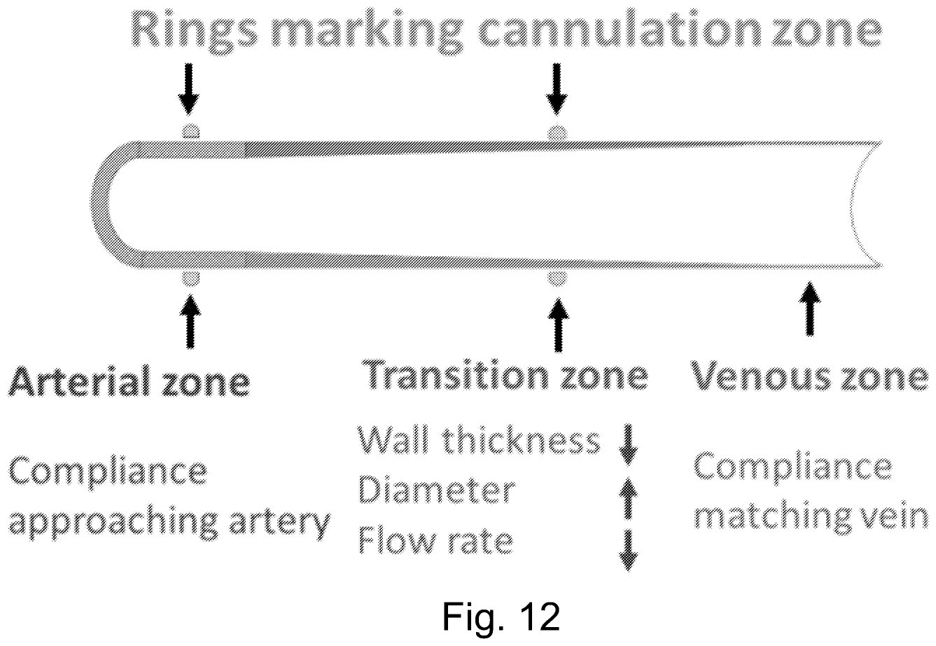

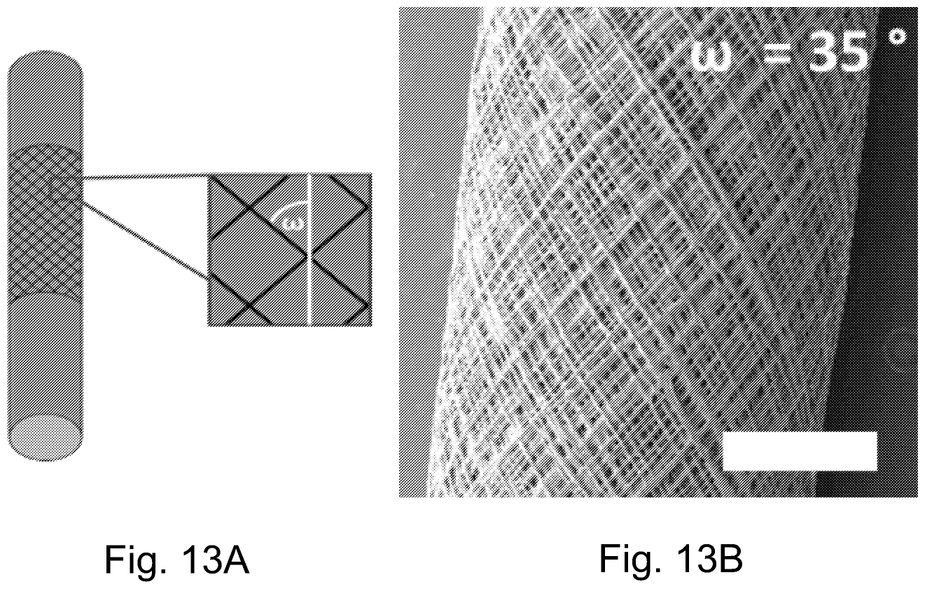

An arterio-venous (AV) graft can have various regions. Non-limiting examples of the regions of an AV graft are shown in FIG. 12. In various examples, an AV graft comprises a venous zone, a transition zone, and an arterial zone. In various examples, the AV graft comprises a first end (e.g., a venous end) or a first orifice (e.g., a venous orifice) in the venous zone. In various examples, the AV graft comprises a second end (e.g., arterial end) or a second orifice (e.g., an arterial orifice) in the arterial zone. In various examples, an AV graft comprises a transition zone between the first and second ends or orifices. In various examples, an AV graft comprises gradually decreasing diameter accompanied by increasing wall thickness moving along the transition zone from the first to the second end or orifice.

In various examples, an arteriovenous graft has an inner diameter of from about 3 to about 20 mm, including all 0.01 mm values and ranges therebetween, at an end (which may be a first end, venous end or wide end) to about 2 to about 10 mm, including all mm values and ranges therebetween, at the other end (which may a second end, opposite end, arterial end, or a narrow end) of the graft. In various examples, an arteriovenous graft includes two orifices. In various examples, an arteriovenous graft includes a first orifice (e.g., a venous orifice) and a second orifice (e.g., an arterial orifice). In various examples, the wall thickness of the first orifice (e.g., venous orifice) is from about 0.05 to about 4 mm (e.g., from about 0.05 to about 3 mm), including all 0.01 mm values and ranges therebetween, and/or the wall thickness of the second orifice (e.g., arterial orifice) is from about 0.2 to about mm, including all 0.01 mm values and ranges therebetween. The first orifice (e.g., venous orifice) may be configured to be fluidly connected with a vein of an individual. The second orifice (e.g., arterial orifice) may be configured to be fluidly connected with an artery of an individual.

In various examples, an arteriovenous graft is asymmetric in terms of orifice or end size (e.g., linear inner dimension, such as, for example, a diameter or the like), orifice or end wall thickness, or the like. In various examples, an arteriovenous graft accommodates the size “mismatch” between arteries and veins (arteries are typically smaller in diameter but with a thicker wall) for both conduit diameter and wall thickness. In various example, an arteriovenous graft comprises a first end (which may be a venous end) that is wider (e.g., has a larger inner diameter) and/or is thinner (e.g., has a thinner wall thickness) than a second end (which may be an arterial end). Without intending to be bound by any particular theory, it is considered that the difference in hardness and/or stiffness results from the first end (which may be a venous end) being wider and/or thinner than the second end (which may be an arterial end). Arteries and veins also have different composition and organization, further contributing to the difference in biomechanics. Non-limiting examples of arteriovenous grafts are described herein. In various examples, an arteriovenous graft comprises a tapered inner diameter.

In various examples, the ratio of a first end (e.g., venous end) inner diameter to a second end (e.g., arterial end) inner diameter is from about 1.5:1 to about 10:1 (e.g., from about 1.5:1 to about 8:1), including all 0.1 values and ranges therebetween, and/or the ratio of first end (e.g., venous end) wall thickness to second end wall thickness is from about 1:1.25 to about 1:100 (e.g., from about 1:4 to about 1:10), including all 0.1 values and ranges therebetween. In various examples, the second end (which may be an arterial end) of the arteriovenous graft is harder and/or stiffer than the first end (which may be a venous end).

In various examples, an inner linear dimension of the first orifice (e.g., venous orifice), which may be an inner diameter of the first orifice, is from about 10% to about 1000% larger (e.g., from about 10% to about 200% larger), including all 0.1% values and ranges therebetween) than an inner linear dimension of the second orifice (e.g., arterial orifice), which may be an inner diameter of the second orifice. In various examples, a linear dimension of the first orifice (e.g., venous orifice), which may be a wall thickness of the first orifice, is from about 5% to about 10,000% smaller (e.g., including all 0.1% values and ranges therebetween) than a linear dimension of the second orifice (e.g., arterial orifice), which may be a wall thickness of the second orifice.

An arteriovenous graft can be used in a hemodialysis method. In various examples, a hemodialysis method comprises implanting an arteriovenous graft in an individual and subsequently carrying out hemodialysis on the individual. In various examples, the implanting comprises fluidly connecting (e.g., by suturing or the like) a first orifice (e.g., venous orifice) to a vein of an individual and/or fluidly connecting (e.g., by suturing or the like) a second orifice (e.g., arterial orifice) to an artery of the individual.

An individual may be a human or non-human mammal or other animal. Non-limiting examples of non-human animals or mammals include cows, pigs, goats, mice, rats, rabbits, cats, dogs, or other agricultural mammals, pet, or service animals, and the like.

The following Statements describe various examples of methods, products and systems of the present disclosure and are not intended to be in any way limiting:

Statement 1. A solution electrowriting system comprising:
one or more nozzle(s) (e.g., one spinneret or a plurality of spinnerets);
a material supply system comprising one or more container(s)/reservoir(s) (e.g., Syringe, Pump, mixing chamber, or syringe pump) fluidically coupled to the one or more nozzles and configured to supply one or more fluid stock(s) (e.g., a solution, or at least two solutions from at least two pumps for at least one nozzle (such as, for example, mixing solutions into one nozzle, or different solutions for different nozzles);
a collector system (e.g., a collecting substrate, a collector, collector system, or mandrel, Modular Rotating Collector System) configured to collecting fiber(s) emanating/injecting from the one or more nozzle(s);
one or more power source(s) (e.g., a High Voltage Power Supply) configured to provide one or more electric potential(s) (e.g., one or more voltage) to/between the collector and (each of) the nozzle(s) (e.g., spinneret(s));
wherein the fluid stock is/comprises a solution comprising at least one solvent (e.g., a first solvent) or at least two solvents (e.g., a first solvent and a second solvent), and at least one material (e.g., at least one polymer) configured to form at least a portion of a fiber/product by the system, wherein the material is dissolvable in at least one of the solvent(s) to form a solution and/or the material is solid or reactive to be solid in less than 50° C. (e.g., a room temperature, 25° C.);
wherein the first solvent having a high volatility with a boiling point of less than 80° C. (or less than 90° C., or less than 75° C., or less than 70° C., or less than 65° C., or less than 60° C., or less than 55° C., or less than 50° C., or less than 45° C.) and;
the second solvent having a lower volatility than the first solvent; wherein the second solvent having a boiling point of at least 10° C. higher (or at least 20° C. higher, or at least 30° C. higher, or at least 40° C. higher, or at least 50° C. higher, or at least 60° C. higher, or at least ° C. higher, or at least 80° C. higher, or at least 90° C. higher, or at least 100° C. higher, or at least 110° C. higher) than the first solvent, or the second solvent having a boiling point of at least 80° C., at least 90° C., at least 100° C., at least 110° C., at least 120° C., at least 130° C., at least 140° C., or at least 150° C.
   Statement 2. The system of Statement 1, wherein the collector or collector system is positioned in a distance from the nozzle(s)/spinneret(s) of less than about 50 mm, preferably in a range from about 500 microns to 30 mm, or more preferably in a range from 1 mm to 20 mm.
   Statement 3. The system of Statement 1, wherein the electric potential is selected from a range of 100V-8 kV, preferably a range of 100V-5000V, or more preferably a range of 1000V-5000V, or at least 100 V, at least 200 V, at least 500 V, at least 1 kV, or less than less than 8 kV, less than 5 kV, less than 4 kV, less than 3 kV, or any subranges therein.
   Statement 4. The system of Statement 1, further comprising a motorized stage configured to move the nozzle(s)/spinneret(s) during the electrowriting, or wherein the nozzle(s) or spinneret(s) comprising moving nozzle(s) or spinneret(s)
   Statement 5. The system of Statement 1, wherein the collector comprising a moving collector (e.g., a rotating mandrel, or a (second) motorized stage configured to move the collector). The system can choose either moving nozzle(s) or moving collector, or the combination thereof.
   Statement 6. The system of Statement 1, wherein at least a portion of the fluid stock further comprising one or more additive(s) selected from a conductive agent, a therapeutic agent, a dye, an indicator agent, a drug, or any combination thereof.
   Statement 7. The system of Statement 1, wherein the temperature of the fluid stock (solution) is less than 80° C., less than 70° C., less than 60° C., less than 50° C., less than 40° C., less than ° C. before, during and/or after the fluid stock coming (or spinning) out of the nozzle(s).
   Statement 8. The system of Statement 1, wherein the material in the fluid stock comprising a polymer having a melting temperature of at least 60° C., at least 100° C., at least 150° C., at least 166° C., at least 170° C., at least 180° C., at least 190° C., at least 200° C., at least 210° C., at least 220° C., at least 230° C., at least 240° C., at least 250° C., at least 260° C., at least 270° C., at least 280° C., at least 290° C., or at least 300° C.
   Statement 9. The system of Statement 1, wherein the fluid stock is a solution comprising two polymers and/or at least two solvents.
   Statement 10. The system of Statement 1, wherein the fluid stock comprising a first polymer having a degradation rate of at least 50% of the polymer degraded in less than one year, less than 9 month, less than 6 month, less than 3 month in a physiological environment or a biological environment; and a second polymer having a degradation rate of at most 50% of the polymer degraded in more than one year, more than 1.5 year, more than 2 year, more than 2.5 year, or more than 3 year in a physiological environment or a biological environment.
   Statement 11. The system of Statement 5, wherein the rotating mandrel rotates with a rotation speed (VR) selected from a range of 0.5-20 cm/s or preferably 1-10 cm/s.
   Statement 12. The system of Statement 1, wherein the first solvent is 10%-100% of the total solvent, or 20%-80% of the total solvent, or preferably 25%-75% of the total solvent.
   Statement 13. The system of Statement 1, wherein the second solvent is 1%-90% of the total solvent, or 10%-80% of the total solvent, or preferably 25%-75% of the total solvent.
   Statement 14. The system of Statement 1, wherein the ratio of first solvent to the second solvent is selected from a range of 1:99-99:1, or 1:9 to 9:1, or 1:4 to 4:1, or 1:3 to 3:1, at least 1:99, 1:9, 1:8, 1:7, 1:6, 1:5, 1:4, 1:3, 1:2, or 1:1, or at most 99:1, at most 9:1, 8:1, 7:1, 6:1, 5:1, 4:1, 3:1, 2:1, or 1:1, or any combination thereof.
   Statement 15. The system of Statement 1, where the first solvent is selected from HFIP, Dichloromethane, acetone, Chloroform, Methanol, Tetrahydrofuran or any combination thereof.
   Statement 16. The system of Statement 1, where the second solvent is selected from Anisole, N,N-Dimethylformamide, Dioxane, Dimethyl Sulfoxide (DMSO) or any combination thereof.
   Statement 17. The system of Statement 1, where the fluid stock has a polymer concentration selected from a range of 5% to 50% W/V, preferably 15% to 40% W/V, or more preferably 20% to 30% W/V.
   Statement 18. The system of Statement 1, comprising a plurality of nozzles, or at least three nozzles, or an array of nozzles or two array of nozzles.
   Statement 19. The system of Statement 1, comprising a first nozzle or first array of nozzles configured to form a group of filaments uniformly aligned in a first direction, and a second nozzle or second array of nozzles configured to form a group of filaments uniformly aligned in a second direction; and optionally the first direction and the second direction forms an angle with a degree selected from a range of 15° to 90°, or a range of 25° to 80° or more preferably from 35° to 75°.
   Statement 20. A method of controlling a fiber stacking for solution electrowriting, comprising providing a system of Statement 1, increasing the ratio of the first solvent to the second solvent to increase the fiber stacking height for the same layers of stacking fibers with substantially the same diameter of fibers; and/or decreasing the ratio of the first solvent to the second solvent to decrease the fiber staking height for the same layers of stacking fibers with substantially the same diameter of fibers.
   Statement 21. A method of controlling a fiber fusion for solution electrowriting, comprising providing a system of Statement 1, increasing the ratio of the first solvent to the second solvent to decrease the fiber fusion in the intersection(s) of fibers (e.g., fiber crossing points), and/or decreasing the ratio of the first solvent to the second solvent to increase the fiber fusion in the intersection of fibers.
   Statement 22. A product made by the system of Statement 1, comprising at least three layers of (uniaxially, or biaxially, or multi-axially, oriented) fibers, wherein for each axis/orientation/direction, the adjacent fibers in different layers are vertically stacked and aligned one over another; and the adjacent fibers in the same layer are horizontally separated in a substantially constant distance (or space apart).
   Statement 23. A product made by the system of Statement 1, comprising at least three layers of (uniaxially, or biaxially, or multi-axially, oriented) fibers, wherein for each axis/orientation/direction, the adjacent fibers in different layers are vertically staggered and aligning one over another; and the adjacent fibers in the same layer are horizontally separated in a distance.
   Statement 24. A product made by the system of Statement 1, comprising two groups of aligned fibers (biaxially aligned fibers) aligned in two directions with a substantially constant winding angle, wherein the winding angle is selected from a range of 15° to 90°, a range of to 80° or more preferably from 35° to 75°.
   Statement 25. A method of making a biocompatible scaffold, comprising providing a fluid stock comprising at least one biocompatible polymer and at least two solvents, wherein a first solvent having high volatility with a boiling temperature of less than 80° C. (or less than 90° C., or less than 75° C., or less than 70° C., or less than 65° C., or less than 60° C., or less than 55° C., or less than 50° C., or less than 45° C.) and;
a second solvent having a lower volatility than the first solvent; wherein the second solvent having a boiling temperature of at least 10° C. higher (or at least 20° C. higher, or at least 30° C. higher, or at least 40° C. higher, or al least 50° C. higher, or at least 60° C. higher, or at least ° C. higher, or at least 80° C. higher, or at least 90° C. higher, or at least 100° C. higher, or at least 110° C. higher) than the first solvent, or the second solvent having a boiling temperature of at least 80° C., at least 90° C., at least 100° C., at least 110° C., at least 120° C., at least 130° C., at least 140° C., or at least 150° C.;
injecting/electrospinning/electrowriting the fluid stock (at a temperature of less than 80° C., or less than 70° C., or less than 60° C., or less than 50° C., or less than 40° C., or less than 37° C.) to make one or a plurality of fibers by using an electrospinning device or an electrowriting device (in an ambient environment) to form a predetermined structure of the biocompatible scaffold;
heating the scaffold (in a temperature selected from 50° C.-160° C., or 70° C.-150° C., or 80° C.-110° C., or at least 60° C., or at least 70° C., or at least 80° C., or at least 90° C., or at least 100° C., or at most 200° C., or at most 170° C., or at most 150° C. or at most 120° C. or any combination thereof) to remove residual solvent (e.g., second solvent).
   Statement 26. The method of making a biocompatible scaffold by using the system of Statement 1.
   Statement 27. A product/scaffold comprising at least three layers of fibers, each fiber comprising at least one polymer or at least two polymers; and optionally an additive distributed in at least one fiber or at least one polymer; where the adjacent fibers in adjacent layers are staggered or vertically aligned one over another, and the adjacent fibers in the same layer are spaced from each other, optionally wherein the additive could be uniformly distributed in at least one fiber and/or encapsulated by the polymer(s) in at least one fiber. (In some examples, the polymer(s) comprise biocompatible polymer(s) and/or biodegradable polymer(s) and/or biocompatible biodegradable polymer(s).) Statement 28. The product/scaffold of Statement 27, wherein the additive is uniformly distributed in the fiber material or the polymer (in a constant concentration), or bind/attach to at least one polymer(s); and/or the additive could be dissolved in solution, or dispersed as particles in solution of claim 6.
   Statement 29 The product/scaffold of Statement 27, wherein the additive is a therapeutic agent or a drug, configured to gradually deliver to a physiological environment after implanting the product into the physiological environment.
   Statement 30. The product/scaffold of Statement 27, wherein the product comprising at least two (biocompatible) polymers, wherein a first polymer having a degradation rate of at least 50% of the polymer degraded in less than one year, less than 9 month, less than 6 month, less than 3 month in a physiological environment or a biological environment; and a second polymer having a degradation rate of at most 50% of the polymer degraded in more than one year, more than 1.5 year, more than 2 year, more than 2.5 year, or more than 3 year in a physiological environment or a biological environment.
   Statement 31. The product/scaffold of Statement 27, wherein the product is an implantable medical device or a scaffold of an artificial tissue.
   Statement 32. The product/scaffold of Statement 27, wherein the two (biocompatible) polymers are mixed to form the same/single fiber or are injected/emanated/electrospun/electrowritten from the same nozzle.
   Statement 33. The product/scaffold of Statement 27, wherein the two (biocompatible) polymers forms separate/different fibers and/or arranged in a predetermined pattern (e.g., one fiber of a first polymer is next to another fiber of a second polymer), or injected/emanated/electrospun/electrowritten from different nozzles (e.g., a first nozzle for a first polymer and an adjacent second nozzle for a second polymer).
   Statement 34. The product/scaffold of Statement 27, wherein the at least one polymer or at least two polymers is/are selected from Polyglycolide or Polyglycolic acid (PGA), Polylactic acid (PLA), Polycaprolactone (PCL), Polyhydroxyalkanoate (PHA), Polyhydroxybutyrate (PHB), Polyethylene adipate (PEA), Polybutylene succinate (PBS), Poly (3-hydroxybutyrate-co-3-hydroxy valerate) (PHBV), Polyethylene terephthalate (PET), Polybutylene terephthalate (PBT), polyethylene glycol (PEG), Polytrimethylene terephthalate (PTT), Polyethylene naphthalate (PEN), Poly(glycerol-sebacate) (PGS), Palmitate Functionalized poly(glycerol sebacate (PGSP), and/or poly(epsilon caprolactone) (PCL), or the derivatives of these polymers.
   Statement 35. The product/scaffold of Statement 27, wherein the first polymer is selected from PGSP and/or the second polymer is selected from Polyethylene terephthalate (PET).
   Statement 36. The product/scaffold of Statement 27, made by the method of claim 30 using the system of claim 1.
   Statement 37. The product/scaffold of Statement 27, wherein the fibers are biaxially oriented and aligned in two directions.
   Statement 38. The product/scaffold of Statement 27, comprising at least three layers of biaxially oriented fibers, wherein for each axis/orientation/direction, the adjacent fibers in different layers are vertically staggered/stacked and aligned one over another; and the adjacent fibers in the same layer are horizontally separated in a distance to form a plurality of pores/holes/cavities in the scaffold.
   Statement 39. The product/scaffold of Statement 27, wherein the average size of the pores/holes/cavities is larger than a size of a cell (e.g., at least 1 microns, at least 5 microns, at least 10 microns, at least 20 microns, at least 30 microns, at least 40 microns, at least 50 microns, etc.).
   Statement 40. The product/scaffold of Statement 27, wherein the average diameter of the fibers is selected from 1 nm-20 microns, at least 5 nm, at least 10 nm, at least 20 nm, at least nm, at least 100 microns, at least 200 microns, at least 500 microns, at least 1 micron, at least 5 microns, at least 10 microns, at least 20 microns, at least 30 microns, or at least 50 microns.
   Statement 41. The product/scaffold of Statement 27, wherein the scaffold is a conduit, a web, a shape of at least a portion of an organ.
   Statement 42. The product/scaffold of Statement 27, wherein fibers in the scaffold have a plurality of fusion points between two intersected fibers at an average frequency selected from a range of 5% to 99%, or 5% to 90%, or 10% to 60%, or at least 5%, or at least 7%, or at least 10%, or at least 15%, or at least 20%, or at least 30%, or at most 99%, or at most 90%, or at most 80%, or at most 70%, or at most 60%, or at most 50%.
   Statement 43. The product/scaffold of Statement 27, wherein the fibers in the scaffold have a stacking height or thickness of at least 10 microns, or at least 20 microns, or at least 30 microns, or at least 40 microns, or at least 50 microns, or at least 60 microns, or at least 70 microns, or at least 80 microns, or at least 90 microns, or at least 100 microns, or at least 150 microns, or at least 200 microns, or at least 250 microns.
   Statement 44. The product/scaffold of Statement 27, wherein the ratio of the two polymers are selected from a range of 1:9 to 9:1, 1:5 to 5:1, 1:4 to 4:1, 1:3 to 3:1, 1:2 to 2:1, at least 1:99, 1:9, 1:8, 1:7, 1:6, 1:5, 1:4, 1:3, 1:2, or 1:1, or at most 99:1, at most 9:1, 8:1, 7:1, 6:1, 4:1, 3:1, 2:1, or 1:1, or any combination thereof. Statement 45. The product/scaffold of Statement 27, further comprising a water-soluble sacrificial layer on the bottom surface of the product/scaffold configured to be dissolvable in water to facilitate scaffold removal from the collector.
   Statement 46. The product/scaffold of Statement 27, wherein the sacrificial layer comprises a sodium hyaluronate (HA).
   Statement 47. The method of Statement 26, wherein a rotation speed of the rotating mandrel (VR) is selected from a range of 0.5-20 cm/s or preferably 1-10 cm/s, a speed of the motorized stage speeds is selected from a range of 0.5-20 cm/s or preferably 1-15 cm/s.
   Statement 48. The system of Statement 1, wherein the mandrel having a diameter selected from a range of 0.5 mm to 30 mm.
   Statement 49. The product/scaffold of Statement 27, wherein fibers in the scaffold have a plurality of fusion points between two intersected fibers such that for each fusion point (or late least one fusion point) a bottom surface of a first fiber sticks to a top surface of a second fiber with an overlapped distance of less than 10% (or less than 15%, less than 5%, less than 4%, less than 3%, less than 2%, or less than 1%) of the diameter of the first fiber and/or second fiber.
   Statement 50. The product/scaffold of Statement 27, wherein the pores/holes/cavities having a shape selected from cube or cuboid.
   Statement 51. The system of Statement 6, wherein the conductive agent is selected from a salt, or a (biocompatible) conductive polymer, or a material configured to increase the electrical conductivity of the solution.

The steps of the methods described in the various examples disclosed herein are sufficient to carry out a method of the present disclosure. Thus, in various examples, a method consists essentially of a combination of the steps of the methods disclosed herein. In various other examples, a method consists of such steps.

The following examples are presented to illustrate the present disclosure. They are not intended to be limiting in any matter.

### Example 1

The following is an example of solution electrowriting systems of the present disclosure, methods of using said solution electrowriting systems, and solution electrowritten products produced using said systems and/or said methods.

Described is a systematic exploration of tubular conduit fabrication and the various solution properties and fabrication parameters that can be altered to tune scaffold physical and mechanical properties. The methods can be easily implemented with equipment for conventional electro-spinning, and offers versatility, control, and customization that is unprecedented in the solution electro-spinning literature.

New methods to fabricate fibrous conduits that can be tailored to myriad biomedical applications are described. Solution electrowriting unlocks unprecedented control over electrospun microfiber patterning with low cost and skill barriers for entry. With simple modifications to conventional electro-spinning techniques and changes to fabrication parameters, tubular conduits can be fabricated with high degrees of customization. Presented is a systematic exploration of methods to controllably alter conduit dimensions, as well as fiber fusion, patterning, and stacking to create conduits with properties suited for a desired application. These techniques represent a path forward for electro-spinning practitioners to negate the inherent instability of solution electro-spinning and gain more control over the process. This additive manufacturing technique holds much process in all realms of tissue engineering of tubular structures, which include but are not limited to blood vessel, peripheral nerve, trachea, and intestine.

The present disclosure addresses drawbacks of prior electrowriting techniques with a solution electrowriting system capable of fabricating fibrous conduits with precision and patterning capabilities comparable to its solution or melt counterparts. In doing so, presented herein is a systematic exploration of tubular conduit fabrication and the various solution properties and fabrication parameters that can be altered to tune scaffold physical and mechanical properties. The result is a path forward for reproducible, affordable, and easily customizable tubular conduit fabrication that rivals the precision of melt electrowriting with the added benefits of a solution-based approach to electro-spinning. In all prior iterations of electrowriting capable of fiber patterning, fabrication was performed using a modified a 3D-printer. This approach is appealing as it provides a ready-made platform with programmable translation in x, y, and z axes. Appreciating that this served a significant barrier to entry for potential electrowriting practitioners without 3D printing access and/or experience, disclosed herein is an example of a modified conventional electro-spinning device for solution electrowriting.

The solution electrowriting systems and methods described herein enable control of fiber placements in addition to fiber diameter and spacing in the electrospun products. Presented herein is solution electrowriting as a method to produce fibrous conduits from various polymers with tunable dimensions, fiber patterning and scaffold porosity. The technique can be easily implemented with equipment for conventional electro-spinning, and offers versatility, control, and customization that is unprecedented in the solution electro-spinning literature.

This disclosure describes new methods to fabricate fibrous conduits that can be tailored to myriad biomedical applications. Solution electrowriting unlocks unprecedented control over electrospun microfiber patterning with low cost and skill barriers for entry. To date, no research has attempted to produce tubular conduits via solution electrowriting. With simple modifications to conventional electro-spinning techniques and changes to fabrication parameters, tubular conduits can be fabricated with high degrees of customization. Presented herein is a systematic exploration of methods to controllably alter conduit dimensions, as well as fiber fusion, patterning, and stacking to create conduits with properties suited for a desired application. These techniques represent a path forward for electro-spinning practitioners to negate the inherent instability of solution electro-spinning and gain more control over the process. This additive manufacturing technique holds much process in all realms of tissue engineering of tubular structures, which include but are not limited to blood vessel, peripheral nerve, trachea, and intestine.

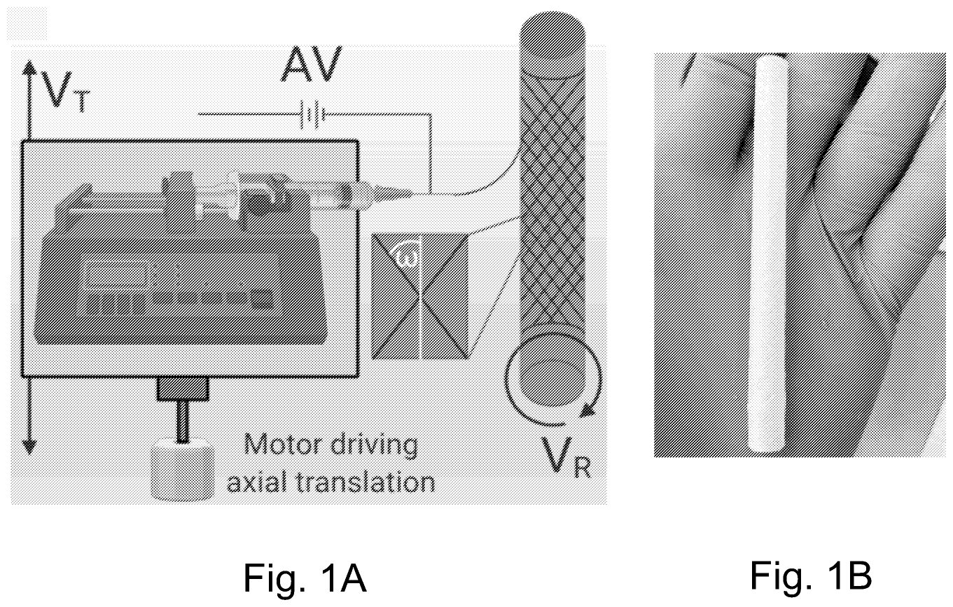

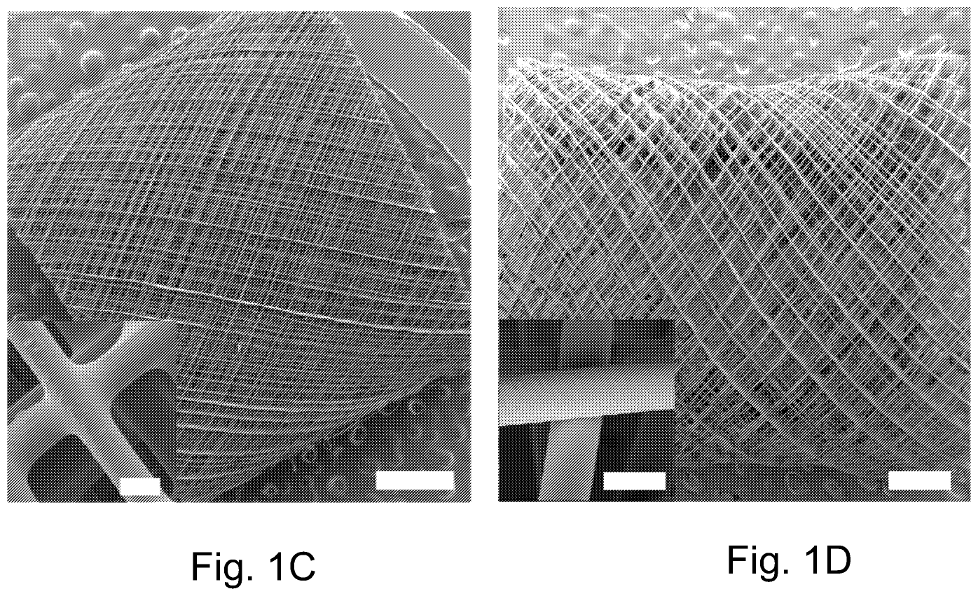

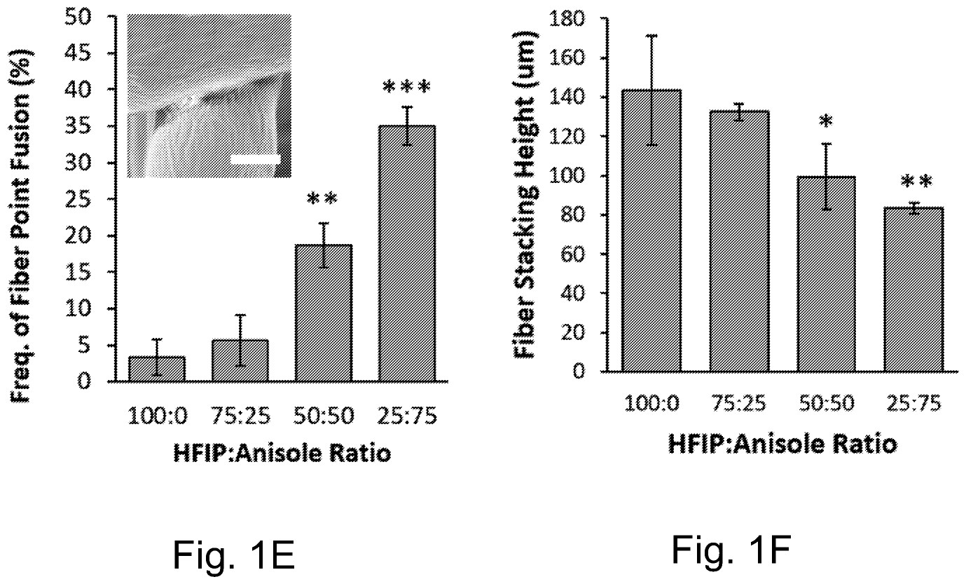

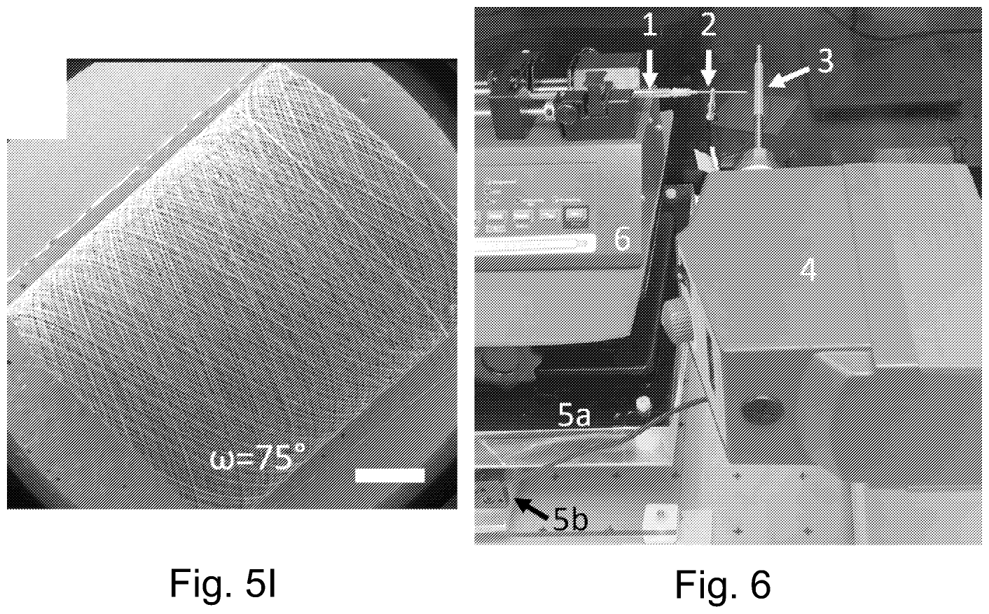

Widely available parts were used to build the presently disclosed solution electrowriting device: syringe pump, high voltage power supply, motor-driven rotating mandrel, and a motorized stage to enable programmed uniaxial translation of the spinneret (FIG. 1A and FIG. 6). This approach simultaneously bypasses the need for a 3D printer and enables easy use of a rotating fiber collection mandrel. For labs and users with electro-spinning but not 3D printing experience, this approach appreciably lowers the cost and skill barriers for entry. By translating the spinneret of a conventional electro-spinner, and finetuning electro-spinning parameters, which include solution flow rate (V), mandrel rotation speed (VR), spinneret translation speed (VT), and applied voltage, solution electrowriting is attainable for all practitioners of conventional electro-spinning.

In comparison to melt, solution electrowriting expands material selection beyond polymers with low melting points. As proof of this benefit, solution electrowriting was performed using 1,1,1,3,3,3-Hexafluoroisopropanol (HFIP) solvent with a blend of two polymers that are incompatible with melt electrowriting, as well as a polymer frequently used in melt electrowriting for comparison (polycaprolactone (PCL)). The polymer blend consisted of in-house synthesized palmitate-functionalized poly(glycerol sebacate) (PGSP), which thermally crosslinks at temperatures above 80° C., and poly(ethylene terephthalate) (PET), which has a high melting point of 260° C.

In contrast, near-field melt electro-spinning onto a rotating electro-spinning mandrel requires an expensive syringe heater to maintain the polymer in the melt phase during fabrication. Thus, melt electro-spinning is not an option when electro-spinning polymers with high melting points. For example, a polymer with a high melting point (T_{M}) that has been used in melt electrowriting to date is polypropylene (PP, T_{M}=165° C.). PP melt electrowriting required syringe heating to approximately 215° C., 50° C. higher than T_{M}. This suggests that polymers with higher T_{M} would be very difficult to process via near-field melt electro-spinning. A solution-based approach avoids this limitation.

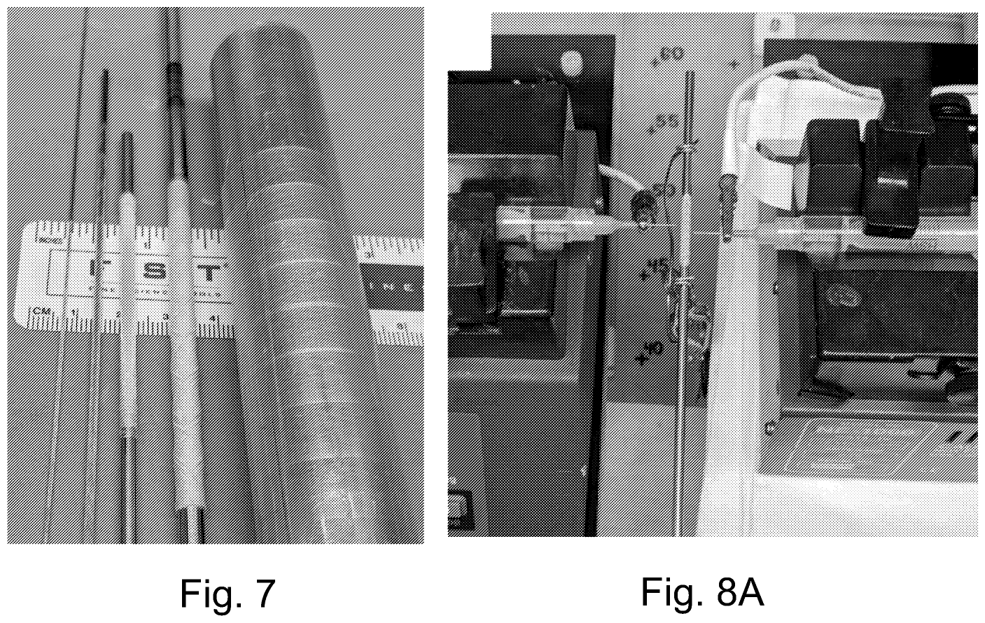

The flexibility afforded by using a modified conventional electro-spinner enables fabrication of solution electrowritten conduits of largely varying sizes. The PGSP/PET conduit in FIG. 1B has an inner diameter of 8 mm. Conduits were successfully fabricated with inner diameters ranging from 0.64 mm to 25.6 mm using mandrels of varying diameters (FIG. 7).

Conduit length is also tunable with this technique and is only limited by the range of the motorized stage translating the spinneret. Conduit thickness, fiber fusion, fiber stacking, and winding angle are also tunable with this technique and can be controlled by varying flow rate, applied voltage, spinneret translation speed, and/or mandrel rotation speed along the length of the conduit and/or between translations of the spinnerets along the length of the conduit.

The two conduits in FIG. 1C-D, made of PGSP/PET and PCL, respectively, were distinct in a few notable ways. The PGSP/PET conduit was stiffer and more resilient to the touch after thermal curing at 100° C. (PCL was not cured). Upon further inspection via scanning electron microscopy (SEM) (FIG. 1C-D insets), the two conduits showed a similar fibrous tubular structure. PGSP/PET fibers, however, fused at crossing points during thermal curing. The improved resilience and toughness resulting from fiber point fusion may be desirable in certain applications. Thus, whether point fusion could be induced during solution electrowriting to alter and improve scaffold mechanical properties was explored.

It was hypothesized that incorporating a low volatility co-solvent (e.g., boiling point>100° C.) into the electro-spinning solution would cause fibers to remain ‘wet’ with solvent as they landed on to previously deposited fibers, enabling the fibers to fuse. A parallel can be drawn from this approach to melt electrowriting where fusion at fiber cross points was observed when fibers were not allowed sufficient cooling time prior to deposition. Anisole was selected as a co-solvent due to its high boiling point (154° C.) and miscibility with HFIP. To test this hypothesis, PCL electro-spinning solutions were created with constant polymer concentration (25% m/V) in solution but varying amounts of anisole (ranging from 0% to 75% V_{Anisole}/V_{Total} with balance HFIP). After spinning these four solutions with identical fabrication parameters (Table 1) the percentage of fiber cross points exhibiting fusion increased from 3.3±2.5% to 35.0±2.6% as anisole content increased from 0% to 75% (FIG. 1E). These data supported the hypothesis that incorporating a low volatility solvent is a viable method to induce fiber point fusion during solution electrowriting.

TABLE 1

| | | | Flow | | Mandrel | Stage | | |
|---|---|---|---|---|---|---|---|---|
| | | | Rate per | Applied | Rotational | Translation | Scaffold | Collection |
| | | | Spinneret | Voltage | Speed | Speed | Length | Duration |
| Polymer | FIG. | Solvent | (μL/hr) | (kV) | (cm/s) | (cm/s) | (cm) | (min) |
| PGSP/PET | 1B | HFIP | 18.33 | 1.33 | 1.75 | 2 | 17 | 120 |
| PGSP/PET | 1C | HFIP | 18.33 | 1.33 | 1.75 | 2 | 17 | 120 |
| PCL | 1D | HFIP | 5 | 1.33 | 2.1 | 1.66 | 5.33 | 40 |
| PCL | 1E-F | HFIP/Anisole | 5 | 1.33 | 2.1 | 2 | 3.33 | 60 |
| PCL | 2A-E | HFIP | 5 | 1.33- | 1.75 | 1.66 | 5.33 | 40 |
| | | | | 3.33 | | | | |
| PCL | N/A | DCM of | 3 | 1.33 | 1.75 | 2 | 5.33 | 30 |
| | | DCM/Anisole | | | | | | |
| PCL | 5A | HFIP | 3 | 1.2 | * | 2 | 16.6 | 10 |
| PCL | 5B | HFIP | 3 | 1.2 | 1.75 | 0.02 | 16.6 | 10 |
| PGSP/PET | 5G | HFIP | 16.6 | 1.2 | 3.25 | 2 | 16.6 | 360 |
| PGSP/PET | 5H | HFIP | 18.3 | 1.2 | 3.5 | 2 | 16.6 | 360 |
| PGSP/PET | 5I | HFIP | 20.3 | 1.2 | 9.7 | 2 | 16.6 | 360 |

*Mandrel was rotated 1 step (~5°) after each spinneret translation to space fibers.

Table 1 shows other solvents that were tested for solution electrowriting. The most volatile solvent used was dichloromethane (DCM). Solution electrowriting was very difficult with this solvent and necessitated the addition of other, less volatile, solvents to enable stable fiber collection. Introducing as little as 25% (V/V) of anisole into the solution improved PCL fiber deposition stability. Other suitable high boiling point solvents (e.g., boiling point>100° C.) include N,N-dimethylformamide and dioxane, which have been successfully used to improve solution electrowriting when using solvents with low boiling points (boiling point≤50° C.). Solvents with intermediate boiling points (e.g., 50° C.<boiling point≤100° C.), such as, for example, chloroform, HFIP, THF, and TFE can be used for stable solution electrowriting with or without the addition of less volatile solvents. Table 2 shows the boiling points of common solvents suitable for use to control the volatility of the electrowriting solution.

TABLE 2

| Solvent | Boiling Point (° C.) |
|---|---|
| Diethyl Ether | 35 |
| Dichloromethane (DCM) | 40 |
| Acetone | 56 |
| 1,1,1,3,3,3-Hexafluoro-2-propanol (HFIP)* | 58 |
| Chloroform | 61 |
| Methanol | 65 |
| Tetrahydrofuran (THF) | 66 |
| Trifluoroethanol (TFE) | 77 |
| Ethanol | 78 |
| Water | 100 |
| Dioxane | 101 |
| Toluene | 111 |
| Pyridine | 115 |
| N,N-Dimethylformamide (DMF) | 152 |
| Anisole | 154 |
| Dimethyl sulfonamide (DMSO) | 189 |

Values from: www.depts.washington.edu > dielectric_chart[1]; www.stenutz.eu/chem/solv6.php?name=hexafluoroisopropanol*

A similar observation when incorporating increasing amounts of low volatility N,N-dimethylformamide (DMF) into an electro-spinning solution containing polyurethane (PU) and tetrahydrofuran (THF) has been reported. That study used conventional electro-spinning practices, and the researchers observed that larger amounts of DMF led to increased amounts of fusion between fibers. The increased fusion between PU fibers increased scaffold stiffness. In another study, a similar effect was observed during melt electrowriting of a tubular PCL scaffold. When melt electrowritten PCL fibers did not have sufficient time to cool prior to deposition they fused with already deposited fibers. When fibers were aligned closer to the angle of tension during mechanical testing, the increased stiffness resulting from fiber fusion was over-shadowed by fiber mechanical properties. The ability to controllably induce fiber point fusion during solution electrowriting, and the interplay between fiber fusion and fiber mechanical properties can allow for multiple avenues to modify conduit mechanical properties during future studies.

While using anisole to induce fiber point fusion, there was a clear decrease in the conduit wall thickness as anisole content was increased (FIG. 1F). Evidently, this occurred through a decrease in the degree of fiber stacking, i.e. fibers were better distributed over the length of the conduit, rather than stacked on top of each other. It was hypothesized that the presence of anisole decreased fiber stacking by decreasing solution polarity and the resulting charge retention in deposited fibers (decreasing solution polarity decreased the charge carrying potential of the solution which decreased the charge differential between the already-deposited fiber and the fiber emanating from the spinneret). Fibers electrospun from more polar solvents can retain electrical charge better than those spun from nonpolar solvents. These fibers then more readily take on the charge of the electrically grounded mandrel and can then attract the negatively charged fibers emanating from the spinneret. Thus, with increasing amounts of non-polar anisole, stacking was negated. To test this hypothesis, solutions containing 25% PCL (m/V) in pure HFIP were electrospun at different AV. According to this hypothesis, charge differential between deposited fibers and fibers emanating from the spinneret is the driving force for fiber attraction/stacking. Thus, it was expected that an increased applied voltage would increase the severity of fiber stacking by increasing the magnitude of the charge differential (FIGS. 2A-2E). As applied voltage increased incrementally from 1.33 kV to 3.33 kV, maximum fiber stack height increased 4-fold from 123.3±10.4 μm to 496.6±7.6 μm. Although not shown, solution electrowriting has been conducted with very low AVs (e.g., as low as 0.1 kV), so it is reasonable to believe that this effect may extend to voltages outside of the range in FIGS. 2A-2E. Electrospun scaffolds have been collected over an AV range of from about 0.1 kV to about 5 kV.

Table 3 shows common solvents in order of increasing polarity as measured by their increasing dipole moments and increasing dielectric constants.

TABLE 3

| | Dipole | Dielectric |
|---|---|---|
| SOLVENT | Moment | Constant |
| Toluene | 0.31 | 2.38 |
| Dioxane | 0.45 | 2.25 |
| Diethyl Ether | 1.15 | 4.33 |
| Chloroform | 1.15 | 4.81 |
| Anisole | 1.38 | 4.33 |
| Dichloromethane (DCM) | 1.55 | 8.93 |
| Tetrahydrofuran (THF) | 1.75 | 7.58 |
| Pyridine | 2.37 | 12.40 |
| Trifluoroethanol (TFE) | 2.52 | 8.55 |
| Acetone | 2.69 | 20.70 |
| Ethanol | 1.66 | 24.55 |
| Methanol | 2.87 | 32.70 |
| N,N-Dimethylformamide (DMF) | 3.86 | 36.71 |
| Dimethyl sulfonamide (DMSO) | 4.10 | 46.68 |
| 1,1,1,3,3,3-Hexafluoro-2-propanol (HFIP)* | 2.05 | — |
| Water | 1.87 | 80.1 |

Values from: www.depts.washington.edu > dielectric_chart[1]; www.stenutz.eu/chem/solv6.php?name=hexafluoroisopropanol*

The solvents in Table 3 are suitable in solution electrowriting alone or in mixtures to control the polarity of the electrowriting solution. The option exists to increase fiber stacking during electrowriting by using a polar solvent or mixing polar solvents to create a multi-solvent system. Stacked fibers similar to those in FIG. 2D, for example, could be linked to electrowriting polymers dissolved in a polar solvent such as, for example, pure HFIP or a solvent mixture containing polar components such as, for example, N,N-dimethylformamide, dimethyl sulfoxide, methanol, to name a few. The option exists to reduce fiber stacking during electrowriting by using a non-polar solvent, mixing non-polar solvents to create a multi-solvent system, or by diluting more polar solvents with less polar solvents to create a multi-solvent system. Distributed fibers similar to those in FIG. 2A can be linked to electrowriting polymers dissolved in a non-polar solvent, or a solvent mixture containing non-polar components such as, for example, chloroform, dichloromethane, toluene, and diethyl ether to name a few.

Many prior studies report fiber stacking during near-field electro-spinning and melt electrowriting. Melt electrowriting controls fiber stacking by directly writing fibers on top of each other. In solution electrowriting, stacking is controlled by the electrostatic attraction between already-deposited fibers, and fibers emanating from the spinneret. Prior examples of ‘controlled’ fiber stacking have been attributed to the precision of the modified 3D printer used for fabrication. The data of the present disclosure suggests that fiber stacking is not assured by precise fiber placement during solution electrowriting, but can be controlled by fabrication parameters.

Inclusion of salt was also explored as an approach to aid fabrication of patterned fibrous conduits. Altering solution conductivity was expected to increase the charge differential between fibers emanating from the spinneret and fibers already collected on the mandrel. In various examples, NaCl was added into a PEO/Water solution during near-field electro-spinning and the conductivity increase resulting from salt inclusion is expected to aid in fiber stack formation. To further demonstrate feasibility of this approach when fabricating patterned fibrous conduits via solution electrowriting, the salt LiBr (30 mM) was added into a PGSP/PET solution containing the solvents chloroform and N,N-dimethylformamide and solution electrowriting was successfully conducted to fabricate a patterned conduit. It is reasonable to assume that this approach can be used with various other salts depending on their solubility in the polymer solution of interest.

Accordingly, fiber stacking (FIGS. 3A-3B) can be induced by increasing the attraction between the fiber jet and previously deposited fibers with the inclusion of more polar solvents or increased applied voltage during fabrication or increased solution conductivity, and fiber distribution (FIGS. 4A-4B) can be induced by decreasing this attraction with the inclusion of less polar solvents or decreased applied voltage during fabrication or decreased solution conductivity. Different degrees of fiber stacking (or fiber distribution) can be achieved by altering the attraction (charge differential) between fibers already deposited on the collection mandrel and fibers emanating from the spinneret.

Carefully inducing fiber stacking in tubular conduits can be a useful tool to control transmural cell migration. One could imagine that transmural cell migration would be hindered in FIG. 2A compared to the conduit in FIG. 2D, which is increasingly anisotropic. Prior attempts to modify electrospun scaffolds porosity have taken many different approaches. The most common way to alter fibrous scaffold porosity and pore size is to change fiber diameter. Multiple studies have shown that increasing fiber diameter consequently increases scaffold pore size. Other approaches typically employ additives such as, for example, a sacrificial element that becomes embedded in the fiber mesh and then is solubilized and removed post-fabrication. Voorneveld et al. simultaneously electrospun PU and poly(ethylene oxide) (PEO) fibers. After solubilizing and removing PEO, scaffold porosity increased approximately 20%. Additional post-fabrication procedures to increase scaffold porosity have also been explored and include ultrasonication of fiber meshes, or even laser ablation of discreet locations of the scaffold. The ability to tune porosity by controlling fiber stacking during fabrication without altering fiber diameter or including additional steps during fabrication is unique and easy to implement. The added benefit of selectively inducing fiber point fusion during the process adds a layer of sophistication and customization not previously seen in the electro-spinning literature.

The fiber patterning in a tubular conduit fabricated via solution electrowriting is easily tunable by changing electro-spinning parameters. FIGS. 5A-5C show scaffolds with fibers aligned in the axial direction (FIG. 5A), the circumferential (FIG. 5B) direction, as well as a ‘cage’-design tubular scaffold containing alternating layers of circumferentially and axially aligned fibers (FIG. 5C). Fibers aligned at these extremes, as well as at interim angles, are easily achievable by adjusting VR and VT. The motorized stage was used to move the electro-spinning spinneret as fibers were deposited on the rotating mandrel, allowing the creation of helical scaffolds. Motorized stage speeds ranging from about 1 mm/sec to about 12 cm/sec were used. Optical images (FIGS. 5D, 5F) and SEM images (FIG. 5E) show scaffolds with fibers deposited with a helical orientation, including at different angles (FIG. 5F). SEM images in FIGS. 5G-5I show PGSP/PET scaffolds with fiber winding angles (w) of 45°, and 75°, respectively. These winding angles were achieved by changing mandrel rotation speed, while increasing flow rate to maintain uniform fiber diameter. The same changes can be achieved by changing spinneret translation speed instead of mandrel rotation speed. The importance of this feature stands out in applications such as, for example, a synthetic vascular graft design. Solution electrowriting can therefore enable biomimetic synthetic vascular graft fabrication tailored to specific physiological locations. Alternative solvent electro-spinning techniques, in contrast, do not allow for fine control of fiber orientation in the axial and circumferential directions as this technology does.

During solution electrowriting, as discussed thus far, a flaccid fiber is wound around a rotating mandrel to create a tightly woven fibrous conduit. If the cylindrical mandrel is replaced with another shape, perhaps 3D printed to match an irregular biological structure, it is possible to deposit fibers over the contour of this irregular shape in a similar manner as has been discussed. Since the fiber is flaccid and can be ‘sticky’ when still wet with solvent, all that would be required to achieve this is to have programmable spinneret translation over and/or around the irregular shape.

It should also be noted that the tubular conduit can be sliced to create fibrous mat of various shape: a straight cut for a rectangular mat, or a spiral cut to obtain a parallelogram shape. The curvature of the mat can be controlled by the mandrel diameter. For example, an effectively flat mat can be obtained by using large diameter mandrels.

Although the promise and potential of solution electrowriting as a biomaterial fabrication technique is clear, limitations and obstacles do exist. Similarly to melt electrowriting, depending on the size, thickness, geometry, and polymer composition of a solution electrowritten conduit, fabrication times can take many hours. For example, a PCL conduit with 6 mm inner diameter, 5 cm length, and 300 μm wall thickness can take as long as 6 hours to fabricate when using parameters similar to those reported in Table 1. Typically, this would not be a problem in a laboratory setting with a fabrication technique such as, for example, 3D printing that is automated and not prone to errors. This becomes a problem when scaling up fabrication, and when the fabrication process is prone to instability.

As previously described, in early iterations of solution electrowriting, the organic solvent used for polymer dissolution was found to largely dictate the stability of the process. In initial attempts, a solution containing 25% PCL in dichloromethane (DCM) was used. The fiber jet was found to only be capable of stable continuous fiber deposition for seconds at a time. After 5-10 seconds the fiber would dry at the tip of the spinneret. It was clear that this was a consequence of the combination of low solution flow rate and high DCM volatility. To explore this systematically and improve fiber deposition stability, anisole was incorporated into the PCL/DCM solution to determine how the addition of a low volatility co-solvent affected process stability.

Three different solutions containing 25% (m/V) PCL in dichloromethane (DCM) (100%), DCM:anisole (75:25 V/V), or DCM:anisole (50:50 V/V) were electrospun triplicate (n=3) for 30 minutes using identical processing parameters (Table 1). Using pure DCM as a solvent, stable fiber deposition was not attainable. With 75:25 DCM:anisole, stable fiber deposition was possible.

As previously described, stable fiber deposition was possible with 75:25 DCM:anisole. It was found, however, that the fiber would break once every 225±60 seconds, requiring the user to clean the spinneret before continuing. With 50:50 DCM:anisole, fiber deposition was reproducibly stable with only one fiber breakage in 90 total minutes of fabrication. The decreased volatility of the solvent mixture resulting from anisole inclusion reproducibly improved the stability of PCL fiber deposition compared to more volatile DCM. This finding was bolstered by the findings during HFIP electrowriting as previously described. PCL/HFIP solution electrowriting is remarkably stable, commonly requiring no intervention over the course of many hours of fabrication. These findings are of paramount importance for prospective solution electrowriting practitioners. Although HFIP was a great solvent candidate for conduit fabrication in the present disclosure, other polymers may require the use of different solvents. If solution electrowriting stability is difficult to achieve in those instances, the inclusion of co-solvents with lower volatility is expected to improve stability and throughput.

While the present disclosure improved the understanding of the effects of solvent volatility on solution electrowriting stability, and conduit physical properties, the tubular conduits required many hours to fabricate. To mitigate this, additional spinnerets were added into the setup. With melt electrowriting this may not be an option to increase fabrication throughput because melt electrowriting is conducted with a more sophisticated/specialized device that might not allow this modification.

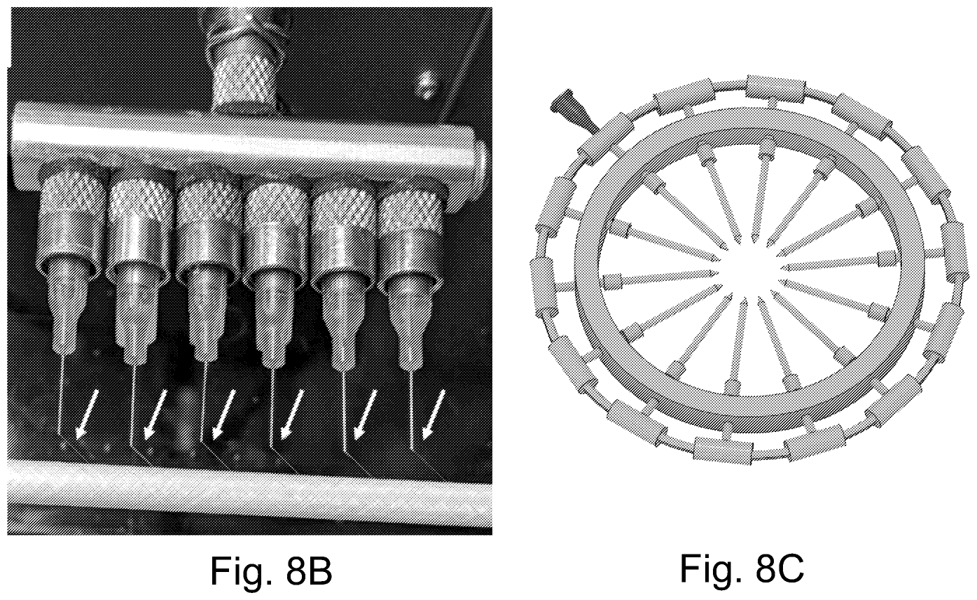

First, a second spinneret was added to the setup on the opposite side of the collection mandrel from the first spinneret (FIG. 8A). This enabled simultaneous deposition of two fibers, increasing fabrication rate by a factor of two. Although this approach does not decrease fabrication rates drastically, it allows the user to simultaneously spin two different polymer solutions to yield a hybrid conduit consisting of two interwoven, distinct fiber types.

To further decrease fabrication times, a custom-built 6-needle spinneret was introduced (FIG. 8B). This approach coupled with proper solvent selection to maximize fiber stability offered the highest throughput to date. There is still great room for improvement for this technology to scale to industry. It is expected that multi-needle spinneret arrays (see FIG. 8C) or motorized stages capable of higher speed can further improve fabrication times by enabling higher solution flow rates.

The ability to use multiple independent spinnerets during solution electrowriting further enables users to incorporate multiple materials into a single conduit. The ease of use and the modular-style setup of a near-field electro-spinning apparatus enables addition of a second syringe pump that can be used to incorporate fibers into a scaffold that are electrospun from an entirely separate polymer solution. Stereolithography 3D-printing is incapable of simultaneously depositing multiple materials, and extrusion-based 3D printers—commonly used for multi-material deposition—suffer from low resolution.

The ability to incorporate multiple materials during solution electrowriting, enables users to create scaffolds with unique degradation characteristics. These additional spinnerets can be connected to an entirely separate solution reservoir, thus enabling fabrication of what can be considered ‘hybrid’ conduits. As a result, a patterned conduit can be fabricated consisting of two interwoven polymer fibers degrading over different time scales. For instance, solution electro-spinning was demonstrated with solutions containing PCL or PET. If these two polymers were solution electrospun simultaneously from separate solution reservoirs the resulting conduit would consist of both PCL fibers (which degrade in vivo typically within −6 months), and PET fibers (which degrade very slowly over many years). This unlocks a unique feature for patterned conduits especially in the realm of tissue engineering. The fast degrading components degrade to make way for regenerating tissue, while the slow degrading components remain to provide structural support. Incorporation of additional polymers with intermediate degradation rates could result in a scaffold that degrades even more gradually over time. It is readily envisioned that this approach can be expanded depending on the application.

Polymers used to make chronic implantable biomedical devices have to be stable in biological environments so that the devices perform their functions for a period that can be many years. On the other hand, polymers for tissue engineered implants may need to degrade within a time frame that is comparable to the tissue healing processes (weeks to years). Polymers for drug delivery applications may need to degrade within days to years.

The ability to use multiple independent spinnerets during solution electrowriting, further enables users to create scaffolds with unique drug delivery characteristics. It may be desirable to have one solution reservoir loaded with a drug and the other reservoir loaded with a second drug that may not be soluble/compatible with reservoir #1. This provides a way to incorporate two drugs into a scaffold that would not have been able to combine otherwise. A user could also include one drug in a solution that contains a faster degrading polymer and leave the second solution (with slower degrading polymer) drug free. This is a creative way to tune drug release to a desired time frame determined by polymer degradation rates. To expand on that point, the user could also fine-tune the total amount of drug loaded into the scaffold by altering the ratio of the flow rates of both solutions. There are many iterations of this approach that are easy to imagine.

Previously, melt electrowriting and 3D printing were the most viable options for creating tubular biomaterial conduits composed of fibers or struts when fiber patterning and high degrees of porosity are desired. Although solution electrowriting had been demonstrated in previous studies, no efforts had been made to expand the technique for tubular conduit fabrication, or better understand the inner workings and versatility provided by the technique. Presented herein is a systematic investigation of control of scaffold size, geometry, patterning, porosity, and fusion through simple steps without the need for post-processing. It is expected that the knowledge gained here will expand the horizon of solution electrowriting and more precisely controlled fabrication processes.

The disclosure described herein provides a relatively inexpensive and simple way to improve control during electrospun fiber tubular scaffold fabrication. Solution electrospun systems and methods are expected to be useful for producing fibrous biomaterials conduits used as tissue engineered grafts with tunable mechanical properties. Tubular structure is one of the most common in various organs of lifeforms on earth spanning the plant to the animal kingdom. For example, in the human body, GI track, reproductive, urinary, vascular, lymphatic, nerve, and bone all contain tubular structures. Alternately, such biomaterials may be in the form of fibrous mats of various shapes and curvatures produced from such fibrous tubular conduits. Solution electrospun systems and methods are also expected to find widespread usage industrially. Such industrial products may be in the form of fibrous tubular conduits of various shapes or sizes or fibrous mats of various shapes and curvatures produced from such fibrous tubular conduits.

Synthesis of Palmitate Functionalized poly(glycerol sebacate) (PGSP) pre-polymer. All chemicals were used as received (Table 4).

TABLE 4

| Product | Vendor | Location |
|---|---|---|
| 1,1,1,3,3,3-Hexafluoroisopropanol | Oakwood | Estill, SC |
| | Chemical | |
| PET powder (ES306031) | Goodfellow | Coraopolis, PA |
| SGE 5 mL Fixed Luer Lock Tip | Scientific | Palmer, MA |
| Glass Syringes | Instrument | |
| | Services | |
| 22G and 30G blunt-tip needles | Component | Sparta, TN |
| | Supply | |
| | Company | |
| Carbon Conductive Tape | Ted Pella | Redding, CA |
| SEM Pin Stub Mounts | | |
| Stainless steel mandrels (various | McMaster Carr | Elmhurst, IL |
| diameters Polycaprolactone (PCL) | Millipore Sigma | St. Louis, MO |
| Dichloromethane (DCM) | | |
| Glass scintillation vials (20 mL) | | |
| Anisole | TCI Chemicals | Montgomeryville, |
| Palmitic Anhydride | | PA |
| Sodium Hyaluronate | Fisher | Waltham, MA |
| | Scientific | |
| Poly(glycerol sebacate) (PGS, | Secant Group | Quakertown, PA |
| Regenerez ®) | LLC | |
| 1,4-Dioxane (Anhydrous) | Spectrum | New Brunswick, |
| | Chemical | NJ |
| | MFG Corp. | |
| Hexanes (ACS Grade) | Pharmco-Aaper | Brookfield, CT |
| Acetone (ACS Grade) | | |

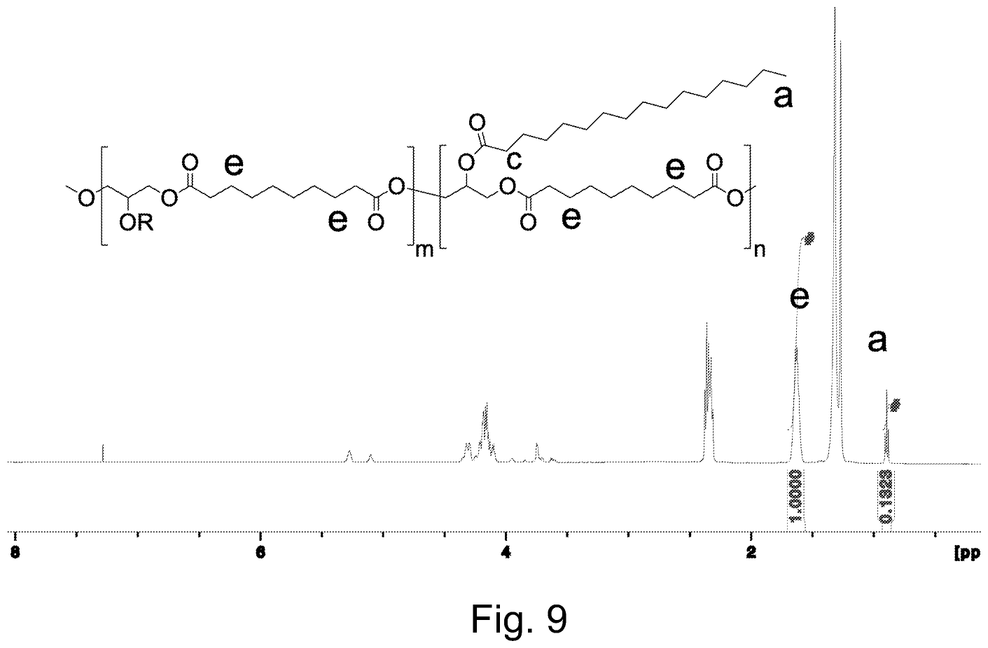

NMR. The degree of modification of PGS with palmitate was determined by proton NMR analysis (500 MHz, Bruker) to be approximately 18 mol. % (FIG. 9). Gel permeation chromatography (Malvern Panalytical OMNISEC GPC system, Malvern Instruments Ltd, UK) analysis determined the number average and weight average molecular weights to be approximately 7,100±675 Da and 237,670±643 Da with polydispersity of 33.66±3.22.

$$\frac{3 {H}_{a} n}{4 {H}_{e} ( n + m )} = \frac{0.1323}{1.} \qquad ( Equation 1 )$$

where n+m=1. The actual composition of palmitate pendants (n) is determined to be approximately 18 mol. %.

Sacrificial Layer for Scaffold Removal. Prior to solution electrowriting, all electro-spinning mandrels were coated with a thin layer of sodium hyaluronate (HA) to create a water-soluble sacrificial layer. Submerging collection mandrels in DI water after solution electrowriting for 1-2 hours dissolved the HA layer to facilitate scaffold removal. HA was dissolved in MilliQ water (1% m/V) overnight under rotation at room temperature then held at 4° C. until further use. To create the sacrificial layer, 1% HA solution was first drawn into a syringe then slowly dispensed onto a rotating mandrel to create a uniform layer. While continuing to rotate the mandrel, the water in solution was then evaporated with a heat gun.

Electro-spinning Device. The near-field electro-spinning device used in the present disclosure consists of many of the same components used in conventional electro-spinning, which include a high-voltage power supply (Gamma High Voltage Research, Ormond Beach, FL), digital overhead stirrer (Southwest Science, Trenton, NJ), syringe pump (New Era Pump Systems, Farmingdale, NY), and an electrically grounded fiber collection mandrel (FIG. S1). To enable uniaxial translation of the electro-spinning spinneret, the syringe pump used to dispense polymer solution was mounted onto a motorized positioning stage (Velmex, Bloomfield, NY). The VXM COSMOS software provided with the motorized stage requires the user to input values to determine the stage and range of the motorized stage. Changing stage range enables precise control over the length of the resulting solution electrowritten graft. By changing stage speed, as well as mandrel rotational speed, the fiber winding angle can be tightly controlled.

Electro-spinning. During the present disclosure, many combinations of electro-spinning solutions and parameters were tested to explore the effects on the resulting solution electrowritten conduit. PGSP/PET scaffolds were all electrospun from solutions containing 20% m/V PGSP and 20% m/V PET. All PCL electro-spinning, regardless of solvent, were conducted using solutions containing 25% m/V PCL. All electro-spinning solutions were prepared at least 48 hours prior to electro-spinning. For PGSP/PET solutions PET was first added to HFIP and agitated on a rotating shaker at 37° C. for 24 hours until fully dissolved. PGSP was then added to the solution and agitated for an additional 48 hours at 37° C. before use. Electro-spinning parameters for the various experiments discussed in the manuscript are included in Table 1. All solution electrowriting was conducted with a spinneret-to-mandrel gap distance of 3 mm. It was found that gap distances ranging from 2-8 mm were all feasible with these electro-spinning solutions and fabrication parameters.

PGSP Thermal Crosslink. PGSP is a thermoset elastomer that requires thermal curing to crosslink the prepolymer into an insoluble network. From experiments, PGSP will not crosslink at temperatures below 100° C. After electro-spinning PGSP/PET, the entire collection mandrel with fibers collected was placed in a vacuum oven at 80° C. for 24 hours to remove residual electro-spinning solvent. After 24 hours the temperature was increased to 100° C. for an additional 48 hours.

Hyaluronic acid (HA) Solubilization and Scaffold Removal. Crosslinked PGSP/PET and as-spun PCL scaffolds were released from collection mandrels by first solubilizing the sacrificial HA layer. Mandrels were submerged in DI water at room temperature for 3-4 hours. This allowed sufficient time for water to penetrate the hydrophobic fiber network and solubilize the underlying HA. Scaffolds were then slid off the mandrel. Released scaffolds were then frozen and lyophilized for 24 hours to remove any remaining moisture prior to further analysis.

Scanning Electron Microscopy and Analysis. All scanning electron microscopy (SEM) was conducted using a Tescan Mira3 FESEM. Samples were first mounted on to SEM mounts with conductive carbon tape and sputter coated with a 5 nm thick layer of Au/Pd. Samples were imaged with a 5 kV accelerating voltage at various degrees of magnification.

To quantify the degree of anisole-induced fiber point fusion. One hundred fiber cross points on each of three independently fabricated samples (n=3) for each electro-spinning solution formulation were analyzed. Thus, large numbers of fibers needed to be studied at high magnification. At these high magnifications only 1-3 fiber crossing points were visible in a single field of view. Thus, rather than acquire 1200 total images, each sample was scanned at high magnification and analyze randomly selected fields of view. At each field of view the number of fused and non-fused fiber cross points was recorded until a total of 100 cross points were analyzed for each sample. These data were then used to calculate the percentage of fused fiber cross points in each sample.

Statistical Analysis. All statistical analysis in the present disclosure was conducted using Kyplot software (Version 6.0.1). All data in this manuscript are presented as mean values±standard deviation. Data were compared using a one-way analysis of variance (ANOVA) followed by a Tukey's honest significant difference test (HSD) to compare groups directly to each other. Within each figure, asterisks are used to represent confidence levels *p<0.05, **p<0.01, ***p<0.001.

### Example 2

The following is an example of solution electrowriting systems of the present disclosure, methods of using said solution electrowriting systems, and solution electrowritten products produced using said systems and/or said methods.

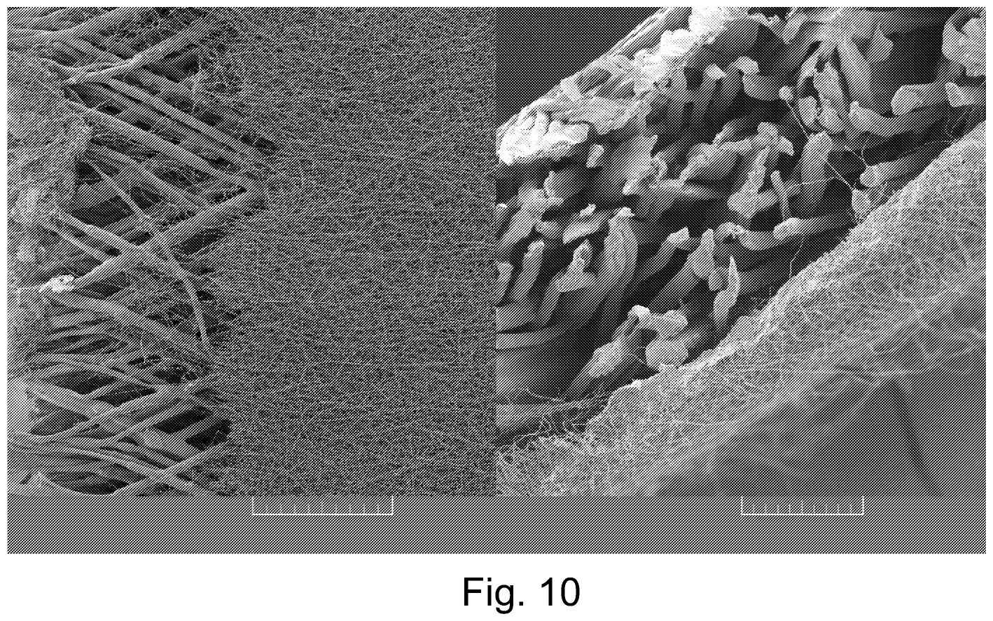

Hybrid solution electrowritten and conventional electrospun conduits are easy to fabricate with a device of the present disclosure. Because the described solution electrowriting device consists of a modified conventional electro-spinner, it is easy to perform solution electrowriting then change fabrication parameters to immediately begin conventional solution electro-spinning with the same solution (or a second solution can be introduced). FIG. 10 shows two SEM micrographs (Left—top view, Right—side view) of a hybrid conduit in which PGSP/PET in HFIP (40% m/V) was solution electrowritten as described in Example 1 to create a tubular conduit. After finishing solution electrowriting, the PGSP/PET solution was replaced with a solution containing 12% m/V of gelatin in the solvent trifluoroethanol. Gelatin was spun with a higher voltage than PGSP/PET (5 kV vs 1.2 kV) to induce whipping instability and sheathed the PGSP/PET microfiber conduit in gelatin nanofibers.

This design was tested because solution electrowritten conduits are porous. When using these grafts as synthetic vascular grafts they will likely leak blood early after implantation. Gelatin is a hemostatic material and the gelatin sheath on this ‘hybrid graft’ can induce hemostasis shortly after implantation. It is expected that this approach may be unique in its ability to enable this type of sequential fabrication where a patterned fibrous tubular conduit and a nanofibrous conduit can be fabricated sequentially without transferring the conduit to a second device. It would also be easy to reverse the solution change and return to solution electrowriting after electro-spinning the nanofibrous layer. This unlocks the ability to make multilayered conduits where each layer can have different patterning and composition to serve different purposes. Further, the nanofibrous gelatin layer does not need to be ‘nanofibrous’. By changing the solution electro-spinning parameters, this outer fiber sheath could also contain microfibers. This layered approach is customizable depending on user input and need.

A conduit with multiple distinct layers that are all fabricated with solution electrowriting can be prepared. Such multilayer PGSP/PET scaffolds were prepared by solution electrowriting at a winding angle of −45° then the spinneret translation speed was changed to deposit a layer of circumferentially oriented fibers over the length of the conduit. Then the original fabrication parameters to used to create a third layer consisting again of fibers with a 45° winding angle. The result was a conduit that had the porosity of a typical solution electrowritten conduit, but had a middle layer that should limit mass transfer, or cell migration in the context of tissue regeneration.

In terms of vascular graft applications, this design holds promise. This approach allows cells from the lumen to migrate into the inner walls of the graft and start to deposit extracellular matrix and build new tissue. This also allows cells from the periphery to do the same. However, the middle circumferential hinders cells from the periphery to cross into the lumen via transmural migration. This can prevent unwanted interactions between cells and allow a neovessel to develop distinct regions reminiscent of the tunica intima, tunica media, and tunica adventitia.

A tracheal graft can be fabricated via solution electrowriting. A tube similar to those discussed already can be fabricated then reinforced with stacked rings of circumferentially oriented fibers on the outside of the tube. This circumferential rings can mimic the rings of hyaline cartilage found in the native trachea. 3D printing approaches have been used to create similar structures. However, with solution electrowriting, the expanded library of usable polymers can allow users to create a tracheal graft with degradation rates and mechanical properties that closely match native trachea.

### Example 3

The following is an example of solution electrowriting systems of the present disclosure, methods of using said solution electrowriting systems, and solution electrowritten products produced using said systems and/or said methods.

Described is a graft that designed to remodel to an autologous structure and to match the compliance of venous end and approximate the compliance of the arterial end. This approach is expected to achieve the long-term patency of successful AV fistulae without the limitations of existing synthetic grafts (restenosis, infection) and allografts/xenografts (immunosensitization, infection).

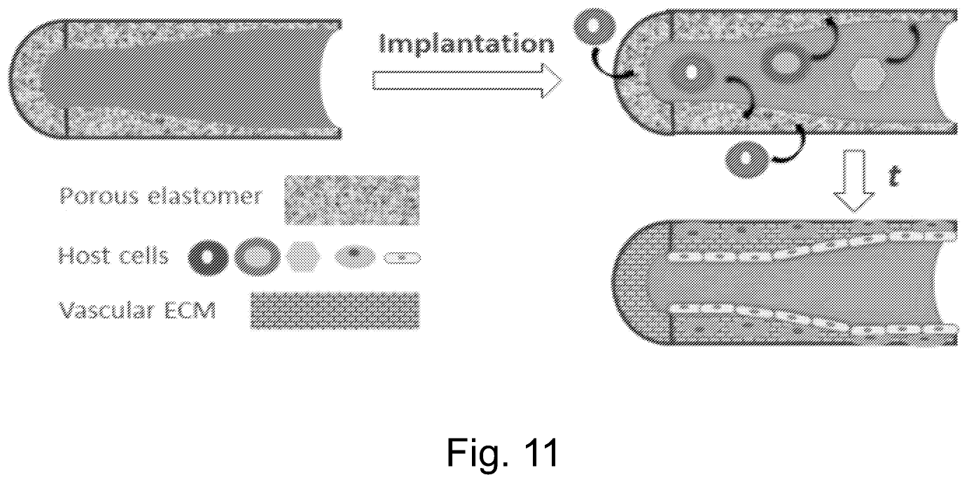

This graft responds to the priority area of vascular access and drainage constructs. A hemocompatible surface is a core technology for vascular access and is essential to artificial kidneys. A hemocompatible surface remains elusive after decades of research. The present disclosure describes a vascular graft that transforms from a synthetic tube to an autologous vascular conduit. This transformation will benefit the whole body as it avoids chronic exposure of host cells to foreign materials. This transformation has been demonstrated in animal models using an elastic and degradable synthetic graft. FIG. 11 illustrates an access graft designed for the specific needs of hemodialysis. The requirement of high flow dictates direct linkage of an artery to a vein, creating a conundrum where current solutions invariably cause supraphysiologic blood pressure and shear stress, and turbulent flow in the vein. FIG. 12 illustrates a graft designed with a transition zone that gradually increases the diameter and reduces wall thickness from the arterial to the venous zone. This gradual transition will lower the pressure and reduce the turbulence in the outflow vein. The compliance of the thinner and elastic venous end of the graft will match that of the vein. It is expected that the host-remodeled graft will be a 100% autologous tissue with an endothelialized lumen and a vessel wall made of vascular extracellular matrix and mural cells. Consequently, a higher patency rate is expected because of the reduction of stenosis, matching compliance, and increased hemocompatibility.

Hemodialysis patients desire a vascular access that can be used immediately and is durable. A graft was thus designed that can transform into patient's own vascular conduit with matching compliance and endothelialized lumen. It is believed that the major causes of synthetic graft failure are the prolonged presence of foreign materials and compliance mismatch. The latter is true even for allograft and fistula because veins are far more compliant than arteries. Based on this, an arteriovenous (AV) graft was designed (FIG. 12) featuring: 1. Fully biodegradable synthetic polymers with established biocompatibility. 2. An arterial-like segment that anastomoses with the patient's artery; the compliance will be slightly higher than that of the native artery. 3. A transition zone with gradually increasing diameter accompanied by reduction in wall thickness. and 4. A venous zone matching the compliance of the vein that reduces the mechanical overload of the vein. The gradual increase in diameter will reduce turbulence in the outflow vein. It is estimated that the portion of the graft with over 0.5 mm-thick wall can be cannulated. Allocating a generous 1 cm of the arterial zone for suturing, more than 17 cm of the graft is a tough ‘canulation zone’. Furthermore, the graft is made of elastic fibers that bring two inherent benefits: no kink upon bending and immediate cannulation. Needles push the elastic fibers apart, which recoil and seal the hole upon needle withdrawal.

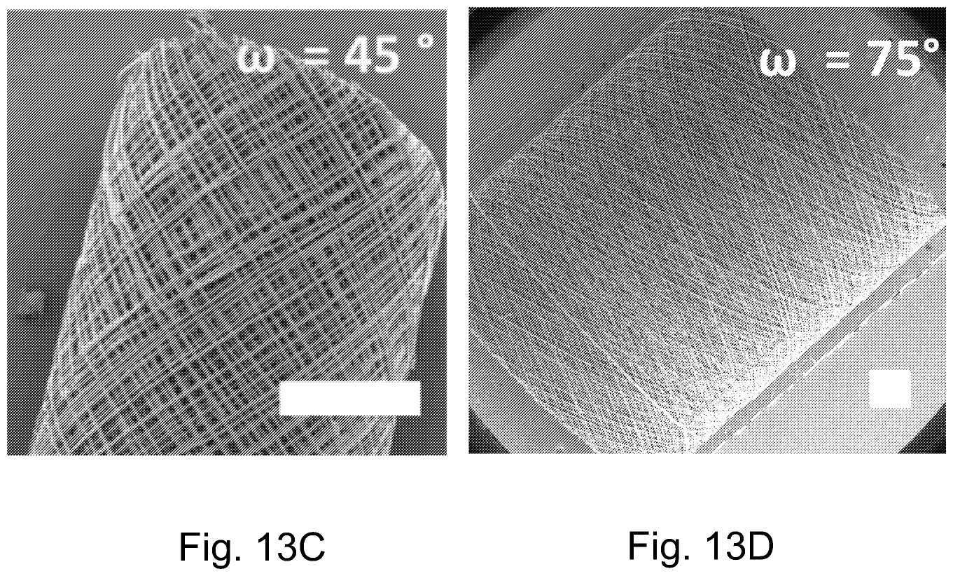

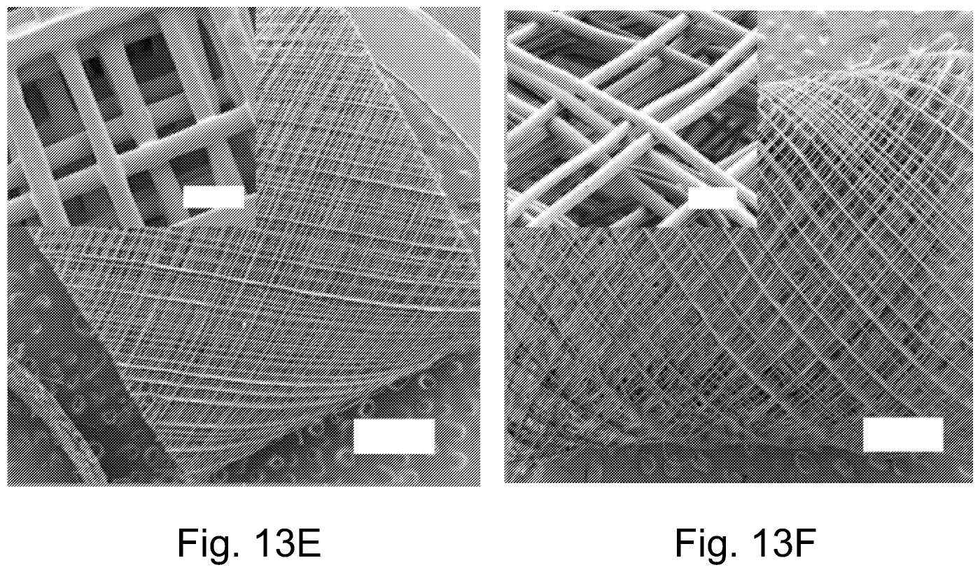

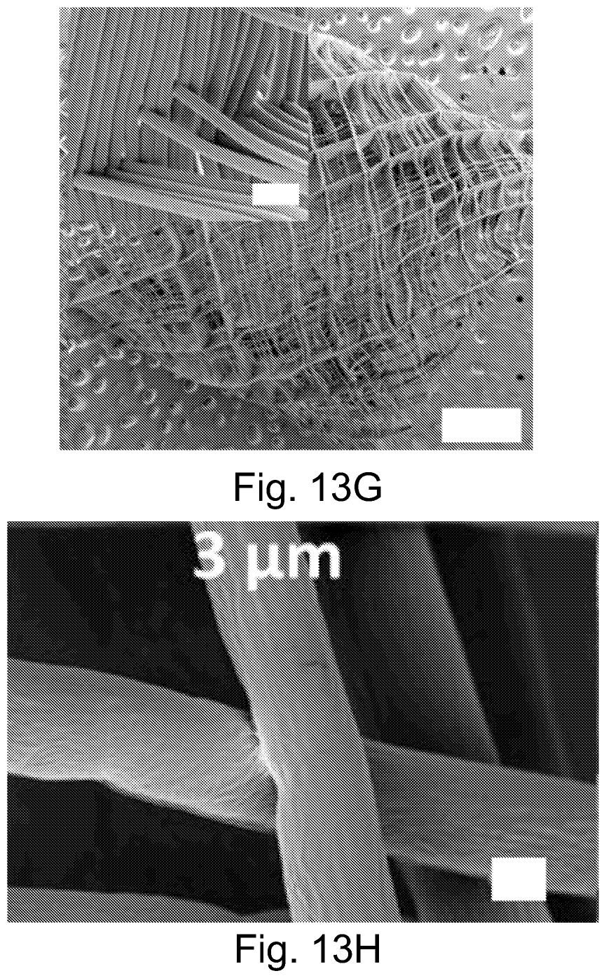

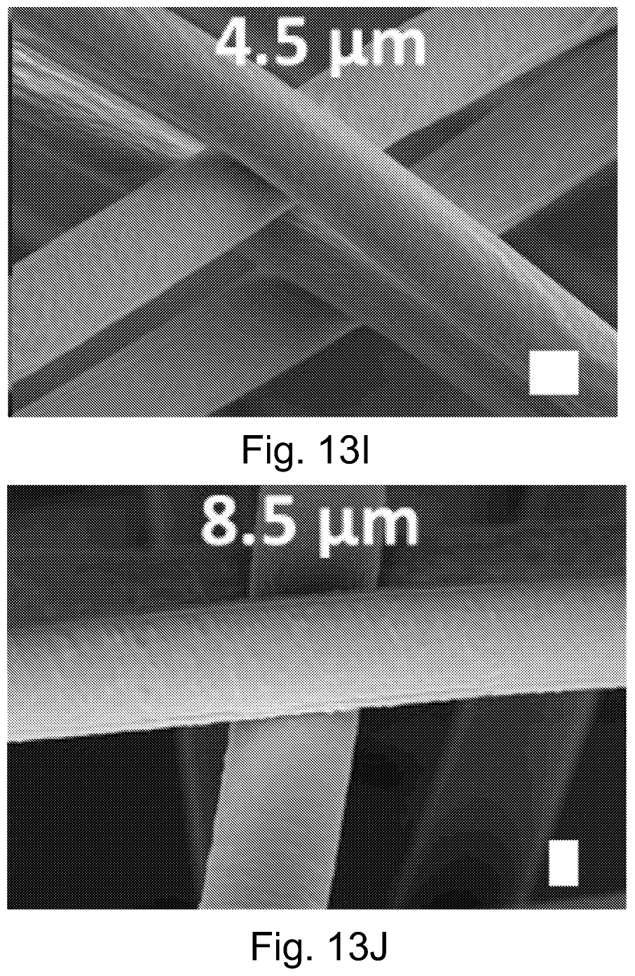

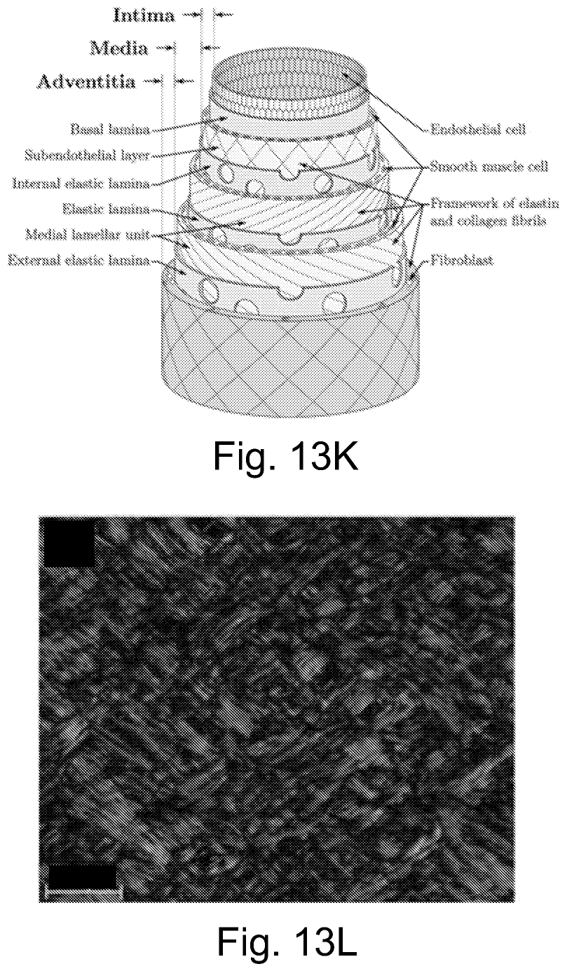

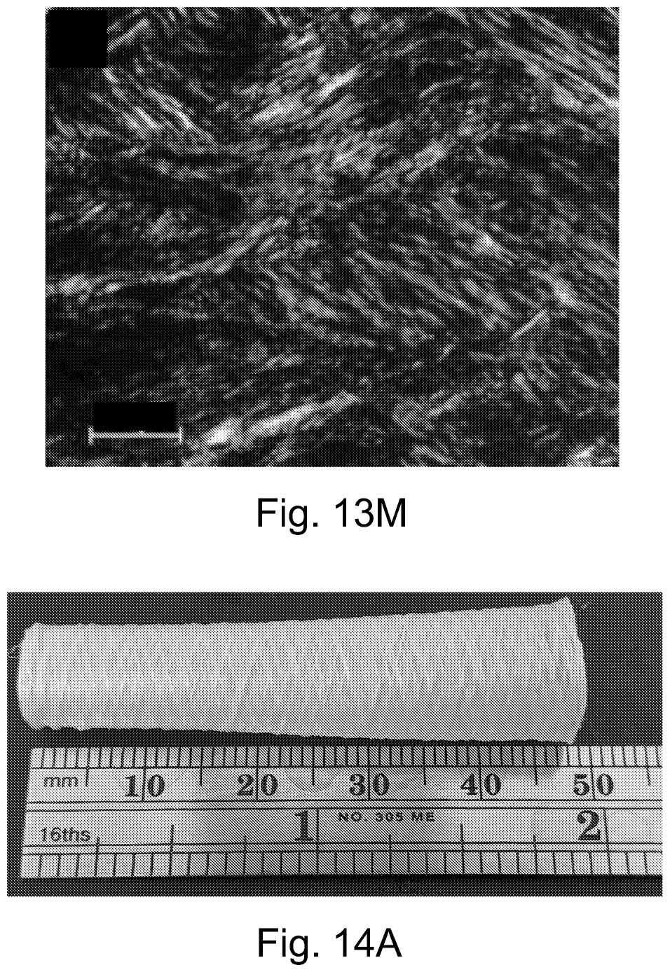

The graft is an elastic porous tube made of poly(glycerol sebacate) palmitate (PGSP, 90% of wall thickness) surrounded by a slow-degrading sheath of polycaprolactone (PCL, 10% of wall thickness). The graft was made using solution electrowriting systems and methods described herein. Solution electrowriting allows precision control of fiber diameter, spacing and winding angle (FIG. 13). These 3 parameters were controlled in each layer through software on a computer-controlled graft fabrication device. For example, a dense fibrous layer can be placed anywhere in the graft to limit mass transfer. Polymer type can be switched when desired. The manufacturing process can be scaled up by using a multi-needle spinneret (FIGS. 8B-8C). The process is semi-automated and is expected to be fully automated.

After implantation, PGSP begins to degrade into small molecules of glycerol, palmitate and sebacate. Glycerol and palmitate are molecules that the body uses readily to build lipid membranes. Sebacate has been investigated as a carbon source to produce ATP for bed ridden patients. PCL has a long clinical history and is known to be safe and slow degrading (nearly a year after implantation). PCL was engineered into the graft as a ‘safety net’ ensuring the graft will not rupture in cases of slower tissue regeneration rate. As the scaffold breaks down gradually, the patient's own cells produce an autologous extracellular matrix and build up a new vascular conduit in its place.

The graft degrades as host tissues replace and transform the graft into an autologous vascular conduit. This avoids long-term exposure to foreign materials. The autologous endothelium will improve hemocompatibility. The autologous nature of the remodeled graft will enable immune surveillance and reduce infection.

To match the venous compliance, the venous zone was designed to have a thin wall. On the other hand, a thicker wall of the arterial zone will reduce the compliance to approach that of the artery. The asymmetric design of the graft increases compliance matching of the shunt to its two distinct anastomoses from the very beginning (FIG. 12). It is believed that this unique design will increase the long-term patency of the graft.

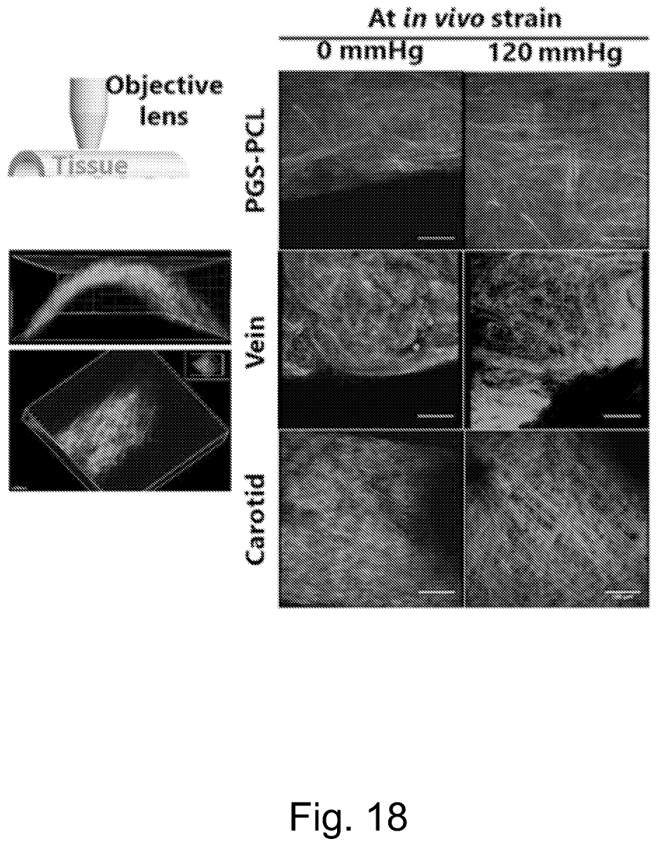

The graft is made of elastic biodegradable polymers based on poly(glycerol sebacate) (PGS). The modulus of the polymer is in the range of human blood vessels, approximately 1000× softer than PTFE. Fine control over the mechanical properties of the elastomer can be achieved by adjusting monomer ratio, polymerization parameters and substitution with palmitate. This allows the graft to closely match the biomechanical properties of the blood vessel. The elasticity of the graft promotes elastic fiber formation. In adult patients, elastin synthesis is limited. However, it is believed that the capability of the graft to change dimensions under physiological blood pressure will still allow the host cells to produce undulating collagen fibers, as was observed in animal studies. This will enable their recruitment under mechanical loads (as seen in FIG. 18), transforming the graft into a compliant vascular conduit than what would otherwise be a stiff conduit resulting from inflammation driven collagen deposition.

It is expected that the graft will have immediate impact on vascular access. The graft requires no maturation time and can be cannulated soon after implantation. It is expected that the synthetic graft will transform into an autologous vascular conduit within 6-12 months based on animal models. Thus, the graft will be “personalized” and will have a reduced risk of infection. Therefore, the product combines the benefits of the AV fistula and leading synthetic grafts, potentially outperforming both. Transformation into an autologous gradual transition between the high-pressure artery and low-pressure vein will alleviate the detrimental impact of compliance mismatch, thrombogenicity, and chronic inflammation. It is therefore expected to provide a significant improvement in patency and longevity of the graft. It is also predicted that the graft will eliminate the unsightly aneurysmal and enlarged vein that patients despise. The synthetic nature of the graft allows easier industrial scaleup and distribution than competing products that require biologics and cells. Therefore, the cost benefit will be significant to the patients and the healthcare system.

Graft design and optimization. The computer program and the mandrel used in the solution electrowriting systems and methods of the present disclosure can be modified to produce full-length (30 cm) arteriovenous grafts. A series of products with different sizes can be easily produced. In the current design (FIG. 12), the arterial zone is 2 cm long, inner diameter 4 mm, wall thickness 1 mm, the transition zone is 26 cm long, the venous zone is 2 cm long, inner diameter 6 mm, wall thickness 0.2 mm. Leaving 1 cm of the arterial zone as margins for suturing, 17.25 cm of the graft will have a wall thickness over 0.5 mm. This will be the canulation zone. The solution electrowriting systems and methods of the present disclosure can be fully automated. Automation increases rigor and reproducibility.

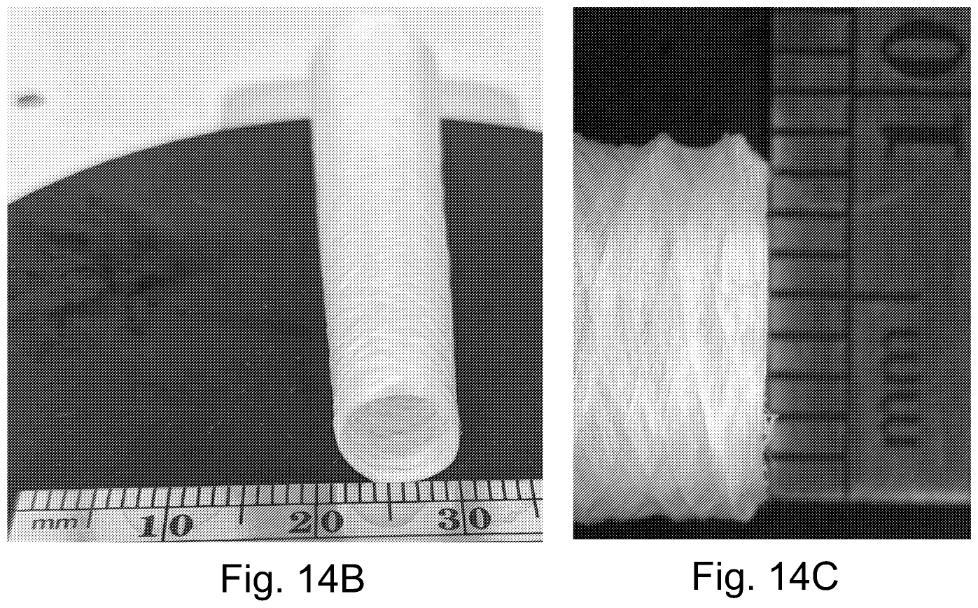

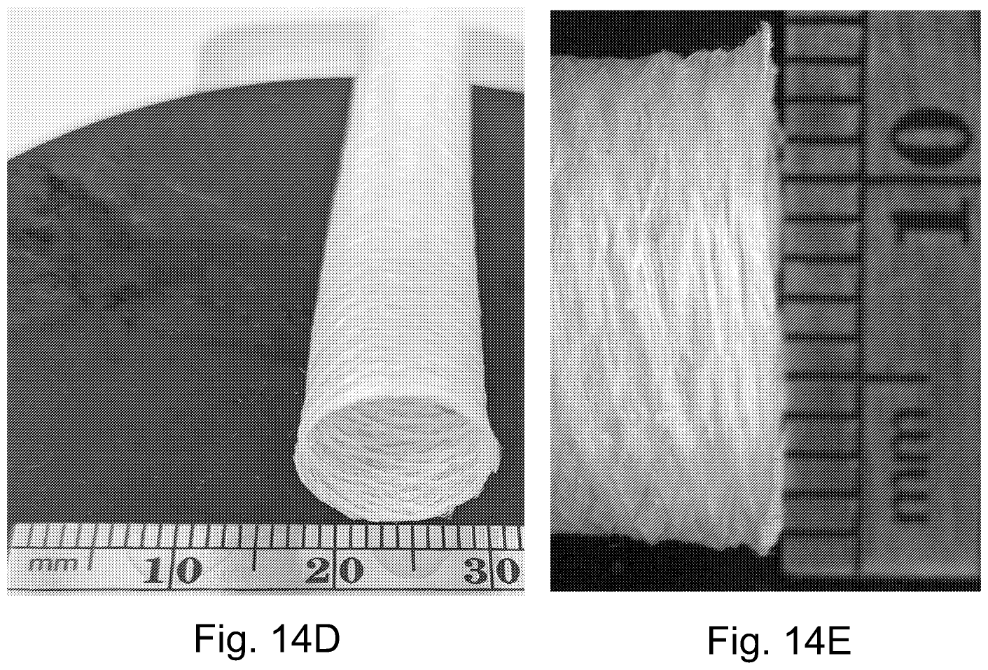

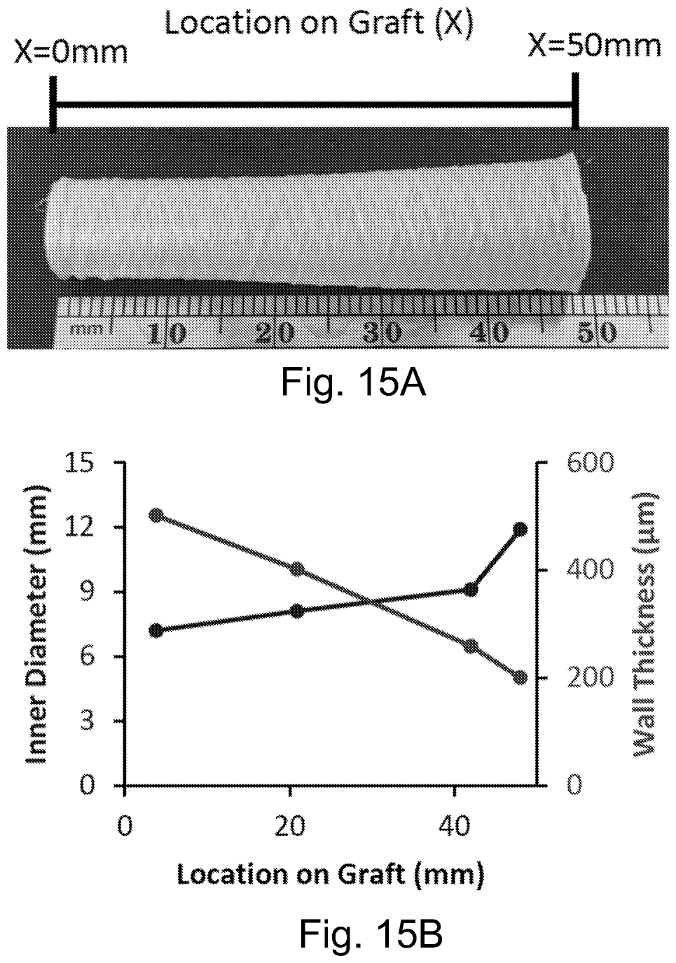

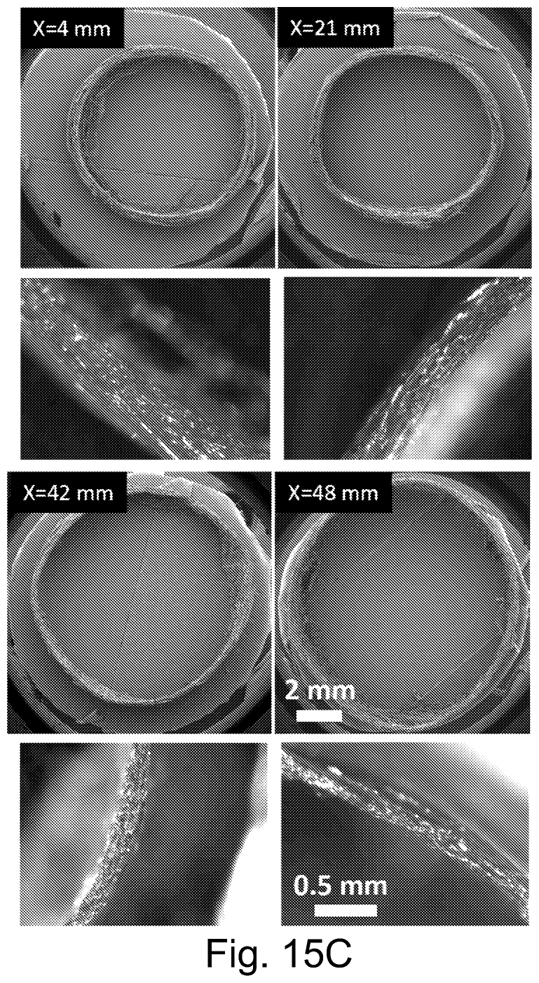

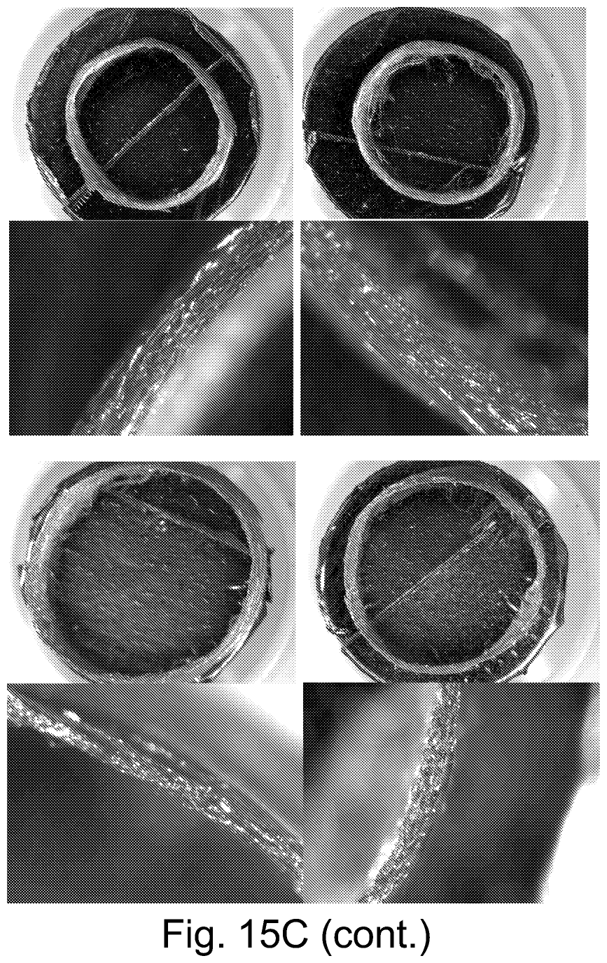

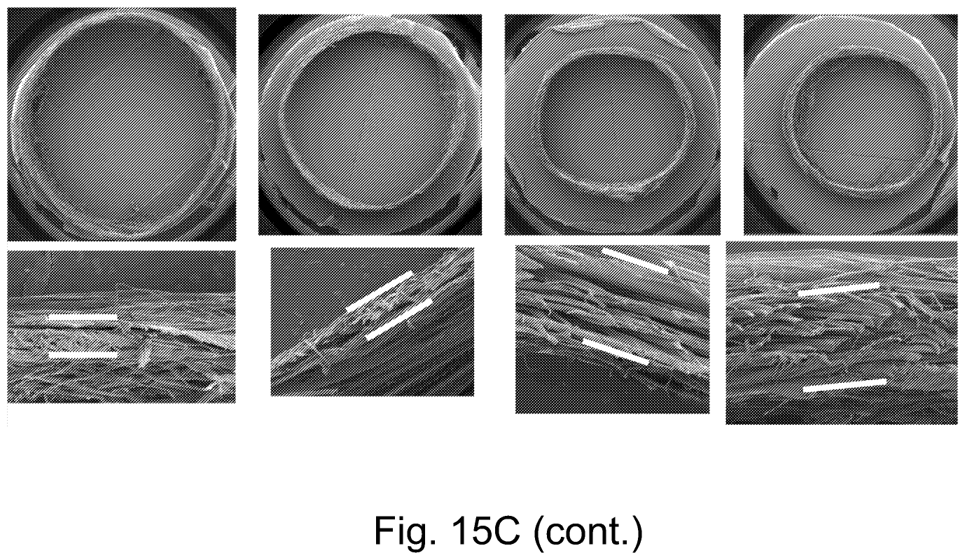

Graft fabrication. PGS grafts specifically designed for engineering soft elastic tissues (FIG. 13). Moreover, solution electrowriting systems and methods of the present disclosure were used to fabricate the key component of the graft, the transition zone that bridges the arterial and the venous zone (FIGS. 14-15). A commercially available tapered mandrel with larger than desired diameter was used for proof-of-principle. A tapered mandrel can be lathed complete with all three zones.

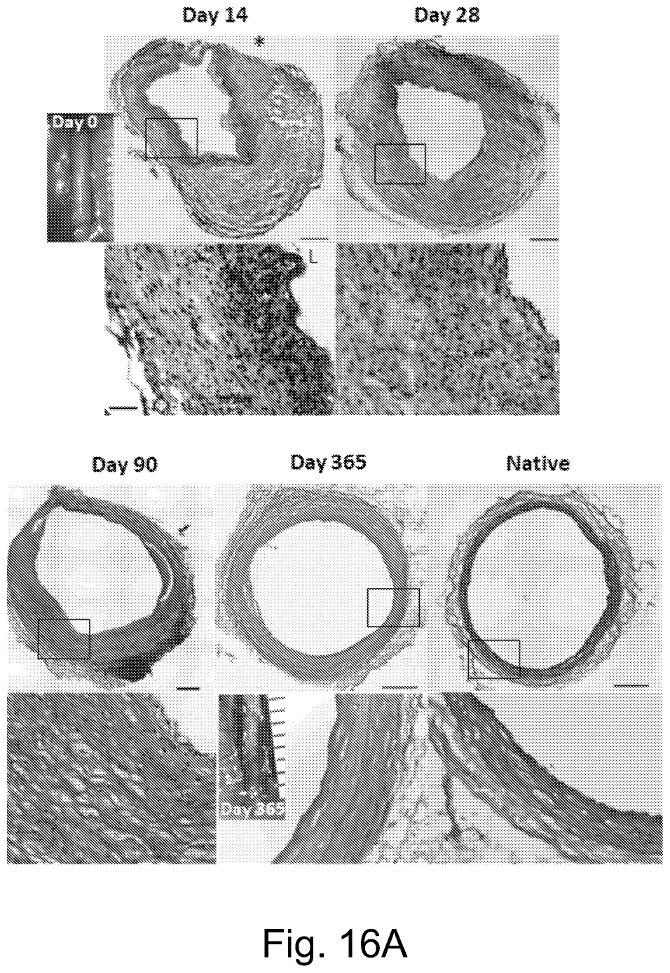

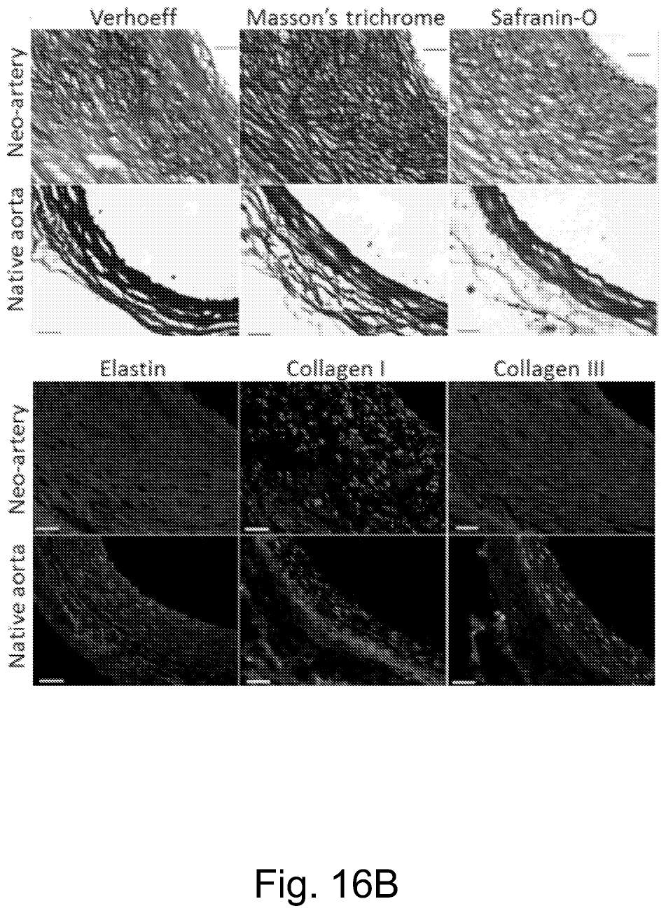

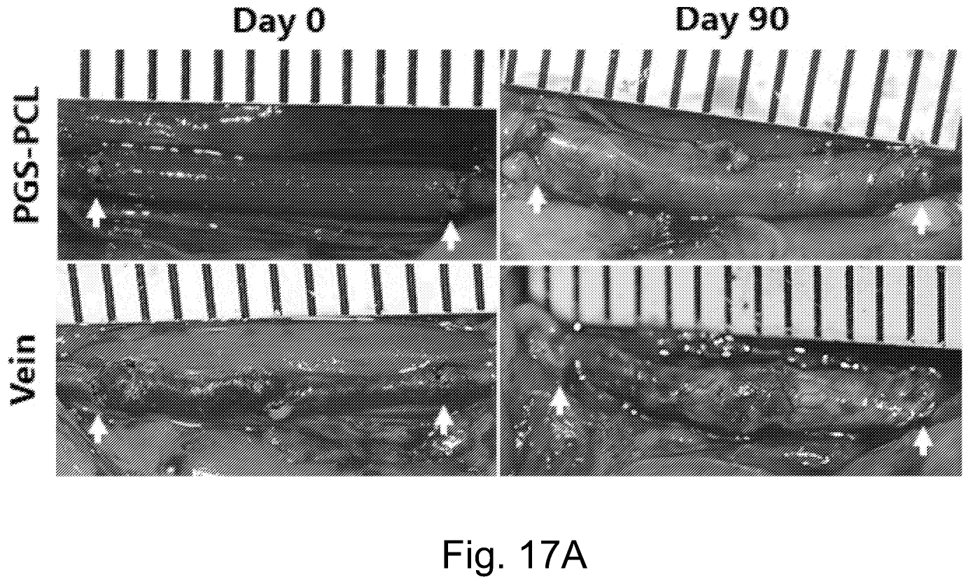

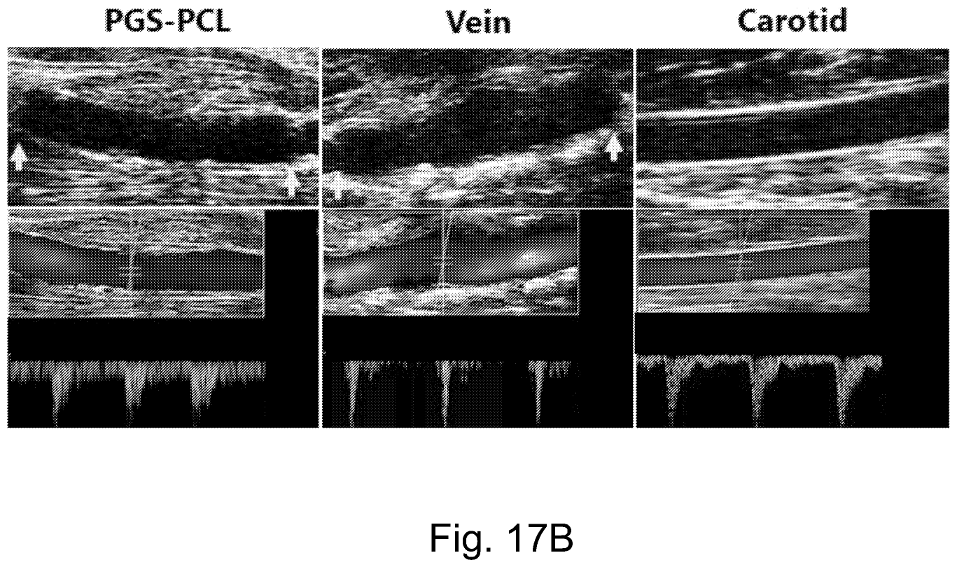

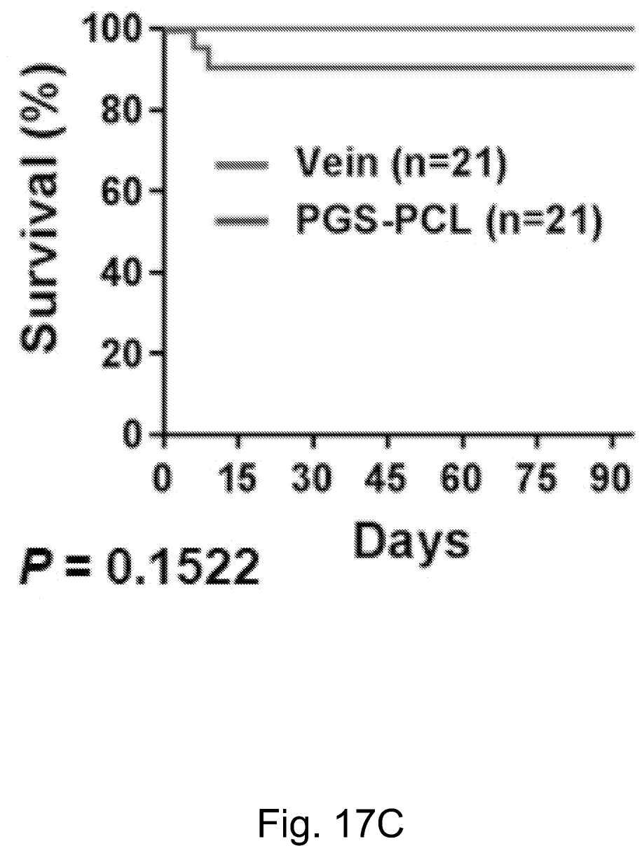

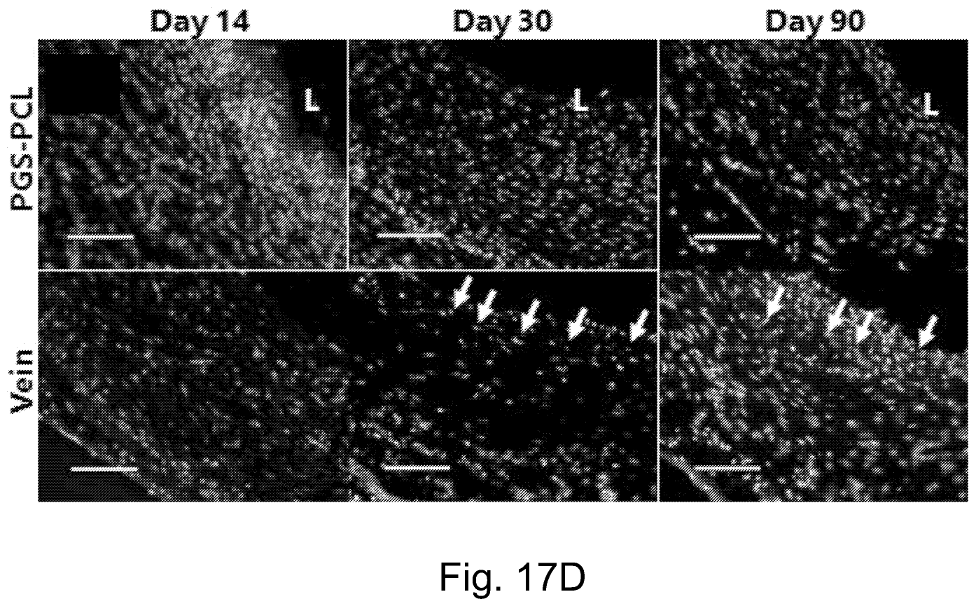

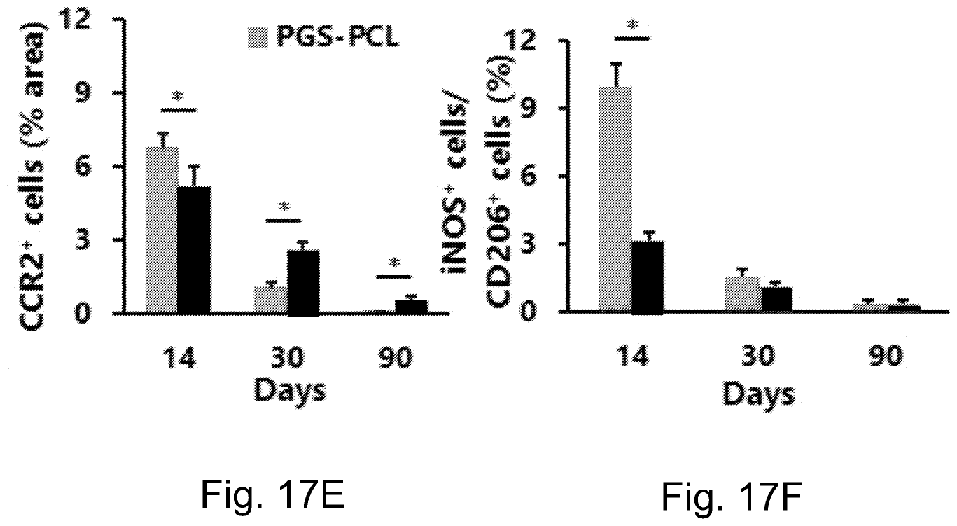

In vivo transformation in rat models. Iterations of PGS-based grafts were tested in rodents. The transformation of PGS grafts into autologous conduit was demonstrated in a rat abdominal aorta model. The remodeled graft resembled the composition and mechanical properties of native aorta (FIG. 16). Next a rat carotid artery model was used, a more demanding environment for thrombosis. When compared head-to-head with autologous vein grafting, inflammation in the PGS-PCL composite graft resolved more rapidly and induced less smooth muscle cell proliferation than vein grafts (FIG. 17-18), indicating less likelihood of developing stenosis later.

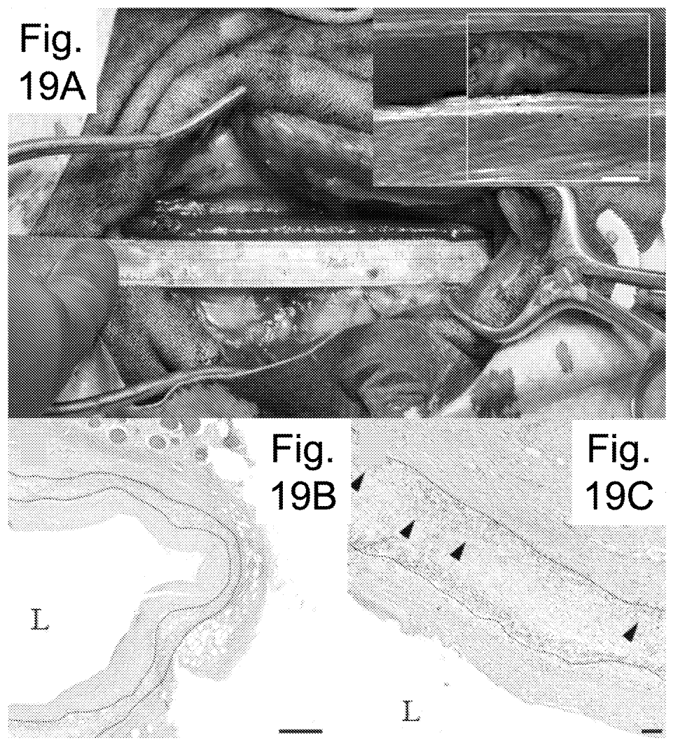

Preliminary testing in a sheep carotid model. PGS-PCL grafts were successfully implanted in sheep (FIG. 19). Interposition implantation in sheep carotid artery demonstrated good handling and suturability. Duplex ultrasound examination at 2 weeks showed a patent graft. Histological examination showed excellent host cell infiltration and endothelialization at anastomoses.

Testing AV grafts in a sheep model. Both pig and sheep are good models to test AV grafts. Sheep have long and readily accessible neck vessels, making them desirable for testing longer grafts. Furthermore, sheep coagulation system is similar to that in humans. AV access was achieved using a sheep carotid to jugular straight AV graft model using both male and female 7-year-old sheep. Grafts were placed as an end to side anastomosis to the proximal carotid artery and end to side anastomosis to the distal jugular vein. A preliminary study in sheep carotid interposition grafting showed excellent host cell infiltration within 15 days (FIG. 19).

Although the present disclosure has been described with respect to one or more particular example(s), it will be understood that other examples of the present disclosure may be made without departing from the scope of the present disclosure.

1. A method of making a solution electrowritten fibrous product, the method comprising:
providing a solution electrowriting system comprising:
one or more nozzle(s);
a material supply system comprising one or more reservoir(s) fluidically coupled to the nozzle(s) and configured to supply one or more fluid stock(s) to the nozzle(s) thereby ejecting one or more jet stream(s) of the fluid stock(s) from the nozzle(s);
a collector system configured to collect one or more fiber(s) formed by the jet stream(s) ejected from the nozzle(s); and
one or more power source(s) configured to provide one or more electric potential(s) to each of the nozzle(s) and, optionally, to the collector system, thereby providing one or more electric potential difference(s) between the collector system and each of the nozzle(s);
wherein each fluid stock comprises a solution comprising at least one first solvent and, optionally, at least one second solvent, and one or more material(s) configured to form at least a portion of a fiber upon ejection of the jet stream(s) of the fluid stock(s) from the nozzle(s), wherein the material(s) is/are dissolvable in at least one of the solvent(s) to form a solution;
ejecting the fluid stream(s) of the fluid stock(s) from the nozzle(s) to form the fiber(s);
collecting the fiber(s) with the collector system to form a fibrous product comprising one or more fiber(s) arranged in a predetermined pattern; and
releasing the fibrous product from collector system,
wherein a desired fiber fusion and/or a desired fiber stacking is observed in the fibrous product.

2. The method of claim 1, further comprising, after the collecting and/or the releasing, heating and/or drying the fibrous product.

3. The method of claim 1, wherein the fluid stock(s) comprise(s) at least one first solvent and at least one second solvent, and wherein the at least one first solvent has a boiling point of less than about 80° C., and wherein the at least one second solvent has a boiling point of at least about 80° C. or greater.

4. The method of claim 1, wherein the fluid stock(s) comprise(s) at least one first solvent and at least one second solvent, and wherein the boiling point of the at least one second solvent is from about 10° C. to about 200° C. higher than the boiling point of the at least one first solvent.

5. The method of claim 3, wherein the at least one first solvent is chosen from diethyl ether, dichloromethane (DCM), acetone, 1,1,1,3,3,3-hexafluoroisopropanol (HFIP), chloroform, methanol, tetrahydrofuran (THF), trifluoroethanol (TFE), ethanol, acetonitrile, cyclohexane, benzene, ethyl acetate, hexane, trifluoroacetic acid, isopropanol, and any combination thereof; and/or wherein the at least one second solvent is chosen from water, dioxane, toluene, pyridine, N,N-dimethylformamide (DMF), anisole, dimethyl sulfoxide (DMSO), 1,2-dichloroethane, triethylamine, heptane, butanol, acetic acid, xylene, diglyme (diethylene glycol diethyl ether), and any combination thereof.

6. The method of claim 3, wherein the volume ratio of the at least one first solvent to the at least one second solvent is from about 1:99 to about 99:1.

7. The method of claim 3, wherein the fibrous product comprises a plurality of fusion points between respective portions of at least two adjacent intersected fibers, such that for each fusion point, a bottom surface of a first fiber is bonded to a top surface of a second fiber.

8. The method of claim 7, wherein the plurality of fusion points between adjacent intersected fibers is observed at an average frequency of from about 5% to about 99%.

9. The method of claim 1, wherein the fluid stock(s) comprise(s) at least one first solvent having a boiling point of from about 70° C. to about 120° C.

10. The method of claim 9, wherein the at least one first solvent is chosen from trifluoroethanol (TFE), ethanol, acetonitrile, cyclohexane, benzene, ethyl acetate, hexane, trifluoroacetic acid, isopropanol, water, dioxane, toluene, pyridine, and any combination thereof.

11. The method of claim 9, wherein the fibers comprise a plurality of fusion points between respective portions of at least two adjacent intersected fibers, such that for each fusion point, a bottom surface of a first fiber is bonded to a top surface of a second fiber.

12. The method of claim 11, wherein the plurality of fusion points between adjacent intersected fibers is observed at an average frequency of from about 5% to about 99%.

13. The method of claim 1, wherein the fluid stock(s) comprise(s) at least one first solvent and at least one second solvent, and wherein the at least one first solvent has a dipole moment of from about 1.5 D to about 4.2 D and the at least one second solvent has a dipole moment of from about 0 D to less than about 1.5 D.

14. The method of claim 1, wherein the fluid stock(s) comprise(s) at least one first solvent and at least one second solvent, and wherein the dipole moment of the at least one first solvent is about 20% or more greater than the dipole moment of the at least one second solvent.

15. The method of claim 14, wherein: the at least one first solvent is chosen from dichloromethane, tetrahydrofuran (THF), pyridine, trifluoroethanol, acetone, ethanol, methanol, N,N-Dimethylformamide, dimethyl sulfoxide (DMSO), isopropanol, water, ethyl acetate, trifluoroacetic acid, 1,1,1,3,3,3-hexafluoroisopropanol, 1-butanol, 1,2-dichloroethane, acetic acid, diglyme, acetonitrile, and any combination thereof; and/or the at least one second solvent is chosen from cyclohexane, hexane, benzene, toluene, dioxane, diethyl ether, chloroform, anisole, triethylamine, heptane, xylene, and any combination thereof.

16. The method of claim 14, wherein the volume ratio of the at least one first solvent to the at least one second solvent is from about 1:99 to about 99:1.

17. The method of claim 14, wherein for each axial direction of the fibrous product, adjacent fibers of different layers are aligned and are vertically stacked.

18. The method of claim 1, wherein the fluid stock(s) comprise(s) at least one first solvent having a dipole moment of from about 1.5 D to about 4.2 D.

19. The method of claim 18, wherein the at least one first solvent is chosen from dichloromethane, tetrahydrofuran (THF), pyridine, trifluoroethanol, acetone, ethanol, methanol, N,N-Dimethylformamide, dimethyl sulfoxide (DMSO), isopropanol, water, ethyl acetate, trifluoroacetic acid, 1,1,1,3,3,3-hexafluoroisopropanol, 1-butanol, 1,2-dichloroethane, acetic acid, diglyme, acetonitrile, and any combination thereof.

20. The method of claim 18, wherein for each axial direction of the fibrous product, adjacent fibers of different layers are aligned and are vertically stacked.

21. The method of claim 1, wherein the fluid stock(s) further comprise(s) a conductive agent.

22. The method of claim 21, wherein the conductive agent is chosen from a salt, a conductive polymer, and any combination thereof.

23. The method of claim 22, wherein the salt is present in the fluid stock(s) at from about weight % to about 10 weight %, based on the total weight of the material(s), or wherein the conductive polymer is present in the fluid stock(s) at from about 0.1 weight % to about 100 weight %, based on the total weight of the material(s).

24. The method of claim 21, wherein for each axial direction of the fibrous product, adjacent fibers of different layers are aligned and are vertically stacked.

25. The method of claim 1, wherein the electric potential applied to the nozzle(s) is from about 50V to about 8 kV.

26. The method of claim 25, wherein for each axial direction of the fibrous product, adjacent fibers of different layers are aligned and are vertically stacked.

27. The method of claim 1, wherein the fibrous product comprises one or more layer(s) each comprising one or more group(s) of fibers optionally aligned in one or more axial directions of the fibrous product within and/or between the layer(s).

28. The method of claim 27, wherein the group(s) of fibers is/are uniaxially, biaxially, or multi-axially oriented within and/or between the layer(s).

29. The method of claim 27, wherein each group of fibers has a substantially constant winding angle, relative to the longitudinal axis of the fibrous product.

30. The method of claim 29, wherein the substantially constant winding angle comprises an angle between from about 1° to about 89°, relative to the longitudinal axis of the fibrous product.

31. The method of claim 1, wherein the electric potential is from about 50V to about 8 kV.

32. The method of claim 1, wherein the volume ratio of the at least one first solvent to the at least one second solvent is from about 1:99 to about 99:1.

33. The method of claim 1, wherein the one or more material(s) comprise at least one polymer.

34. The method of claim 33, wherein the at least one polymer comprises at least one biocompatible polymer and/or at least one biodegradable polymer.

35. The method of claim 33, wherein the at least one polymer is thermo-reactive at a temperature of at least about 60° C.

36. The method of claim 33, wherein the at least one polymer is chosen from a polyester, polyurethane, polyether, polyketal, polyimide, polyamide, polycarbonate, polyacrylate, polysaccharide, and any combination thereof.

37. The method of claim 33, wherein the at least one polymer is chosen from polyglycolide or a polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxy valerate) (PHBV), polyethylene terephthalate (PET), polybutylene terephthalate (PBT), polyethylene glycol (PEG), polytrimethylene terephthalate (PTT), polyethylene naphthalate (PEN), poly(glycerol-sebacate) (PGS), palmitate functionalized poly(glycerol sebacate (PGSP), poly(epsilon caprolactone) (PCL), polymethyl methacrylate (PMMA), chitosan, gelatin, cellulose, polyvinyl alcohol (PVA), polyvinyl pyrrolidone (PVP), polydioxanone, derivatives thereof, and any combination thereof.

38. The method of claim 33, wherein the at least one polymer has a concentration in the fluid stock(s) of from about 5% to about 90% w/V.

39. The method of claim 33, wherein the fluid stock(s) comprising the at least one polymer is/are ejected from the nozzle(s) to form one or more fiber(s) comprising the at least one polymer.

40. The method of claim 33, wherein at least one fluid stock comprises at least one first polymer and at least one second polymer, and/or wherein at least a first fluid stock comprises at least one first polymer and at least one second fluid stock comprises at least a second polymer.

41. The method of claim 33, wherein the fluid stock(s) comprising the at least one first polymer and the at least one second polymer are ejected from the same or different nozzle(s) to form one or more fiber(s) comprising the at least one first polymer and/or the at least one second polymer.

42. The method of claim 1, wherein the fluid stock(s) further comprise(s) at least one additive.

43. The method of claim 42, wherein the at least one additive is chosen from a therapeutic agent, a dye, an indicator agent, a drug, and any combination thereof.

44. The method of claim 42, wherein the at least one additive is dissolved in or dispersed as particles in the fluid stock(s).

45. The method of claim 1, wherein the fibrous product has an inner diameter of from about 0.5 mm to about 300 mm and/or an outer diameter of from about 0.51 mm to about 300 mm.

46. The method of claim 1, wherein the average diameter of the fibers is from about 100 nm to about 500 microns.

47. The method of claim 1, further comprising one or more times during formation of the fiber(s) one or more or all of the following:
altering the volume ratio of the at least one first solvent to the at least one second solvent in the fluid stock(s);
adding at least a third solvent to the fluid stock(s);
altering the concentration of a conducting agent in the fluid stock(s); and
altering the electric potential(s) applied to the nozzle(s),
wherein fiber fusion, fiber stacking, or a combination thereof is altered.

48. A product comprising one or more layer(s) of fibers, wherein:
the fibers are arranged in a predetermined pattern;
the average diameter of the fibers is from about 100 nm to about 500 microns; and
the product comprises a desired fiber fusion and/or fiber stacking.

49. The product of claim 48, wherein each fiber comprises one or more material(s) which is/are thermo-reactive at a temperature of at least 60° C.

50. The product of claim 48, wherein the product comprises a plurality of fusion points between respective portions of at least two intersected fibers, such that for each fusion point, a bottom surface of a first fiber is bonded to a top surface of a second fiber.

51. The product of claim 50, wherein the plurality of fusion points between adjacent intersected fibers is observed at an average frequency of from about 5% to about 99%.

52. The product of claim 48, wherein: for each axial direction of the product, the adjacent fibers in different layers are aligned one over the other and are vertically stacked or vertically staggered.

53. The product of claim 48, wherein each layer comprises one or more group(s) of fibers optionally aligned in one or more axial directions of the product within and/or between the layer(s).

54. The product of claim 53, wherein the group(s) of fibers is/are uniaxially, biaxially, or multi-axially oriented within and/or between the layer(s).

55. The product of claim 53, wherein each group of fibers has a substantially constant winding angle.

56. The product of claim 55, wherein the winding angle comprises an angle between from about 1° to about 89°.

57. The product of claim 48, wherein the predetermined pattern of fibers defines in the product a plurality of pores extending at least partially through the product.

58. The product of claim 57, wherein the average width of the pores is at least 1 micron.

59. The product of claim 57, wherein the pores are characterized by a cross-sectional shape in the form of a cube, a cuboid, a rhombohedron, or a rhomboid.

60. The product of claim 48, wherein the one or more material(s) comprise(s) at least one polymer.

61. The product of claim 60, wherein the at least one polymer comprises at least one biocompatible polymer and/or at least one biodegradable polymer.

62. The product of claim 60, wherein the at least one polymer is thermo-reactive at a temperature of at least about 60° C.

63. The product of claim 60, wherein the at least one polymer is chosen from a polyester, polyurethane, polyether, polyketal, polyamide, polyimide, polycarbonate, polyacrylate, polysaccharide, and any combination thereof.

64. The product of claim 63, wherein the at least one polymer is chosen from polyglycolide or a polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxy valerate) (PHBV), polyethylene terephthalate (PET), polybutylene terephthalate (PBT), polyethylene glycol (PEG), polytrimethylene terephthalate (PTT), polyethylene naphthalate (PEN), poly(glycerol-sebacate) (PGS), palmitate functionalized poly(glycerol sebacate (PGSP), poly(epsilon caprolactone) (PCL), polymethyl methacrylate (PMMA), chitosan, gelatin, cellulose, polyvinyl alcohol (PVA), polyvinyl pyrrolidone (PVP), polydioxanone, derivatives thereof, and any combination thereof.

65. The product of claim 57, wherein at least one fiber comprises at least one first polymer and at least one second polymer, and/or wherein at least one first fiber comprises at least one first polymer and at least second fiber comprises at least one second polymer.

66. The product of claim 48, wherein at least one fiber further comprises at least one additive.

67. The product of claim 66, wherein the at least one additive is chosen from a therapeutic agent, a dye, an indicator agent, a drug, and any combination thereof.

68. The product of claim 48, wherein the product has an inner diameter of from about 0.5 mm to about 300 mm and/or an outer diameter of from about 0.51 mm to about 600 mm.

69. The product of claim 48, wherein the product is a conduit, a web, a patch, a mat, or a cuff.

70. The product of claim 48, wherein the product comprises a shape of at least a portion of an organ, a vessel, or a body part.

71. The product of claim 48, wherein the product is an implantable medical device or a scaffold of an artificial tissue.

72. The product of claim 48, wherein the product is an arteriovenous graft.

73. The product of claim 72, wherein the arteriovenous graft comprises a first orifice and a second orifice.

74. The product of claim 73, wherein the first orifice comprises an inner linear dimension that is from about 10% to about 1000% larger than an inner linear dimension of the second orifice.

75. The product of claim 72, wherein the arteriovenous graft comprises a first end and a second end.

76. The product of claim 75, wherein the first end comprises an inner diameter and the second end comprises an inner diameter, and the ratio of first end inner diameter to second end inner diameter is from about 1.5:1 to about 10:1 and/or the ratio of first end wall thickness to second end wall thickness is from about 1:1.25 to about 1:100.

77. A solution electrowriting system comprising:
a plurality of nozzles;
a material supply system comprising one or more reservoir(s) fluidically coupled to the nozzles and configured to supply one or more fluid stock(s) to the nozzles thereby ejecting one or more jet stream(s) of the fluid stock(s) from the nozzles;
a collector system configured to collect one or more fiber(s) formed by the jet stream(s) ejected from the nozzles; and
one or more power source(s) configured to provide one or more electric potential(s) to each of the nozzles and, optionally, to the collector system, thereby providing one or more electric potential difference(s) between the collector system and each of the nozzles.

78. The system of claim 77, the plurality of nozzles comprises one or more array(s) of nozzles.

79. The system of claim 78, wherein the one or more array(s) of nozzles comprise(s) a linear array of nozzles and/or a radial array of nozzles.

80. The system of claim 78, wherein the nozzles have a tip-to-tip separation distance of from about 1 mm to about 300 mm.

81. The system of claim 77, wherein the plurality of nozzles comprise a first nozzle or a first array of nozzles configured to form a group of fibers aligned in a first direction, and a second nozzle or a second array of nozzles configured to form a group of fibers aligned in a second direction, wherein the first direction and the second direction form an angle with a degree of from about 0° to about 90°.

82. The system of claim 77, further comprising a motorized stage configured to move one or more or all of the nozzles or one or more array(s) of the nozzles parallel to the longitudinal axis of the collector system during the electrowriting; and/or wherein one or more or all of the nozzles or one or more array(s) of the nozzles is/are configured to move parallel to the longitudinal axis of the collector system during electrowriting.

83. The system of claim 77, wherein the collector system is positioned at a distance from the nozzles of from about 500 microns to about 50 mm.
